(19) United States
(12) Patent Application Publication
(10) Pub. No.: US 2022/0249485 A1
(43) Pub. Date: Aug. 11, 2022

(54) TOPICAL COMPOSITIONS AND METHODS OF FORMULATING THE SAME

(71) Applicant: CMPD Licensing, LLC, Conroe, TX (US)

(72) Inventor: Jay Richard Ray, II, Conroe, TX (US)

(73) Assignee: CMPD Licensing, LLC, Conroe, TX (US)

(21) Appl. No.: 17/728,078

(22) Filed: Apr. 25, 2022

Related U.S. Application Data
(63) Continuation-in-part of application No. 16/790,395, filed on Feb. 13, 2020.
Application No. 16/790,395 is a continuation-in-part of application No. 16/381,975, filed on Apr. 11, 2019.
Application No. 16/381,975 is a continuation-in-part of application No. 16/423,937, filed on May 28, 2019.
Application No. 16/423,937 is a continuation-in-part of application No. 16/702,085, filed on Dec. 3, 2019, now Pat. No. 11,173,163.
Application No. 16/702,085 is a continuation-in-part of application No. 15/881,009, filed on Jan. 26, 2018, now Pat. No. 10,898,491.
Application No. 15/881,009 is a continuation-in-part of application No. 16/270,335, filed on Feb. 7, 2019.
Application No. 15/881,009 is a continuation-in-part of application No. 15/625,989, filed on Jun. 16, 2017, now abandoned.
Application No. 15/625,989 is a continuation-in-part of application No. 14/975,172, filed on Dec. 18, 2015, now Pat. No. 9,707,229.
Application No. 14/975,172 is a continuation-in-part of application No. 15/440,800, filed on Feb. 23, 2017, now abandoned.
Application No. 16/270,335 is a continuation-in-part of application No. 15/976,579, filed on May 10, 2018, now Pat. No. 11,278,590.
Application No. 15/976,579 is a continuation-in-part of application No. 14/990,168, filed on Jan. 7, 2016, now Pat. No. 10,898,455.
Application No. 14/990,168 is a continuation-in-part of application No. 15/597,936, filed on May 17, 2017, now Pat. No. 10,105,342.
Application No. 15/597,936 is a continuation-in-part of application No. 15/668,184, filed on Aug. 3, 2017.
Application No. 15/597,936 is a continuation-in-part of application No. 15/440,800, filed on Feb. 23, 2017, now abandoned.
Application No. 15/440,800 is a continuation-in-part of application No. 14/975,172, filed on Dec. 18, 2015, now Pat. No. 9,707,229.
Application No. 14/975,172 is a continuation-in-part of application No. 14/819,342, filed on Aug. 5, 2015, now Pat. No. 10,973,804.
(60) Provisional application No. 62/298,994, filed on Feb. 23, 2016.
(60) Provisional application No. 62/298,991, filed on Feb. 23, 2016.
(60) Provisional application No. 62/370,571, filed on Aug. 3, 2016.

(51) Int. Cl. A61K 31/513; A61K 31/7036; A61K 38/14; A61K 47/02; A61K 47/36; A61K 47/26

(52) U.S. Cl. CPC A61K 31/513; A61K 31/7036; A61K 47/26; A61K 47/02; A61K 47/36; A61K 38/14

(57) ABSTRACT

A topical composition may include flucytosine, corn starch, lactose, talc, PEG-8, PEG-75, water, Spiraea ulmaria flower extract, zinc acetate, and propylene glycol.

### CROSS-REFERENCE TO RELATED APPLICATIONS

This patent application is a continuation in-part application of co-pending U.S. patent application Ser. No. 16/790,395, filed Feb. 13, 2020, which is a continuation in-part application of application Ser. No. 16/381,975, filed Apr. 11, 2019, U.S. patent application Ser. No. 16/423,937, filed May 28, 2019, and U.S. patent application Ser. No. 16/702,085, filed Dec. 3, 2019, each of which is hereby incorporated by reference in its entirety. U.S. patent application Ser. No. 16/702,085 is a continuation in-part of U.S. patent application Ser. No. 15/881,009, filed Jan. 26, 2018 and U.S. patent application Ser. No. 16/270,335, filed Feb. 7, 2019. U.S. patent application Ser. No. 15/881,009 is a continuation in-part of U.S. patent application Ser. No. 15/625,989, filed Jun. 16, 2017, which is a continuation-in-part of U.S. patent application Ser. No. 14/975,172 (now U.S. Pat. No. 9,707,229), filed Dec. 18, 2015, and U.S. patent application Ser. No. 15/440,800, filed Feb. 23, 2017. U.S. patent application Ser. No. 16/270,335 is a continuation-in-part of U.S. patent application Ser. No. 15/976,579, filed May 10, 2018, which is a continuation-in-part of U.S. patent application Ser. No. 14/990,168, filed Jan. 7, 2016, U.S. patent application Ser. No. 15/597,936, filed May 17, 2017, and U.S. patent application Ser. No. 15/668,184, filed Aug. 3, 2017. U.S. patent application Ser. No. 15/597,936 is a continuation-in-part application of U.S. patent application Ser. No. 15/440,800, filed Feb. 23, 2017, U.S. patent application Ser. No. 14/975,172, (now U.S. Pat. No. 9,707,229) filed Dec. 18, 2015, and U.S. patent application Ser. No. 14/819,342, filed Aug. 5, 2015. U.S. patent application Ser. No. 15/440,800 claims the benefit of U.S. Provisional Patent Application No. 62/298,991, filed Feb. 23, 2016, and U.S. Provisional Patent Application No. 62/289,994, filed Feb. 23, 2016. U.S. patent application Ser. No. 15/668,184 claims the benefit of U.S. Provisional Patent Application No. 62/370,571, filed on Aug. 3, 2016. Each of the provisional and nonprovisional patent applications listed above is hereby incorporated by reference in its entirety.

### TECHNOLOGY FIELD

The present application relates to medicated topical compositions, methods of formulating medicated topical compositions, and methods of using medicated topical compositions to treat or prevent an infection, wound, skin disease or condition, or for anesthetic applications.

### BACKGROUND

The body normally serves as host for a variety of bacteria and fungi. Most of the time, the balance between the body as host and the microorganisms is maintained. However, there are times when the physiological, biochemical, and/or environmental conditions permit the microorganisms to tip that balance, thereby causing an infection.

Bacterial skin infections include erysipelas, impetigo, MRSA, cellulitis, folliculitis, carbuncles, and hidradenitis suppurativa. These infections can be painful, unsightly, and difficult to treat. Fungal skin infections include thrush, ringworm, yeast infections, nail infections, and diaper rash. Similar to bacterial infections, fungal skin infections can be painful, unsightly, and difficult to treat.

Feet and hands may be infected with bacteria or fungus. These infections present difficult issues for physicians to treat because of the biomechanical complexities of the extremity and the underlying circumstances that cause the infections. Soft tissue infections in the foot consist of any infectious process affecting the skin, subcutaneous tissue, adipose tissue, superficial or deep fascia, ligaments, tendons, tendon sheaths, joints, and/or joint capsules. Considering that there are more than 20 joints, 44 tendons, about 100 ligaments, 4 major compartments, and numerous fascial planes in the normal foot, the potential for complex problems is high.

Bacterial infections of the feet can occur as collections of pus, such as an abscess following a puncture wound or an infected hair follicle. These types of infections are usually red and elevated, and sometimes can be mistaken for an insect bite. There are many types of bacteria that cause an abscess, but staph are a leading cause. Bacterial skin infections can also resemble a rash, appearing as a reddened, tender, and warm area of skin. This type of infection is called cellulitis and can spread quickly, leading to red streaks that move from the foot toward the leg. The appearance of streaks is known as lymphangitis, which means the infection is spreading toward the lymph nodes. Cellulitis and lymphangitis can be caused by a variety of types of bacteria, but staph and sometimes streptococcus are the most common causes. Any infection, especially cellulitis and lymphangitis, requires prompt medical attention to avoid further spreading and complications. If left untreated, then some infections can spread to deeper tissues, including bone.

Certain fungal infections of the skin known as tinea infections are caused by dermatophytes, which are members of the Trichophyton, Microsporum, and Epidermophyton species. These mold-like fungi thrive in warm, moist areas, thriving on the dead tissues of hair, nails, and outer skin layers. Tinea infections include tinea pedis, known as athlete's foot; tinea corporis, known as ringworm; tinea capitis, a fungal infection of the scalp that can cause hair loss; tinea cruris, known as jock itch or tinea of the groin; tinea unguum, which is tinea of the nails; and tinea versicolor, a superficial fungal infection that produces brown, tan, or white spots on the trunk of the body. Tinea infections are contagious and can be passed through direct contact or by contact with clothing, from shower and pool surfaces, and even from pets.

Athlete's foot or tinea pedis is by far the most common form, with more than 12 million people in the United States suffering from the disease per year. It presents with redness, itching, burning, cracking, scaling, swelling, and occasionally bleeding. Athlete's foot includes toe web infections, moccasin type infections, and vesicular type infections. The condition generally includes small vesicles, fissures, scaling, maceration, hyper keratinization, and eroded areas between the toes and on the plantar surface of the foot, as well as on other skin areas. For example, the nails may show thickening, pitting, and subungual debris.

Reoccurrences of the infection are frequent. For some subjects, such as those also diagnosed with diabetes or circulatory problems, or obese subjects, tinea infections and their treatment can be quite serious. The source of the affliction often is a public safety and health concern, as the occurrence of tinea pedis is higher in public areas such as locker rooms, public showers, sports facilities, and the like.

Moreover, there are at least 3 different types of nail infections caused by fungi. The most common infection is frequently caused by Trichophyton rubrum and affects the nail bed and the area beneath the nail. Another type of infection affects only the nail surface and creates white or light colored patches. This second type of fungal infection is unusual and represents only about 10% of the reported cases. A third type of fungal infection affects the nail root and usually afflicts persons with impaired immune defense. A fourth (and unusual) type is caused by an infection of yeast fungi. Infections by yeast most often only affect nails that already are infected or damaged in some way.

The fungi are invasive to the keratin nail tissue. Apart from becoming discolored and brittle, the nail may often separate from the nail bed. In addition, pain and difficulty in wearing foot apparel is often experienced. Initially, the disease affects only one nail, typically one nail of the foot, and is thereafter spread to more nails. The palms of the hands and the soles of the feet may frequently be affected as well. When the skin is affected, red spots frequently occur and the skin may peel off. Nail fungal infections are one of the hardest forms of external infection to treat, of which infections of toe nails are the most difficult to treat.

Exterior body surfaces may also be wounded due to physical injury and/or diseases or conditions. Hyperkeratotic skin conditions, for example, are marked by a thickening of the outer layers of skin. While skin thickening causing common corns and calluses is a normal protection mechanism for skin, hyperkeratosis may result from irritations such as chemical contact, infections, and sunlight. Propensity for hyperkeratotic episodes may also be genetically linked, which may occur despite lack of abnormal irritation to the skin region. Individuals experiencing hyperkeratosis may experience discomfort, which may be accompanied by severe pain. Treatments for hyperkeratosis include surgical removal, e.g., cryosurgery, scalpel, laser. Chronic eczema and lichen planus may be treated with a corticosteroid. Diabetes is a disease that may be accompanied by slow healing wounds. Diseases or conditions related to the immune system may also result in wounds and/or slow healing wounds. Medical treatments and surgeries may also be accompanied by wounded tissues. Pain may accompany infections and wounds as well as nerve or muscle conditions.

### SUMMARY

In one aspect, a topical composition may include flucytosine, corn starch, lactose, talc, PEG-8, PEG-75, water, Spiraea ulmaria flower extract, zinc acetate, and propylene glycol.

In one example, the topical composition includes between about 250 mg and about 1500 mg, such as between about 250 mg and about 1000 mg, or between about 250 mg and about 750 mg, of the flucytosine.

In another aspect, a method of treating an infection in a human subject includes administering a topical composition to fungal infected tissue wherein the topical composition comprises flucytosine, corn starch, lactose, and talc.

In one example, the flucytosine is present in an amount between about 250 mg and about 1500 mg, such as between about 250 mg and about 1000 mg, or between about 250 mg and about 750 mg.

In one example, the topical composition is administered via nebulizing, spraying, or irrigating the infected tissue.

In one example, the infected tissue is selected from skin, lungs, or mucosa of the upper respiratory tract.

In one example, the topical composition is administered in a format selected from a powder, solution, or ointment.

In a further example, the flucytosine is present in an amount between about 250 mg and about 1500 mg, such as between about 250 mg and about 1000 mg, or between about 250 mg and about 750 mg. The topical composition may be administered in an ointment format and further comprises PEG-8 and PEG-75, water, Spiraea ulmaria flower extract, zinc acetate, and propylene glycol. In one example, the infected tissue is skin.

In an example, the topical composition is administered in a solution format comprising an aqueous solution or suspension. The infected tissue may be selected from lungs or mucosa of the upper respiratory tract. The infected tissue may be the lungs, and the topical composition may be administered via nebulization. In a further example, the infected tissue is mucosa of the upper respiratory tract, and the topical composition is administered via spray, drops, or irrigation.

In one example, the topical composition is administered in a powder format. The infected tissue may be skin or nasal mucosa. In an example, the infected tissue is mucosa of the upper respiratory tract, and the topical composition is administered nasally by inhalation or irrigation.

In one example, the topical composition is administered in a solution format via small particle nebulization.

In one example, the topical composition comprises one or more pharmaceutical drugs selected from an additional antifungal, antibacterials, antivirals, NSAIDs, antidepressants, local anesthetics, statins, keratolytics, acid reducers, steroids, calcium channel blockers, antianxiety drugs, mucolytics, antihistamines, or combinations thereof in an amount between about 0.001% and about 50% by weight of the topical composition.

In an above or another example, the one or more pharmaceutical drugs include one or more antibacterial drugs selected from amoxicillin, ampicillin, azithromycin, cefaclor, cefadroxil, cefazolin, cefepime, cefixime, cefpodoxime, cefprozil, ceftriaxone, cefuroxime, ceftazidime, ciprofloxacin, clarithromycin, clindamycin, colistimethate, doxycycline, erythromycin, gentamicin, isoniazid, levofloxacin, linezolid, ofloxacin, nafcillin, nitrofurantoin, mupirocin, tetracycline, tobramycin, vancomycin, and combinations thereof.

In an above or another example, the one or more pharmaceutical drugs include one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, and combinations thereof.

In an above or another example, the one or more pharmaceutical drugs include one or more antidepressants selected from amitriptyline, amoxapine, desipramine, doxepin, imipramine, nortriptyline, protriptyline, trimipramine, duloxetine, milnacipran, venlafaxine, levomilnacipran, bupropion, mirtazapine, nefazodone, trazodone, vilazodone, vortioxetine, and combinations thereof.

In an above or another example, the one or more pharmaceutical drugs include one or more NSAIDs selected from meloxicam, piroxicam, aspirin, diflunisal, salsalate, trilisate, flurbiprofen, ibuprofen, ketoprofen, naproxen, oxaprozin, diclofenac, etodolac, indomethacin, ketorolac, nabumetone, sulindac, tolmetin, celecoxib, rofecoxib, valdecoxib, celecoxib, etodolac, indomethacin, nabumetone, and combinations thereof.

In an above or another example, the one or more pharmaceutical drugs include one or more statins selected from atorvastatin, fluvastatin, pravastatin, rosuvastatin, simvastatin, and combinations thereof.

In an above or another example, the one or more pharmaceutical drugs include a steroid selected from triamcinolone, betamethasone, dexamethasone, flunisolide, prednisone, prednisolone, methylprednisolone, fluocinolone, budesonide, diflorasone, halcinonide, desoximetasone, diflucortolone, flucloronide, fluocinonide, fluocortolone, fluprednidene, flurandrenolide, clobetasol, clobetasone, alclometasone, flumethasone, fluocortolone, amcinonide, beclomethasone, fluticasone, difluprednate, prednicarbate, flurandrenolide, mometasone, desonide, halobetasol propionate, triamcinolone acetonide, and combinations thereof.

In an above or another example, the one or more pharmaceutical drugs include a local anesthetic comprising one or more of lidocaine, prilocaine, or benzocaine; an anticonvulsant comprising one or more of gabapentin, topiramate, carbamazepine, or lamotrigine; an antianxiety drug comprising buspirone; an antihistamine comprising one or more of acrivastine, azelastine, bilastine, bromodiphenhydramine, brompheniramine, buclizine, carbinoxamine, cetirizine, chlorodiphenhydramine, chlorphenamine, chlorpheniramine, chlorpromazine, cimetidine, clemastine, cyclizine, cyproheptadine, desloratadine, dexbrompheniramine, dexchlorpheniramine, dimenhydrinate, dimetindene, diphenhydramine, doxylamine, ebastine, embramine, emedastine, famotidine, fexofenadine, hydroxyzine, lafutidine, levocabastine, loratadine, meclozine, mirtazapine, nizatidine, olopatadine, orphenadrine, phenindamine, pheniramine, phenyltoloxamine, promethazine, pyrilamine, quetiapine, ranitidine, roxatidine, rupatadine, tiotidine, tripelennamine, or triprolidine; an antiviral comprising one or more of acyclovir, famciclovir, valacyclovir, or penciclovir; a calcium channel blocker comprising amlodipine; an acid reducer comprising one or both of allopurinol or sucralfate; a mucolytic comprising one or more of acetylcysteine, bromheksin, carbocisteine, erdosteine, guaifenesin, iodinated glycerol; and/or a keratolytic comprising one or more of urea, salicylic acid, or papain.

In an above or another example, the one or more pharmaceutical drugs include one or more stimulants, disinfectants, nerve depressants, muscle relaxants, NMDA (N-Methyl-D-aspartate) receptor antagonists, opiate or opioid agonists, anticholinergics, or combinations thereof in an amount between about 0.01% and about 25% by weight of the topical composition.

### DESCRIPTION

A composition according to the present disclosure may include a composition formulated to be topically applied to an external surface of a mammal, such as a human. In some embodiments, the composition may be formulated to be administered to skin. Embodiments of the composition may also be formulated to be administered to the skin, vagina, nasal, or anal cavity. For example, the composition may be administered to skin of the head, neck, torso, or lower body. In some embodiments, the composition may be administered to nails. Such compositions may be referred to herein as a topical composition.

In some embodiments, the topical composition may include one or more active agents comprising one or more pharmaceuticals drugs, such as antifungals, antibacterials, antivirals, nonsteroidal anti-inflammatory drugs (NSAIDs), local anesthetics, keratolytics, statins, antidepressants, anticonvulsants, steroids, anesthetics, acid reducers, calcium channel blockers, antianxiety drugs, mucolytics, antihistamines, or combinations thereof. Additionally or alternatively, the topical composition may comprise one or more active agents selected from NMDA (N-Methyl-D-aspartate) receptor antagonists, muscle relaxants, anticholinergics, stimulants, or combinations thereof. The topical composition may include a carrier comprising one or more carriers, which may be used interchangeably with the term base. The carrier may be liquid, semi-liquid, or solid. For example, the carrier may include an aqueous, organic, or inorganic solution, which may include a dispersion or suspension, cream, gel, ointment, lotion, emulsion, powder, or paste.

The topical composition may be formulated to treat microbial infections, such as infections of the skin, nails, mucosal surfaces, and potentially internalized infections, e.g., via transdermal administration of antimicrobial agents. The topical composition may be formulated to treat wounds or damaged tissue, including broken and/or unbroken skin. The topical composition may be formulated to treat neurological pain and/or muscle pain. In some embodiments, the topical composition may treat pain accompanying diseases and conditions, such as diabetic neuropathy, radiculopathy, or shingles. The topical composition may be formulated to treat inflammation caused by arthritis or gout, for example. The topical composition may be formulated to assist in debridement of wounds.

In some embodiments, the topical composition comprises one or more antimicrobial drugs (antiviral, antifungal, and/or antibacterial drugs) alone or in combination with one or more additional actives selected from one or more anti-inflammatory drugs (e.g., NSAIDs, steroids, antidepressants), an anti-allergy drugs (e.g., antihistamines), antidepressants, stimulant drugs, disinfectants, anticonvulsants, local anesthetics, or combinations thereof. In one embodiment, the topical composition includes additional active agents selected from one or more anticonvulsants, nerve depressants, muscle relaxants, NMDA (N-Methyl-D-aspartate) receptor antagonists, opiate or opioid agonists, antidepressants, anticholinergics and/or other active agents. In some embodiments, the topical composition may comprise the antimicrobial agent including an antifungal agent, antibacterial agent, or both alone or in combination with a steroid, antiviral, NSAID, antidepressant, anticonvulsant, analgesic, opiate or opioid agonist, keratolytic, or combination thereof. The active agents. including associated drugs, identified herein may include pharmaceutically acceptable salts and derivatives of the identified active agents.

In various embodiments, the topical composition comprises one or more antifungal drugs comprising selected from one or more categories of antifungals including azoles (imidazoles), antimetabolites, allylamines, morpholine, glucan synthesis inhibitors (echinocandins), polyenes, benoxaaborale; other antifungal/onychomycosis agents, and new classes of antifungal/onychomycosis agents. For example, the antifungal may comprise one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. In some embodiments, the antifungal is selected from one or more azoles. In one embodiment, the antifungal is selected from itraconazole, voriconazole, or combination thereof. In various embodiments, the topical composition may comprise between about 0.01% and about 50% by weight antifungal, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%, between about 20% and about 50%, between about 20% and about 40%, between about 20% and about 30%, between about 30% and about 50%, between about 30% and about 50%, or between about 40% and about 50%. In some embodiments, the amount of antifungal by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, about 25%, about 30%, about 35%, about 40%, about 45%, about 50%, or any other percentage between about 0.01% and 50% by weight of the topical composition.

In various embodiments, the topical composition includes one or more of the above antifungals in combination with one or more antibacterials, antivirals, NSAIDs, keratolytics, statins, antidepressants, anticonvulsants, steroids, anesthetics, acid reducers, calcium channel blockers, antianxiety drugs, mucolytics, or antihistamines, including combinations thereof. In an above or another embodiment, the topical composition comprises the one or more antifungals in combination with one or more stimulants, disinfectants, nerve depressants, muscle relaxants, NMDA (N-Methyl-D-aspartate) receptor antagonists, opiate or opioid agonists, anticholinergics and/or other active agents. In some embodiments, the topical composition may comprise the one or more antifungals in combination with one or more antibacterials, steroids, antivirals, NSAIDs, antidepressants, anticonvulsants, analgesics, opioids, keratolytics, or combinations thereof.

In various embodiments, the topical composition comprises one or more antibacterial drugs selected from enicillins, cephalosporins, fluoroquinolones, aminoglycosides, monobactams, carbapenems, macrolides, other antibacterials, or combination thereof. For example, the topical composition may include one or more antibacterials selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In some embodiments, the antibacterial agent is selected from mupirocin, gentamycin, tobramycin, or combinations thereof. In one embodiment, the antibacterial agent includes an aminoglycoside.

In various embodiments, the topical composition includes one or more antibacterials selected vancomycin, ciprofloxacin, levofloxacin, azithromycin, clindamycin, doxycycline, mupirocin, ceftriaxone, colistimethate, tobramycin, cefepime, gentamicin, streptomycin, sulfamethoxazole/trimethoprim, or combinations thereof. In one example, the topical composition comprises linezolid, levofloxacin, ciprofloxacin, or combination thereof. In some embodiments, the topical composition comprises one or more antibacterial drugs selected from amoxicillin, ampicillin, azithromycin, cefaclor, cefadroxil, cefazolin, cefepime, cefixime, cefpodoxime, cefprozil, ceftriaxone, cefuroxime, ceftazidime, ciprofloxacin, clarithromycin, clindamycin, colistimethate, doxycycline, erythromycin, gentamicin, isoniazid, levofloxacin, linezolid, ofloxacin, nafcillin, nitrofurantoin, mupirocin, tetracycline, tobramycin, vancomycin, and combinations thereof.

In various embodiments, the topical composition comprises one or more above antibacterials in combination with one or more antifungals, antivirals, NSAIDs, keratolytics, statin, antidepressants, anticonvulsants, steroids, anesthetics, acid reducers, calcium channel blockers, antianxiety drugs, mucolytics, or antihistamines, including combinations thereof. In an above or another embodiment, the topical composition comprises the one or more antibacterials in combination with one or more stimulants, disinfectants, nerve depressants, muscle relaxants, NMDA (N-Methyl-D-aspartate) receptor antagonists, opiate or opioid agonists, anticholinergics and/or other active agents. In some embodiments, the topical composition comprises the one or more antibacterials in combination with one or more antifungals, steroids, antivirals, NSAIDs, antidepressants, anticonvulsants, analgesics, opioids, keratolytics, or combinations thereof.

In various embodiments, the topical composition may comprise between about 0.01% and about 50% by weight antibacterial, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%, between about 20% and about 50%, between about 20% and about 40%, between about 20% and about 30%, between about 30% and about 50%, between about 30% and about 50%, or between about 40% and about 50%. In some embodiments, the amount of antibacterial by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, about 25%, about 30%, about 35%, about 40%, about 45%, about 50%, or any other percentage between about 0.01% and 50% by weight of the topical composition.

In various embodiments, the topical composition includes one or more antivirals selected from acyclovir, famciclovir, valacyclovir, penciclovir, or combinations thereof. In various embodiments, the topical composition comprises one or more above antivirals in combination with one or more antifungals, antibacterials, antidepressants, NSAIDs, keratolytics, anticonvulsants, local anesthetics, steroids, statins, acid reducers, calcium channel blockers, antianxiety drugs, mucolytics, or antihistamines, including combinations thereof. In an above or another embodiment, the topical composition comprises the one or more antivirals in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition may comprise between about 0.01% and about 50% by weight antiviral, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%, between about 20% and about 50%, between about 20% and about 40%, between about 20% and about 30%, between about 30% and about 50%, between about 30% and about 50%, or between about 40% and about 50%. In some embodiments, the amount of antiviral by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, about 25%, about 30%, about 35%, about 40%, about 45%, about 50%, or any other percentage between about 0.01% and 50% by weight of the topical composition.

In various embodiments, the topical composition includes one or more antidepressants selected from one or more tricyclic antidepressants: amitriptyline, amoxapine, desipramine, doxepin, imipramine, nortriptyline, protriptyline, and/or trimipramine. In one embodiment, the antidepressant comprises one or more antidepressants selected from amitriptyline, doxepin, duloxetine, milnacipran, nortriptyline, venlafaxine, levomilnacipran, desipramine, or combinations thereof. In one embodiment, an antidepressant may include bupropion, mirtazapine, nefazodone, trazodone, vilazodone, and/or vortioxetine. In various embodiments, the topical composition comprises one or more above antidepressants in combination with one or more antifungals, antibacterials, antivirals, NSAIDs, keratolytics, anticonvulsants, local anesthetics, steroids, statins, acid reducers, calcium channel blockers, antianxiety drugs, mucolytics, or antihistamines, including combinations thereof. In an above or another embodiment, the topical composition comprises the one or more antidepressants in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition may comprise between about 0.01% and about 20% by weight antidepressant, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%. In some embodiments, the amount of antidepressant by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, or any other percentage between about 0.01% and 20% by weight of the topical composition.

In various embodiments, the topical composition includes one or more nonsteroidal anti-inflammatory drug (NSAID)selected from oxicams, such as meloxicam or piroxicam; salicylic acid derivatives, such as aspirin, diflunisal, salsalate, or trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, or tolmetin; fenamates, such as meclofenamate; and/or COX-2 inhibitors, such as celecoxib, rofecoxib, or valdecoxib. In one embodiment, the one or more NSAIDs are selected from celecoxib, etodolac, indomethacin, nabumetone, and combinations thereof.

In various embodiments, the topical composition comprises one or more above NSAIDs in combination with one or more antifungals, antibacterials, antivirals, keratolytics, statins, antidepressants, anticonvulsants, steroids, anesthetics, acid reducers, calcium channel blockers, antianxiety drugs, mucolytics, or antihistamines, including combinations thereof. In an above or another embodiment, the topical composition comprises the one or more NSAIDs in combination with one or more stimulants, disinfectants, nerve depressants, muscle relaxants, NMDA (N-Methyl-D-aspartate) receptor antagonists, opiate or opioid agonists, anticholinergics and/or other active agents. In some embodiments, the topical composition comprises the one or more NSAIDs in combination with one or more antibacterials, antifungals, steroids, antivirals, antidepressants, anticonvulsants, analgesics, opioids, keratolytics, or combinations thereof.

In various embodiments, the topical composition may comprise between about 0.01% and about 50% by weight NSAID, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%, between about 20% and about 50%, between about 20% and about 40%, between about 20% and about 30%, between about 30% and about 50%, between about 30% and about 50%, or between about 40% and about 50%. In some embodiments, the amount of NSAID by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, about 25%, about 30%, about 35%, about 40%, about 45%, about 50%, or any other percentage between about 0.01% and 50% by weight of the topical composition.

In various embodiments, the topical composition includes one or more statins selected from atorvastatin, fluvastatin, pravastatin, rosuvastatin, simvastatin, or combinations thereof.

In various embodiments, the topical composition comprises one or more above statins in combination with one or more antifungals, antibacterials, antivirals, NSAIDs, keratolytics, antidepressants, anticonvulsants, local anesthetics, steroids, acid reducers, calcium channel blockers, antianxiety drugs, mucolytics, or antihistamines, including combinations thereof. In an above or another embodiment, the topical composition comprises the one or more statins in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition may comprise between about 0.01% and about 20% by weight statin, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%. In some embodiments, the amount of statin by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, or any other percentage between about 0.01% and 20% by weight of the topical composition.

In various embodiments, the topical composition includes one or more local anesthetics selected from lidocaine, prilocaine, benzocaine, or combination thereof.

In various embodiments, the topical composition comprises one or more above local anesthetics in combination with one or more antifungals, antibacterials, antivirals, NSAIDs, keratolytics, statins, antidepressants, anticonvulsants, steroids, acid reducers, calcium channel blockers, antianxiety drugs, mucolytics, or antihistamines, including combinations thereof. In an above or another embodiment, the topical composition comprises the one or more local anesthetics in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition may comprise between about 0.01% and about 15% by weight local anesthetic, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%. In some embodiments, the amount of local anesthetic by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, or any other percentage between about 0.01% and 15% by weight of the topical composition.

In various embodiments, the topical composition includes one or more steroids selected from triamcinolone (e.g., diacetate, hexacetonide, and acetonide), betamethasone (e.g., dipropionate, benzoate, sodium phosphate, acetate, and valerate), dexamethasone (e.g., dipropionate and valerate), flunisolide, prednisone (e.g., acetate), prednisolone (e.g., acetate, sodium phosphate, and tebutate), methylprednisolone (e.g., acetate and sodium succinate), fluocinolone (e.g., acetonide), budesonide, diflorasone (e.g., diacetate), halcinonide, desoximetasone (desoxymethasone), diflucortolone (e.g., valerate), flucloronide (fluocortolone acetonide), fluocinonide, fluocortolone, fluprednidene (e.g., acetate), flurandrenolide (flurandrenolone), clobetasol (e.g., propionate), clobetasone (e.g., butyrate), alclometasone, flumethasone (e.g., pivalate), fluocortolone (e.g., hexanoate), amcinonide, beclomethasone (e.g., dipropionate), fluticasone (e.g., propionate), difluprednate, prednicarbate, flurandrenolide, mometasone, desonide, halobetasol propionate, triamcinolone acetonide, or combination thereof. In one embodiment, the topical composition comprises fluticasone.

In various embodiments, the topical composition includes one or more of the above steroids in an amount about 0.25 mg and about 40 mg, such as about 0.5 mg or greater, about 1 mg or greater, about 2 mg or greater, about 3 mg or greater, about 4 mg or greater, about 5 mg or greater, about 6 mg or greater, about 8 mg or greater, about 10 mg or greater, about 12 mg or greater, about 15 mg or greater, about 20 mg or greater, about 25 mg or greater, about 30 mg or greater, about 35 mg or greater, about 0.25 mg and about 5 mg, between about 5 mg and about 15 mg, between about 0.5 mg and about 3 mg, between about 0.5 mg and about 2 mg, between about 2 mg and about 4 mg, between about 4 mg and about 4 mg and about 10 mg, between about 0.5 mg and about 10 mg, between about 10 mg and about 15 mg, between about 15 mg and about 20 mg, between about 20 mg and about 25 mg, between about 30 mg and about 35 mg, between about 35 mg and about 40 mg in a dosage volume.

In various embodiments, the topical composition may comprise between about 0.001% and about 50% by weight steroid, such as between about 0.001% and about 1%, between about 1% and about 3%, between about 3% and about 5%, between about 5% and about 7%, between about 7% and about 10%, between about 10% and about 15%, between about 15% and 30%, between about 30% and about 40%, between about 40% and about 50%, greater than about 5%, greater than about 7%, greater than about 10%, greater than about 15%, greater than about 20%, greater than about 30%, or greater than about 40% steroid by weight. In some embodiments, the amount of steroid by weight may be about 0.001%, about 0.005%, about 0.01%, about 0.05%, about 0.1%, about 0.5%, about 0.8%, about 1%, about 1.2%, about 1.6%, about 1.8%, about 2%, about 2.5%, about 3%, about 4%, about 5%, about 7%, about 10%, about 15%, about 20%, about 25%, about 30%, about 35%, about 40%, about 45% or any other percentage between about 0.001% and about 50% by weight of the topical composition.

In various embodiments, the topical composition comprises one or more above steroids in combination with one or more antifungals, antibacterials, antivirals, NSAIDs, keratolytics, statins, antidepressants, anticonvulsants, local anesthetics, acid reducers, calcium channel blockers, antianxiety drugs, mucolytics, or antihistamines, including combinations thereof. In an above or another embodiment, the topical composition comprises the one or more steroids in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition includes one or more anticonvulsants selected from gabapentin, topiramate, carbamazepine, lamotrigine, or combinations thereof.

In various embodiments, the topical composition comprises one or more above anticonvulsants in combination with one or more antifungals, antibacterials, antivirals, NSAIDs, keratolytics, statins, antidepressants, local anesthetics, steroids, acid reducers, calcium channel blockers, antianxiety drugs, mucolytics, or antihistamines, including combinations thereof. In an above or another embodiment, the topical composition comprises the one or more anticonvulsants in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition may comprise between about 0.01% and about 20% by weight anticonvulsants, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%. In some embodiments, the amount of anticonvulsant by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, or any other percentage between about 0.01% and 20% by weight of the topical composition.

In various embodiments, the topical composition comprises one or more antianxiety drugs (e.g., buspirone), calcium channel blockers (e.g., amlodipine), and/or antihistamines. Antihistamines may be selected from acrivastine, azelastine, bilastine, bromodiphenhydramine, brompheniramine, buclizine, carbinoxamine, cetirizine, chlorodiphenhydramine, chlorphenamine, chlorpheniramine, chlorpromazine, cimetidine, clemastine, cyclizine, cyproheptadine, desloratadine, dexbrompheniramine, dexchlorpheniramine, dimenhydrinate, dimetindene, diphenhydramine, doxylamine, ebastine, embramine, emedastine, famotidine, fexofenadine, hydroxyzine, lafutidine, levocabastine, loratadine, meclozine, mirtazapine, nizatidine, olopatadine, orphenadrine, phenindamine, pheniramine, phenyltoloxamine, promethazine, pyrilamine, quetiapine, ranitidine, roxatidine, rupatadine, tiotidine, tripelennamine, triprolidine, or combination thereof. The antianxiety drugs, calcium channel blockers, and/or antihistamines may be present in an amount, together or individually, between about 0.001% and about 15% by weight. For example, the topical composition may comprise between about 0.001% and about 0.05%, between about 0.01% and about 3%, between about 0.01% and about 1%, between about 0.01% and about 0.25%, between about 0.01% and about 0.15%, between about 0.05% and about 0.15%, between about 0.1% and about 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 14% and about 15% by weight. In one embodiment, antihistamines may be present in an amount between about 0.0001% and about 5% by weight, such as between about 0.0005% and about 0.5%. In some embodiments, the amount of antianxiety drug, calcium channel blocker, and/or antihistamine by weight, together or individually, may be about 0.002%, about 0.005%, about 0.008%, about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, or any other percentage between about 0.001% and 15% by weight of the topical composition. The topical composition may comprise the one or more antianxiety drug, calcium channel blocker, and/or antihistamines alone or in combination with one or more antifungals, antibacterials, antivirals, NSAIDs, keratolytics, antidepressants, mucolytics, anticonvulsants, local anesthetics, steroids, statins, acid reducers, or combinations thereof.

In various embodiments, the topical composition includes one or more acid reducers. In some embodiments, the acid reducer may be selected from allopurinol, sucralfate, or both. In various embodiments, the topical composition comprises one or more above acid reducers in combination with one or more antifungals, antibacterials, antivirals, NSAIDs, keratolytics, antidepressants, anticonvulsants, local anesthetics, steroids, statins, calcium channel blockers, antianxiety drugs, mucolytics, or antihistamines, including combinations thereof. In an above or another embodiment, the topical composition comprises the one or more acid reducers in combination with one or more stimulants, disinfectants, nerve depressants, muscle relaxants, NMDA (N-Methyl-D-aspartate) receptor antagonists, opiate or opioid agonists, anticholinergics and/or other active agents.

In various embodiments, the topical composition may comprise between about 0.01% and about 20% by weight acid reducer, such as between about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20%. In some embodiments, the amount of acid reducer by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, or any other percentage between about 0.01% and 20% by weight of the topical composition.

In various embodiments, the topical composition comprises one or more mucolytics selected from acetylcysteine, bromheksin, carbocisteine, erdosteine, guaifenesin, iodinated glycerol, pharmaceutically acceptable salts thereof, or a combination thereof, in an amount between about 0.01% and about 10% by weight. For example, the topical composition may comprise about 0.01% and about 5%, between about 0.01% and about 3%, between about 0.01% and about 1%, between about 0.01% and about 0.25%, between about 0.01% and about 0.15%, between about 0.05% and about 0.15%, between 0.1% and 10%, between about 0.1% and about 0.5%, between about 0.1% and about 0.2%, between about 0.2% and about 0.8%, between about 0.2% and about 0.6%, between about 0.2% and about 0.4%, between about 0.3% and about 1%, between about 0.3% and about 0.8%, between about 0.3% and about 0.6%, between about 0.4% and about 1%, between about 0.5% and about 1%, between about 0.5% and about 8%, between about 0.6% and about 1%, between about 0.6% and about 0.8%, between about 0.8% and about 1%, between about 1% and about 3%, between about 1% and about 10%, between about 1% and about 8%, between about 1% and about 5%, between about 1% and about 3%, between about 3% and about 10%, between about 3% and about 8%, between about 3% and about 5%, between about 5% and about 10%, between about 5% and about 8%, between about 6% and about 10%, between about 6% and about 8%, between about 7% and about 10%, between about 8% and about 10%, between about 10% and about 20%, between about 10% and about 15%, between about 10% and about 12%, between about 12% and about 15%, or between about 15% and about 20% mucolytic by weight. In some embodiments, the amount of mucolytic by weight may be about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 17%, about 19%, about 20%, or any other percentage between about 0.01% and 20% by weight of the topical composition. The topical composition may comprise the one or more mucolytics alone or in combination with one or more antifungals, antibacterials, antivirals, NSAIDs, keratolytics, antidepressants, anticonvulsants, local anesthetics, steroids, statins, calcium channel blockers, antianxiety drugs, acid reducers, antihistamines, or combinations thereof.

In various embodiments, the topical composition comprises a keratolytic agent selected from urea, salicylic acid, papain, or combinations thereof. For example, the topical composition may comprise urea and one or more antifungals, antibacterials, antivirals, NSAIDs, antidepressants, anticonvulsants, local anesthetics, steroids, statins, calcium channel blockers, antianxiety drugs, acid reducers, antihistamines, or combinations thereof. In various embodiments, the topical composition may comprise between about 1% and about 50% by weight urea, such as between about 1% and about 30%, between about 5% and about 40%, between about 5% and about 30%, between about 10% and about 50%, between about 10% and about 40%, between 10% and about 30%, between about 20% and about 50%, between about 20% and about 40%, between about 20% and about 30%, between about 30% and about 50%, between about 30% and about 40%, between about 40% and about 50% by weight. In some embodiments, the amount of urea by weight may be about 5%, about 10%, about 15%, about 20%, about 25%, about 30%, about 35%, about 40%, about 45%, about 50%, or any other percentage between about 1% and 50% by weight of the topical composition.

In various embodiments, the topical composition comprises a NMDA receptor antagonist agent such as ketamine. In some embodiments, the topical composition may comprise an opiate or opioid agonist agent selected from tramadol; one or more C2 opiate agonists selected from oxycodone, morphine, methadone, hydromorphone, and fentanyl; one or more C3 opiate agonists selected from hydrocodone, codeine, propoxyphene, butalbital, and pentazocine; or any combination thereof.

In various embodiments, the topical composition comprises a muscle relaxant agent comprising one or more muscle relaxants selected from baclofen, carisoprodol, chlorzoxazone, cyclobenzaprine, dantrolene, diazepam, metaxalone, methocarbamol, orphenadrine, quinine sulfate, tizanidine, and/or other muscle relaxants. In various embodiments, the topical composition comprises between about 0.001% and about 5% by weight muscle relaxant agent.

In various embodiments, the topical composition comprises an anticholinergic agent comprising one or more anticholinergics selected from atropine, belladonna alkaloids, benzatropine, benztropine mesylate, biperiden, bupropion, chlorpheniramine, clemastine, darifenacin, dextromethorphan, dicyclomine, dimenhydrinate, diphenhydramine, doxacurium, doxepin, doxylamine, fesoterodine, flavoxate, glycopyrrolate, hexamethonium, hydroxyzine, hyoscyamine, ipratropium (e.g., ipratropium bromide), mecamylamine, orphenadrine, oxitropium, oxybutynin, procyclidine, propantheline, scopolamine, solifenacin, tiotropium, tolterodine, trihexyphenidyl, tropicamide, tubocurarine, or a combination thereof. In various embodiments, the topical composition comprises between about 0.001% and about 5% by weight anticholinergic agent.

In one example, the topical composition comprises a stimulant agent comprising one or more stimulants selected from amphetamine, armodafinil, atomoxetine, benzphetamine, caffeine, doxapram, dextroamphetamine, dexmethylphenidate, dextroamphetamine, diethylpropion, methylphenidate, methamphetamine, modafinil, pitolisant, phentermine, phendimetrazine, or combination thereof. The stimulant may comprise between about 0.001% and about 5% by weight of the topical composition. The stimulant may comprise commercially available tablets, capsules, vials for injection, bulk power, solutions, topical ointments, creams, lotions, and/or gels, for example.

The topical composition may be provided in a topical format, which may include a carrier for topical administration. In various embodiments, the topical composition may include a colloid or emulsion (o/w, w/o), cream, lotion, ointment, foam, aqueous or non-aqueous gel, aqueous or non-aqueous solution, which may include a dispersion, powder, nail lacquer, bath, or paste.

The topical composition may include a carrier comprising one or more carriers or carrier components thereof. The carrier may be liquid, semi-liquid, or solid. For example, the carrier may include an aqueous, organic, or inorganic solution, which may include a dispersion or suspension, cream, gel, ointment, lotion, emulsion, powder, or paste. In some embodiments, the carrier includes a carrier or vehicle composition such as a base cream, ointment, gel, lotion, foam, or solution. The carrier may include carrier components such as lecithin, phospholipids, glycols, paraffin, fatty acids, carbopols/carbomers, alcohols, lanolin, for example.

In some embodiments, the carrier comprises an aqueous solution. In some examples, the carriers comprising aqueous solutions may be combined with the one or more active agents to formulate a topical composition comprising an irrigation solution, a footbath, a nail lacquer, a topical spray or soak, for example. In an embodiment, the carrier or component may include an aqueous solution comprising a saline solution. For example, the topical composition may comprise a carrier or component comprising a sodium hydroxide solution, which may be a sterile solution, an alcohol, water, e.g., purified water, water for irrigation, water for injection, or a sterile water. In one embodiment, a carrier or component comprises a sodium chloride 0.09% solution (sterile). The carrier or component may be present in an amount sufficient to obtain the desired amount of active agents per unit weight or volume.

The topical composition may include a carrier comprising a polyethylene glycol (PEG)carrier component. In other embodiments, the composition is PEG-free. In these or other embodiments, the composition may include a silicon or silicon variant carrier component. In some embodiments, the composition is silicon-free. An example topical composition may comprise a solution including carrier components selected from water, alcohol, DMSO, saline or sodium chloride, sodium hypochlorite, or other aqueous or non-aqueous carrier medium into which the one or more active agents are mixed, dispersed, solubilized, or dissolved. The topical composition may be water soluble/miscible or formulated for water absorption. The topical composition may comprise a water-in-oil emulsion or oil-in-water emulsion. In one embodiment, the topical composition comprises a emulsion, e.g., a cream or lotion format, comprising one or more carrier components selected from of acrylate copolymer, alcohol, camphor, carbomer, dimethyl isosorbide, disodium EDTA, dl-alphatocopheryl acetate, edetate disodium, emulsifying wax, Eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate (e.g., polysorbate 85, polysorbate 20), purified water, titanium dioxide, tridecyl stearate, tridecyl trimellitate, sodium hydroxide, sodium hydroxide, sorbitol, stearic acid, zinc pyrithione, or combinations thereof. In some embodiments, the topical composition comprises a foam format that includes propellant carrier component such as butane. Topical compositions comprising a foam format may also comprise additional characteristics such as that of an emulsion, such as an oil-in-water emulsion, or gel.

In one example, the topical composition comprises an ointment format and includes active agents in a carrier comprising carrier components selected from hydrophilic petrolatum, white petrolatum, hydrophilic ointment, white ointment, anhydrous lanolin, hydrous lanolin, PEG ointment, or combinations thereof. In an embodiment, the topical composition comprises a gel format. The gel may be an aqueous or non-aqueous gel. The gel may include carrier components thickening agents and/or gelling agents such as carbopol, poloxamer, xanthan gum, methylcellulose, carboxymethyl cellulose, hydroxyethyl cellulose, ethylcellulose, gelatin, magnesium aluminum silicate, polyvinyl alcohol, sodium alginate, or combinations thereof. The topical composition may include a powder format and include carrier components such as lactose or talc, for example.

The topical composition or carrier thereof may include carrier components such as one or more solubilizers, stabilizers, buffers, tonicity modifiers, bulking agents, viscosity enhancers/reducers, surfactants, chelating agents, adjuvants, or combinations thereof.

In various embodiments, the topical composition or carrier thereof comprises one or more glucose polymers such as a starch, cellulose, polydextrose, or combination thereof. Example starches may include sodium starch glycolate, corn starch, pregelatinized starch, or combination thereof. Example celluloses may include hydroxypropyl cellulose, hypermellose, croscarmellose sodium, ethyl cellulose, microcrystalline cellulose, or combination thereof. Povidone such as povidone K30, copovidone, crospovidone, or combination thereof, may also be present. In some embodiments, glycol and/or a sugar alcohol may be present. Example glycols may include polyethylene glycol, propylene glycol, or combination thereof. Example sugar alcohols may include mannitol. Some embodiments may include oxides such as silicon dioxide, titanium dioxide, ferric oxide, or combination thereof. One embodiment may include any of the above and magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof. In one embodiment, the topical composition does not include one or more of starch, cellulose, polydextrose, sodium starch glycolate, corn starch, pregelatinized starch, hydroxypropyl cellulose, hypermellose, croscarmellose sodium, ethyl cellulose, microcrystalline cellulose, povidone, povidone K30, copovidone, crospovidone, polyethylene glycol, propylene glycol, mannitol, silicon dioxide, titanium dioxide, ferric oxide, magnesium stearate, talc, diethyl phthalate, sodium stearyl fumarate, sodium lauryl sulfate, polysorbate, triacetin, polacrilin, lactose, glycerol behenate, polyvinyl alcohol, carnauba wax, or combination thereof.

In some embodiments, the carrier comprises a water washable, moisturizing ointment, which may also be referred to as Bassa-Gel herein, comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel.

The topical composition may be administered topically by contacting an external surface of the body, which may include a vaginal or anal orifice. The topical composition may be administered in a spray, coat, soak, powder, spread, or the like, for example, suitable to the topical format.

In some embodiments, the topical composition comprises a nail lacquer for direct application to nail tissue. A nail lacquer format may include, for example, one or more antimicrobial actives formulated for topical application to nail tissue. In some embodiments, a nail lacquer format may include additives such as thickening agents, plasticizers, polymers, volatile organic compounds, or other additives to promote effective localization of the medication following application. In some embodiments, a nail lacquer format may comprise a solution, which may be a suspension or mixture. In some embodiments, a nail lacquer format may lack traditional lacquer additives. In various embodiments, a nail lacquer format may comprise an aqueous solution formulated to be applied to a nail surface whereon the carrier or component thereof evaporates or is absorbed. In some embodiments, a nail lacquer solution may have a fluid or semi-fluid consistency. In some embodiments, a carrier for a nail lacquer format may be thickened with a viscosity agent to increase viscosity for administration. In some embodiments, a nail lacquer format may comprise a solution comprising a cream, lotion, gel, or ointment.

### Methods of Treatment

The present disclosure also describes methods of topically treating conditions such as infections, pain, inflammation, itching, fluid buildup or accumulation, or other condition by providing or administering a topical composition described herein. The method may include contacting the topical composition to affected or adjacent body surfaces such as skin, nails, or body orifices. With respect to infections, the infection may be of an exterior surface of the body, an orifice, or internal. Administration may include bath-irrigation, topical irrigation via a syringe, administration in a topical powder, or a topical gel, cream, ointment, or lotion. Administration may be to an external surface of the body or to anal or vaginal surfaces. In various embodiments, the topical composition may be administered via contact to an affected area such as to skin of a head, face, ears, nose, neck, shoulder, torso, chest, stomach, waistline, extremity, arm, hand, finger, nail, groin, buttock, leg, foot, or toe, for example. In an embodiment of a method to treat an internal infection, the topical composition may be administered topically as described herein wherein one or more active agents are transdermally delivered locally or for systemic circulation. In some embodiments, the topical composition is configured as a suppository, such as a rectal, urethral, or vaginal suppository, or may be formed into a topical composition to be applied at a body orifice.

In various embodiments, the topical composition may be administered 1 to 2 times daily or as otherwise needed. In various embodiments, the topical composition comprises one or more antifungals described herein and may be administered to a body surface to treat an infection or reduce an opportunistic infection from one or more fungi selected from Aspergillus flavus, Aspergillus fumigatus, Aspergillus niger, Aspergillus terreus, Blastomyces dermatitidis, Candida species, Candida glabrata or krusei, Coccidioides immitis, Cryptococcus neoformans, Fusarium species, Histoplasma capsulatum, Leishmania donovani, Leishmania infantum, Paracoccidioides brasiliensis, Scedosporium apiospermum, Sporothrix schenckii, Trichophyton sp., and/or Trichophyton rubrum. The topical composition may comprise one or more additional actives such as antibacterials, antivirals, nonsteroidal anti-inflammatory drugs (NSAIDs), local anesthetics, keratolytics, statins, antidepressants, anticonvulsants, steroids, anesthetics, acid reducers, calcium channel blockers, antianxiety drugs, mucolytics, antihistamines, NMDA (N-Methyl-D-aspartate) receptor antagonists, muscle relaxants, anticholinergics, stimulants, or combinations thereof. In various embodiments, the topical composition comprises one or more antibacterials described herein and may be administered to a body surface to treat an infection or reduce an opportunistic infection from one or more bacteria selected from Bacteroides fragilis, Clostridium difficile, Clostridium perfringens, Chlamydia pneumoniae, Chlamydia psittaci, Chlamydia trachomatis, Mycoplasma pneumoniae, Acinetobacter baumannii, Acinetobacter calcoaceticus, Acinetobacter lwoffii, Bordetella pertussis, Brucella species, Campylobacter jejuni, Citrobacter freundii, Enterobacter aerogenes, Enterobacter cloacae, Enterobacter sakazakii, Escherichia coli, Francisella tularensis, Haemophilus ducreyi, Haemophilus influenzae, Klebsiella (Calymmatobacterium) granulomatis, Klebsiella oxytoca, Klebsiella pneumoniae, Legionella pneumophila, Moraxella catarrhalis, Morganella morganii, Neisseria gonorrhoeae, Neisseria meningitidis, Proteus mirabilis, Proteus vulgaris, Providencia sp., Pseudomonas aeruginosa, Rickettsiae, Salmonella typhi, Serratia marcescens, Shigella sp., Vibrio cholerae, Yersinia pestis, Corynebacterium jeikeium, Corynebacterium urealyticum, Enterococcus faecalis, Enterococcus faecium, Methicillin Resistant Staph aureus (MRSA), Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The topical composition may comprise one or more additional actives such as antifungals, antivirals, nonsteroidal anti-inflammatory drugs (NSAIDs), local anesthetics, keratolytics, statins, antidepressants, anticonvulsants, steroids, anesthetics, acid reducers, calcium channel blockers, antianxiety drugs, mucolytics, antihistamines, NMDA (N-Methyl-D-aspartate) receptor antagonists, muscle relaxants, anticholinergics, stimulants, or combinations thereof.

In one embodiment, a topical composition may be used to treat an infection or suspected infection accompanying a hyperkeratotic skin condition marked by a thickening of the outer layers of skin. Methods of using the topical composition may include treating an individual in need by topically applying the composition to affected skin. Conditions treated may include conditions such as those marked by thickening of the skin, referred to as hyperkeratosis. The compounded topical composition described herein may thus be applied to such affected areas of the skin to treat the affected area. The composition may alleviate symptoms such as redness, swelling, or itching. The composition may accelerate the healing process with respect to the affected skin. In various embodiments, the topical composition may be administered to treat hyperkeratotic conditions. The hyperkeratotic skin condition treated may include chronic eczema, corns, calluses, warts, seborrheic keratosis, lichen planus, actinic keratosis, as examples. The hyperkeratotic skin conditions may be caused by irritation, such as physical pressure or rubbing, chemical, infection, sunlight or radiation, or inherited conditions, for example. In an embodiment, the topical composition may be administered to such affected skin in a preventative treatment regime to combat proliferation of microbial infections with respect to the thickened skin layers. In some such embodiments, the topical composition may include a keratolytic agent as described herein.

Topical compositions comprising cream, lotion, paste, ointment, and similar formats may be applied by contact to skin, or mucosal tissue with respect to anal or vaginal administration. In some embodiments, the topical composition may be formulated in a shampoo carrier for administration in a shampoo. In some formats, the composition may be administered to an infected or target area via spray, drops, wash, swab, sponge, absorbent dressing, coating (e.g., a nail lacquer), soaking, submerging, footbath, instillation or irrigation. Embodiments comprising a nail lacquer formulation may be applied directly to nails, to treat a bacterial or fungal nail infection.

Various embodiments comprising a solution format may be administered in a footbath, which may include a handbath or soak, to treat or prevent an infection. The method may include adding the topical composition to a footbath. In some embodiments, the method may include addition of a carrier or carrier component comprising an aqueous diluent. The aqueous diluent may be in addition to the carrier as described herein or may be the carrier. For example, a topical composition comprising a solution, cream, ointment, powder, gel, paste, or lotion format may be added to a footbath. Additional carrier comprising an aqueous diluent may also be added. In some embodiments, the topical composition prior to addition of the diluent comprises a concentrated topical composition, and following addition of the carrier component comprising the diluent, the topical composition comprises the percent compositions described herein. The footbath solution may be agitated and/or heated in some embodiments. A foot or a hand may contact the footbath solution in the footbath for administration of the topical composition.

A “footbath” refers to a container that can hold some volume (e.g., about 1.0 liters to about 10 liters) of a footbath solution, which may typically be an aqueous solution or suspension, and is designed to physically accommodate at least a portion of one or both feet of a subject. A footbath administration includes administration of the topical composition utilizing a footbath. A footbath may be used as a hand bath; however, smaller bathing containers may typically be utilized as hand baths. In various embodiments, footbath solutions may be utilized as hand bath solutions. A footbath can comprise several features or agents that effect various functions. For example, a footbath can comprise one or more lights or light-emitting devices, a mechanical agitation agent (e.g., one or more jets or bubble makers) to physically agitate the enclosed water, a bubble agent to create bubbles within the enclosed water, a heating agent to heat the enclosed water, a vibration agent to vibrate the enclosed water (e.g., a high frequency vibration massage), an infrared device to provide infrared light to a foot or feet of the subject, a massage agent (e.g., a roller) that provides massaging contact to at least a portion of one or both feet, a pedicure agent that can clean or contact a foot or feet with a pumice, or a combination thereof. In an aspect, a footbath can have a water fall element. In an aspect, an agitation agent or an agitator can be coupled to both a motor and the footbath. In an aspect, a footbath can comprise one or more splash guards and other spill-resistant features to ensure that the water remains enclosed within a container. A footbath may also accommodate a subject's calves, meaning that the container is “deep” so as to allow the enclosed water to contact both the feet and at least a portion of the calves of the subject. Several manufacturers market footbaths including PIBB, Dr. Scholl's, Kendal, Conair (e.g., Model FB5X, FB3, FB27R, FB30, FB52, etc.), and Brookstone.

In various embodiments, the topical composition may be formulated for the treatment of one or more ailments of the respiratory tract, such as infections of the lower or upper respiratory tract. Respiratory tract infections treated may include a condition caused by or associated with a bacterial, viral, or fungal infection, including a Pseudomonas infection, Candida infection, Candida albicans infection, Pseudomonas aeruginosa infection, or a MRSA infection, influenza or the common cold. Other conditions treated may include cystic fibrosis, bronchiolitis, bronchiectasis, tracheobronchitis, pneumonia (including ventilator-associated pneumonia, pneumonitis, dyspnea, cough, (recurrent) wheezing, asthma, nasal polyopsis, allergic rhinitis, upper respiratory infections (Common cold), pulmonary sarcoidosis, anosmia, olfactory (smell) loss, sinus ostia stenosis, aspergilliosis, pulmonary invasive fungal infections, sinusitis, chronic rhinosinusitis, nosocomial lung infections. Pulmonary administration of the topical composition may also be employed for systemic administration of the active agents therein. Active agents administered to the respiratory tract may include those described herein, e.g., an antibacterial, antifungal, antiviral, NSAID, keratolytic, antidepressant, anticonvulsant, local anesthetic, steroid, statin, acid reducer, calcium channel blocker, antianxiety drug, mucolytic, and/or antihistamines.

The topical composition for administration to the respiratory tract may be formulated as a dry powder comprising one or more compounded active agents. In one embodiment, the dry powder may be encapsulated to provide an encapsulated dosage form, which, in some embodiments, may comprise a unit dose or unit dose form. The encapsulated dosage form may be suitable for administration to the patient or may be configured for further manipulation to produce an administration dosage form, e.g., by addition of the dry powder to a liquid. For example, in one embodiment, a first dosage form comprising the dry powder is formulated to be added to a base to produce a second administration dosage form suitable to be administered to the patient for topical application or local action at an infection site, such as an infected respiratory surface. In various embodiments, the administration dosage form comprises a mixture, suspension, emulsion, or solution, for example, prepared from the dry powder and a base. In some embodiments, the base may comprise an aqueous solution, which may include a diluent, solvent, or liquid medium.

According to one method, the topical composition is formulated for administration by mixing the dry powder with an aqueous base such as water for injection or a suitable sodium chloride solution, e.g., a 0.9% sodium chloride solution, just prior to administration, which may include the dry powder mixed, dissolved, suspended, or dispersed within the base. Administration may be by inhalation of the dry powder or by nebulization in a nebulizer solution or suspension. The nebulizer dosage form may comprise a unit dose quantity of active ingredients within a suitable volume of carrier, such as is suitable for administration via in a small volume nebulizer, which may be an intranasal nebulizer, or in a large volume nebulizer. Nebulization may be by a suitable commercially available nebulizer device, preferably an intranasal nebulizer. Nebulization delivery systems may beneficially avoid side effects associated with cold medicated irrigations or aerosol-generating powered nasal irrigators. In one embodiment, the topical composition may include or be mixed with a carrier for small particle nebulization using a small particle nebulization delivery system. For example, the nebulizer dosage form may be formulated for inhalational delivery to the lungs via a small particle size nebulizer, such as a PARI or Sinustar™, Omron, or other nebulizer configured to nebulize the dosage form to produce particles or droplets less than 10 microns in size thereby allowing penetration into the lower respiratory tract, e.g., the lungs. In one embodiment, the small particle nebulization delivery system may be configured to nebulize the formulation to produce small particles or droplets, e.g., having aerosol characteristics, wherein the majority of the particles or droplets formed by the nebulization are less than 5 microns. In some embodiments, 60%, 70%, 80%, or greater of the particles or droplets are less than 5 microns. In various embodiments, nebulization with a small particle nebulizer produces nebulized aerosol particles wherein the majority of particles are less than 10 microns, 8 microns, 5 microns, or 3 microns. In these or other embodiments, the nebulized particles may be produced within a particle size dispersion wherein at least 50%, 60%, 70%, 80%, 90%, or 95% of the particles may be within about 3 microns and about 10 microns, about 3 microns and about 8 microns, about 3 microns and about 5 microns, about 5 microns and about 8 microns, about 5 microns and about 10 microns, or about 8 microns and about 10 microns. The nebulized small particles may be inhaled into the upper airway and deposit thereafter at the paranasal sinus and nasal mucosa.

In one embodiment, the topical composition comprises a nebulizer dosage form for large particle nebulization using a large particle nebulization delivery system. In some instances, a nebulizer dosage form may be suitable for both large and small particle nebulization. Compared to large particle nebulization delivery systems, small particle nebulization delivery systems deliver a greater fraction of drug to the pulmonary system. This may increase systemic bioavailability of the active agents or drugs. In at least one embodiment, treatment of an upper or lower respiratory tract infection comprises nebulizing the nebulizer formulation to produce both small and large particles to target desired respiratory surfaces. In various embodiments, the nebulizer dosage form may be configured to be delivered by large particle nebulization intranasally via a large particle nebulizer. The large particle nebulization delivery system may produce particles or droplets of the solution wherein a majority of such particles or droplets are larger than about 5 microns, about 10 microns, about 15 microns, about 20 microns or more, such as about 23 microns. In these or other embodiments, the nebulized particles may be produced within a particle size dispersion wherein at least 50%, 60%, 70%, 80%, 90%, or 95% of the particles may be within about 10 microns and about 25 microns, about 10 microns and about 20 microns, about 10 microns and about 15 microns, about 15 microns and about 25 microns, about 15 microns and about 20 microns, or about 20 microns and about 25 microns. Accordingly, a method of administering such a formulation may comprise using a large particle nebulization system and nebulizing the formulation to form large particles. The nebulized large particles of the formulation may then be inhaled intranasally into the nasal and paranasal sinus cavities for deposition on the frontal recess/sinus, spheno-ethmoid recess, ethmoid cavity, sphenoid and maxillary sinuses, turbinates, middle meatus, and olfactory cleft. The large particles nebulization system may provide low volume, high concentration delivery of the formulation. While any suitable large particle nebulization system or device may typically be used to nebulize the formulation for administration to the patient, one suitable device is a NasoNeb® Nasal Nebulizer. The large particle delivery system may be employed to administer the formulation to the patient to deliver a deep, penetrating aerosol to the nasal and paranasal sinus cavities of the patient. Such delivery may include little to no incidental pulmonary delivery of drugs, which may otherwise occur in small particle systems as described above. For example, in some embodiments, large particle nebulization may provide superior outcomes compared to small particle nebulization, which typically include pulmonary delivery and decreased nasal and paranasal sinus cavity disposition. In one embodiment, the large particle nebulization system is configured to deliver the topical composition in a low volume carrier to ensure that the medication of the formulation stays in the nasal cavity or is deposited on respiratory surfaces in high concentration droplets. In one embodiment, the large particle nebulization system is configured to deliver between about 0.2 mL to about 15 mL of the composition for retention in the nasal and paranasal sinus cavities. In one embodiment, the large particle nebulization system may also reduce complications associated with repeated exposure to cold fluid irrigation such as exostoses of the paranasal sinus cavities by warming the composition to near room temperature upon nebulization, which may help to avoid the iatrogenic complication of exostoses from cold fluid irrigation.

According to various embodiments, the topical composition for administration to the respiratory tract includes an antifungal agent selected from nystatin, ciclopirox, amphotericin B, flucytosine, and terbinafine. In further examples, the antifungal agent may also include one or more azoles selected from clotrimazole, econazole, oxiconazole, ketoconazole, miconazole, fluconazole, itraconazole, amorolfineitraconazole, and voriconazole. In one example, the topical composition comprises flucytosine together with an above azole. In another example, flucytosine is provided without an azole. According to various embodiments, topical composition comprises a dry powder formulation or a solution or suspension including the dry powder formulation in a carrier. The topical composition may include additional active agents such as an antibiotic, e.g., aminoglycoside such as kanamycin, tobramycin, gentamicin, or neomycin, and/or a mucolytic agent, such as acetylcysteine. Additional active or inactive agents may include anesthetics, analgesics, anti-inflammatories, or emollients. The topical composition may include one or excipients formulated to assist in the release, dispersion, solubility, or the delivery of one or more of the active agents at the administration or target site, e.g., lungs, respiratory surfaces, or one or more body orifices. For example, excipients may include synthetic or naturally derived excipients such as one or more of a base, solvent, surfactant, permeation enhancer, emollient, humectant, disintegrate, acid, base, pH modifying agent or buffer, solubility enhancer, or a carrier molecule or complex configured to enhance or modulate diffusion, localization, targeting, active or passive transport, or uptake, for example, of one or more active agents, such as the aminoglycoside.

In some embodiments, the topical composition may be utilized in a combinational treatment format. For example, all or a portion of the carrier may be combined with all or a portion of the active agent at a body surface, e.g., skin or mucosal surface. For example, powder comprising all or a portion of an active agent may be applied to a body surface. In further or another example, all or a portion of the active agent may be applied to a body surface within a liquid comprising a first portion of the carrier as a solution, suspension, or dispersion. The body surface may correspond to an area to be treated by the active. While the active agent is contacting the body surface, a carrier or second portion of the carrier may be applied to the body surface to combine with the active agent. The carrier may mix or be actively mixed with the active at the body surface. In some formulations, the carrier may provide occlusive functionalities. In one example, the carrier or second portion of the carrier comprises a solution, suspension, dispersion, gel, ointment, cream, lotion, emulsion, or foam. In one embodiment, the carrier or second portion of the carrier comprises a water washable ointment such as Bassa-Gel. In various embodiments, the topical composition comprises a gel, ointment, lotion, or cream and the method comprises soaking the target body surface with an aqueous solution, such as water, followed by contacting the body surface with the topical composition without drying the body surface. The aqueous solution may also be a dispersion, mixture, and/or suspension. Soaking may comprise submerging or maintaining continuous contact between the solution and the body surface. Soaking may be for about 5 minutes to about 60 minutes, such as between about 5 minutes and about 30 minutes, about 10 minutes and about 30 minutes, about 15 minutes and about 30 minutes, or about 15 minutes and about 25 minutes. In one embodiment, the aqueous soaking solution comprises a topical composition described herein including an active agent combined with an aqueous carrier and the gel, ointment, lotion, or cream comprises a second carrier alone or with a second portion of the active agent. In one example the first and second portions of the active agent comprise a same active. While the body surface will typically not be dried between soaking and contact with the gel, ointment, lotion, or cream, in some embodiments, the body surface may be dried before contact with the gel, ointment, lotion, or cream. In one embodiment, the carrier or second portion of the carrier comprises a water washable ointment such as Bassa-Gel.

### Method of Compounding a Topical Composition

In various embodiments, a method of formulating the topical composition described above or elsewhere herein comprises combining the one or more active agents and a topical carrier. In some embodiments, the method may include formulating the carrier separately or together with the combining of the one or more active agents.

A method of formulating a topical composition may include combining one or more active agents, such as any active agent described herein, e.g., an antibacterial, antifungal, antiviral, NSAID, keratolytic, antidepressant, anticonvulsant, local anesthetic, steroid, statin, acid reducer, calcium channel blocker, antianxiety drug, mucolytic, and/or antihistamine, and a carrier. In some embodiments, the active agent may include pure powder, vials, and/or capsule formats of one or more actives combined with the carrier in addition to or instead of powder from crushed tablets. Combining may include adding all or a portion of the powder to be combined with all or a portion of the carrier and mixing. In some embodiments, all or a portion of the powder may be dispersed, suspended, or dissolved in a liquid to form a paste, solution, dispersion, or suspension prior to addition to the carrier. In one of an above or another embodiment, all or a portion of the powder may be directly added to all or a portion of the carrier. Combining may include blending the carrier and active to formulate a smooth composition. In some embodiments, the active agent or a portion thereof may dissolve or solubilize in the carrier when combined. According to various embodiments, the carrier may comprise a suitable carrier selected to formulate a topical composition comprising a format selected from a cream, gel, lotion, ointment, emulsion (oil-in-water or water-in-oil), foam, solution, dispersion, or powder, for example, suitable for topical application. The carrier may be formulated together or separately from combining with one or more active agents. The carrier may be a pre-formulated composition. The carrier may be present in an amount sufficient to obtain the desired amount of active agents per unit weight or volume. The one or more active agents may be mixed dispersed, suspended, solubilized, or dissolved with the carrier.

In some embodiments, the method includes combining a first portion of the active agent comprising capsules and/or crushed tablets with a second portion of the active agent. The method may include combining the first and second portions and the carrier together or separately. The active agent may be one or more antifungal agents, one or more antibacterial agents, one or more antiviral agents, one or more NSAID agents, one or more keratolytic agents, one or more antidepressant agents, one or more anticonvulsant agents, one or more local anesthetic agents, one or more steroid agents, one or more statin agents, one or more acid reducer agents, one or more calcium channel blocker agents, one or more antianxiety agents, one or more mucolytic agents, one or more antihistamine agents, or a combination thereof. In one embodiment, the second portion of the active agent comprises a commercially available medicated composition.

As introduced above, the method may include combining active agents in addition to the active agent. In some embodiments, the additional active agent comprises one or more active agents selected from an antibacterial agent, an antifungal agent, an antiviral agent, an anti-inflammatory agent, a steroid, an anti-allergy drug or antihistamine agent, a calcium channel blocker, an antidepressant agent, a stimulant agent, a disinfectant agent, an anticonvulsant agent, a local anesthetic agent, mucolytic, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof.

It will be appreciated that topical compositions herein may include or specifically exclude an additional active agent. It will also be appreciated that topical compositions may exclude an active agent and rather include one or more of the additional active agents described herein. Some embodiments of the topical compositions may exclude biologics.

The method of formulating the topical composition comprising additional active agents may include combining all or a portion of an additional active agent from a powder format, e.g., from bulk powder, crushed tablet, injection powder, or other commercially available composition. In various embodiments, such additional active agents may be combined with the carrier together with or separate from all or a portion of the active agent powder or other format. According to a method, all or a portion of an additional active agent may be provided in a format selected from a solution, emulsion, gel, cream, lotion, ointment, or other format and may be combined with the carrier together with or separate of all or a portion of the active agent. In one example, all or a portion of the additional active agent may be mixed with all or a portion of the active agent prior to being added to the carrier. In another example, the active agent is added to the additional active agent that is provided in a commercially available medicated composition comprising the carrier.

In one aspect a method of delivering the topical composition described herein includes delivery to an upper or lower respiratory tract of a subject. The topical composition may include a dry powder or a dry powder mixed with an aqueous diluent or carrier to formulate a solution or suspension. The topical composition may include one or more active agents described herein, such as one or more antibacterials, antifungals, antivirals, local anesthetics, antidepressants, steroids, NSAIDs, statins, keratolytics, acid reducers, calcium channel blockers, antianxiety drugs, mucolytics, or antihistamines, including combinations thereof, in an amount between about 0.001% and about 50% by weight, which is to be understood to include any weight or range of weights therebetween, such as any weight or range or weight or percent composition disclosed herein. In an above or another embodiment, the method comprises combining one or more identified antifungals in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight, such as any weight or weight range or percent composition disclosed herein. The method may further comprise encapsulating a dry powder formulation including the active agent in a capsule and dispensing the encapsulated dry powder formulation for subsequent addition of the dry powder formulation to water for injection, sodium chloride solution, or other suitable dilutant or carrier to produce the nebulizer solution dosage form for administration to the patient via nebulization, such as intranasal nebulization, to treat the infection of the upper or lower respiratory tract and/or for pulmonary drug delivery.

In one embodiment, formulating the topical composition comprising combining the active agent with a commercially available medicated composition comprising all or a portion of the additional active agent.

The method may include combining the carrier and powder containing all or a portion of one or more of the active agents. For example, one or more actives may be obtained from bulk pure powder or powder for injection and combined with the carrier. In one of an above or another example, one or more actives may be obtained from one or more commercially available oral tablets. The oral tablets may be crushed and the resulting powder may be combined with the carrier. In one embodiment, the active agent comprises an antifungal agent selected from fluconazole, voriconazole, or combination thereof. In one embodiment, the method includes crushing voriconazole 50 mg, 100 mg, and/or 200 mg tablets to obtain a powder and/or combining powder from crushed voriconazole 50 mg, 100 mg, and/or 200 mg tablets and the carrier. In this or another embodiment, the method includes crushing fluconazole 100 mg and/or 200 mg tablets to obtain a powder and/or combining powder from crushed fluconazole 100 mg and/or 200 mg tablets and the carrier. As a further or another example, the active agent comprises an antibacterial agent selected from levofloxacin, ciprofloxacin, linezolid, or combination thereof. In one embodiment, the method includes crushing linezolid 600 mg tablets to obtain a powder and/or combining powder from crushed linezolid 600 mg tablets and the carrier. In this or another embodiment, the method includes crushing levofloxacin 250 mg, 500 mg, and/or 750 mg tablets to obtain a powder and/or combining powder from crushed levofloxacin 250 mg, 500 mg, and/or 750 mg tablets and the carrier. In an above or another embodiment, the method includes crushing ciprofloxacin 250 mg, 500 mg, and/or 750 mg tablets to obtain a powder and/or combining powder from crushed ciprofloxacin 250 mg, 500 mg, and/or 750 mg tablets and the carrier.

The number of tablets to be crushed to formulate a topical composition comprising a desired weight of an active agent may be determined by dividing the weight of the active agent needed by the weight of the active in a tablet. The weight of crushed tablet powder needed to formulate a topical composition comprising a desired weight of an active agent may be determined by dividing the weight of a tablet by the weight of the active agent present in the tablet and multiplying the result by the desired weight of the active agent in the topical composition.

In some embodiments, one or more commercially available oral capsules may be used in the formulation of the topical composition. For example, one or more oral capsules may be opened and their contents combined with the carrier. In one formulation, the active agent comprises or consists of an antifungal agent comprising or consisting of flucytosine. In one example, the method includes emptying the contents of one or more flucytosine capsules, such as 250 mg or 500 mg capsules, USP, for combining with the carrier. In some instances, the capsule itself may be mixed with the carrier wherein the capsule dissolves, solubilizes, and/or breaks up; however, the one or more capsules may be preferably opened to release the contents for combining the contents with the carrier. In some embodiments, one or more actives may be contained in a compounded capsule. For example, the topical composition may be formulated by combining powder contents of a compounded capsule including one or more actives with the carrier. The number of capsules to formulate a topical composition comprising a desired weight of an active agent may be determined by dividing the weight of the active agent needed by the weight of the active in a capsule. The weight of capsule powder needed to formulate a topical composition comprising a desired weight of an active agent may be determined by dividing the weight of a capsule or contents by the weight of the active agent present in the capsule and multiplying the result by the desired weight of the active agent in the topical composition.

In addition to actives, in various embodiments, the powder from one or more a capsules and/or crushed tablets may include one or more of a glucose polymer, starch, and/or cellulose and one or more additional components such as magnesium stearate, povidone, lactose, glycol, oxide, talc, triacetin, or an alcohol. In some embodiments, the powder includes a cellulose such as microcrystalline cellulose and one or more of magnesium stearate, anhydrous dibasic calcium phosphate, or povidone. In one example, the powder may further include croscarmellose sodium. In a further example, the powder may also include an oxide such as silicon dioxide, ferric oxide, aluminum oxide, or combination thereof; a starch, such as sodium starch glycolate, corn starch, or both; sodium lauryl sulfate, lactose, talc, or combinations thereof. In some embodiments, wherein a tablet includes a film coating, the method may include removal of all or a portion of a film coating.

In some embodiments, the method includes formulating a carrier and/or combining active agent with a carrier comprising a base cream, ointment, gel, lotion, foam solution, or powder. The carrier may include lecithin, phospholipids, glycols, paraffin, fatty acids, carbopols/carbomers, alcohols, lanolin, or combination thereof, for example. In one embodiment, the carrier comprises a sodium chloride 0.09% solution (sterile). Some embodiments may include polyethylene glycol (PEG), while other embodiments may be PEG-free. In an above embodiment or another embodiment, the carrier may include a silicon or silicon variant or may be silicon-free. A carrier solution may comprise an aqueous or non-aqueous solution. Example solutions may include water, alcohol, DMSO, saline or sodium chloride, and/or sodium hypochlorite. In some embodiments, the carrier comprises an aqueous solution such as a saline solution. For example, the topical composition may comprise a carrier comprising sodium chloride solution, which may be a sterile solution, an alcohol, water, e.g., purified water, water for irrigation, water for injection, or sterile water. The carrier may be water soluble/miscible or formulated for water absorption, such as a gel.

In some embodiments, the method includes combining active agent with a carrier to formulate a topical composition comprising a water-in-oil emulsion or oil-in-water emulsion. For example, the carrier may comprise an emulsion having a cream or lotion format including one or more of acrylate copolymers, alcohol, camphor, carbomer, dimethyl isosorbide, disodium EDTA, dl-alphatocopheryl acetate, edetate disodium, emulsifying wax, Eucalyptus oil, flavonoids, glycerin, glycol dicaprylate/dicaprate, hydroxyethyl cellulose, isopropyl myristate, lactic acid, meadowsweet extract, menthol, mineral oil, neopentyl, phenolic glycosides, polyethylene glycol (PEG), polysorbate (e.g., polysorbate 85, polysorbate 20), purified water, titanium dioxide, tridecyl stearate, tridecyl trimellitate, sodium hydroxide, sodium hydroxide, sorbitol, stearic acid, zinc pyrithione, or combinations thereof.

In some embodiments, the method includes combining active agent with a carrier to formulate a topical composition comprising an ointment format. For example, the carrier may comprise hydrophilic petrolatum, white petrolatum, hydrophilic ointment, white ointment, anhydrous lanolin, hydrous lanolin, PEG ointment, or combinations thereof. In some embodiments, the method includes combining the active agent with a carrier to formulate a topical composition comprising a gel. The gel may be an aqueous or non-aqueous gel. The gel may include thickening agents and/or gelling agents such as carbopol, poloxamer, xanthan gum, methylcellulose, carboxymethyl cellulose, hydroxyethyl cellulose, ethylcellulose, gelatin, magnesium aluminum silicate, polyvinyl alcohol, sodium alginate, or combinations thereof. In some embodiments, the method includes combining the active agent with a carrier to formulate a topical composition comprising a powder. A powder carrier may include lactose or talc, for example. In some embodiments, the method may include imparting the carrier or topical composition formulated therewith with a gas or pressurized propellant to generate a foam format. For example, a propellant such as butane may be used to generate a foam from the carrier or combined carrier and active agent. In various embodiments, the method may include utilizing a carrier or further combining of one or more carrier component additives such as solubilizers, stabilizers (which may include antioxidants), buffers, tonicity modifiers, bulking agents, viscosity enhancers/reducers, surfactants, chelating agents, adjuvants, humectants, preservatives, flavorings, binders, colorants, or combinations thereof.

Further to the above, in some embodiments, the method includes combining active agent with a commercially available carrier or base vehicle composition for compounding. The carrier may be liquid, semi-liquid, or solid. For example, the carrier may include an aqueous, organic, or inorganic solution, which may include a dispersion or suspension, cream, gel, ointment, lotion, emulsion, powder, or paste. Thus, the method of formulating the topical composition may include addition of crushed antimicrobial tablets or powder thereof to a topical base for compounding to formulate creams, ointments, solutions/irrigations/baths, powders, gels, lotions, or pastes, for example. Non-limiting examples may include Spira-Wash® Gel, Lipoderm®, Loxasperse®, Mucolox™, or Versabase® Cream, Goam, Gel, Lotion or Shampoo, manufactured and distributed by PCCA, 9901 South Wilcrest Drive, Houston, Tex. 77099.

In some embodiments, the method includes combining one or more actives with a carrier comprising a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel.

In one embodiment, the method of formulating the topical composition comprises combining all or a portion of one or more active agents and a carrier comprising a commercially available medicated composition. In one example, the method includes combining all or a portion of an active agent comprising one or more capsules and/or crushed tablets and commercially manufactured medicated composition. The commercially manufactured medicated composition may comprise a cream, ointment, lotion, suspension, dispersion, solution, irrigation, bath, powder, gel, foam, paste, for example. Thus, a method of formulating the topical composition may comprise adding a first portion of the active agent comprising pure powder, capsules, and/or crushed tablets to a medicated composition comprising a second portion of the active agent and at least a portion of the carrier. The commercially available composition may comprise a medicated composition for oral administration, topical administration, ophthalmic administration, otic administration, nasal administration, transdermal administration, sublingual administration, or pulmonary administration.

In an embodiment, the method of formulating the topical composition includes combining capsules and/or crushed tablets of a portion of the active agent to another portion of the active agent comprising another format, such as a commercially available medicated suspension, solution, ointment, cream, gel, lotion, or powder. In some such embodiments, the carrier may be provided by the commercially available medicated composition. In various embodiments, all or a portion of the powder may be added to one or more components of the carrier. Thereafter, additional carrier components and the remainder of the powder may be added to formulate the topical composition. Other active agents may also be added before, after, or with the powder. It will be appreciated that unless indicated otherwise, combining or adding the powder to the carrier or component thereof may include combining or adding the powder after the powder has been combined or mixed with a liquid or other composition. In one example, combining the powder and carrier results in a formulation of a topical composition having a different format than that of the carrier, such a commercially manufactured base for compounding or commercially manufactured medicated composition. Examples may include addition of additional carrier components such described above such as thickening agents, thinners, surfactants, carbomers, PEG, hydrocarbons, and/or diluents.

In various embodiments, the method of formulating the topical composition may comprise combining with a carrier and one or more antifungal drugs, such as any of the antifungals described herein in an amount between about 0.01% and about 50% by weight of the topical composition. The active agent may be combined with the carrier to formulate creams, ointments, solutions/irrigations/baths, powders, gels, lotions, nebulizer solutions or suspensions, or pastes, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. In one example, the method includes combining an active agent comprising one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. The antifungal drugs may comprise tablets, capsules, vials for injection or solution, bulk power, solutions, topical ointments, creams, lotions, and/or gels, for example. In one example, the antifungal comprises or consists of flucytosine in a dosage amount between 10 mg and 1500 mg, or other amount described herein. In one embodiment, the method of making the topical composition comprises combining with the carrier one or more antifungals selected from fluconazole, itraconazole, voriconazole, amphotericin, nystatin, clotrimazole, econazole, ketoconazole or combinations thereof. In one embodiment, the method of making the topical composition comprises combining with the carrier one or more antifungals selected from fluconazole, griseofulvin, ketoconazole, metronidazole, voriconazole, itraconazole, clotrimazole, and combinations thereof. The method of formulating the topical composition may also include combining on or more additional portions of the active agent selected from one or more herein identified antibacterials, antifungals, antivirals, NSAIDs, keratolytics, statin, antidepressants, anticonvulsants, steroids, local anesthetics, acid reducers, calcium channel blockers, antianxiety drugs, mucolytics, antihistamines, stimulants, disinfectants, nerve depressants, muscle relaxants, NMDA (N-Methyl-D-aspartate) receptor antagonists, opiate or opioid agonists, anticholinergics and/or other active agents. The topical composition may be administered to the skin, vagina, nasal.

In one embodiment, a second portion of the active agent may comprise all or a portion of an antifungal agent selected from any of the antifungal agents described herein. For instance, a second portion of the active agent may comprise all or a portion of an antifungal agent comprising an azole. In one example, the second portion of the antifungal agent may comprise voriconazole bulk powder. In another or further example, a second portion of the antifungal agent may comprise fluconazole bulk powder or powder for suspension. In another or further example, a second portion of the antifungal agent may comprise flucytosine bulk powder or contents of one, more, or a fraction of flucytosine capsules. In an above or another example, a second portion of the antifungal agent may comprise itraconazole, ketoconazole, econazole, clotrimazole, fluconazole, metronidazole, amphotericin, or combination thereof.

In one embodiment, the carrier may comprise a commercially manufactured medicated composition comprising a second portion of the active agent comprising all or a portion of an antifungal agent selected from one or more of the antifungal agents described herein, such as an azole. The composition may comprise, for example, a commercially available medicated composition comprising an antifungal suspension, solution, dispersion, ointment, cream, gel, lotion, or powder.

In one embodiment, the method includes combining an active agent comprising one or more antifungals comprising amphotericin and the carrier. The amphotericin may be combined with the carrier to formulate emulsions, lotions, creams, ointments, gels, shampoos, nail lacquers, solutions, suspensions, dispersions, irrigations/baths, or pastes, for example. The amphotericin may comprise commercially available tablets, capsules, vials for injection or solution, bulk power, solutions, topical ointments, creams, lotions, and/or gels, for example. In some embodiments, amphotericin comprises amphotericin B injection, Lipid Complex; Amphotericin B injection, powder, lyophilized, for Solution; or bulk powder. For example, a method of formulating the topical composition may comprise combining the contents of one or more 50 mg Amphotericin B Injection, Powder, lyophilized for solution comprising a sterile, nonpyrogenic, lyophilized cake providing 50 mg amphotericin B and 41 mg sodium deoxycholate buffered with 20.2 mg sodium phosphates (including mono and dibasic sodium phosphate, phosphoric acid and sodium hydroxide) with a dosage volume of the carrier. Other strengths may be used. The contents of the one or more vials may be added to a volume of the carrier sufficient to formulate an administration dosage of the topical composition having the desired strength of the amphotericin B. In some embodiments, the amount of amphotericin B in an administration dosage corresponds to an amount of amphotericin B present in a vial, or a multiple thereof, such as two or three times that present in a vial.

In various embodiments, the active agent includes amphotericin, e.g., amphotericin B, and an administration dosage of the topical composition comprises amphotericin in an amount about 50 mg or less, greater than about 50 mg or greater, about 100 mg or greater, about 150 mg or greater, about 200 mg or greater, between about 50 mg and about 100 mg, between about 70 mg and about 150 mg, or between about 100 mg and about 200 mg. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available amphotericin formats such as capsules, vials for injection or solution, bulk power, solutions, topical ointments, creams, lotions, and/or gels, for example. The topical composition may be administered to the skin, vagina, nasal passage, or anal cavity. The topical composition comprising amphotericin may be applied by contact to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including amphotericin may be applied to skin in powder or solution followed by application of a carrier or second portion of the carrier comprising gel, lotion, ointment, or cream, which may include a second portion of amphotericin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including amphotericin may be administered to a body surface to treat or prevent an opportunistic infection comprising Aspergillus fumigatus, Candida species, e.g., Candida glabrata, Candida krusei.

In one embodiment, the method includes combining an active agent comprising one or more antifungals comprising econazole and the carrier. The econazole may be combined with the carrier to formulate emulsions, lotions, creams, ointments, gels, shampoos, nail lacquers, solutions, suspensions, dispersions, irrigations/baths, or pastes, for example. In various embodiments, an administration dosage of the topical composition comprises econazole in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. The econazole may comprise commercially available tablets, capsules, vials for injection or solution, bulk power, solutions, topical ointments, creams, lotions, and/or gels, for example.

In one example, the method comprises combining a first portion of the active agent with a second portion of the active agent, wherein the second portion of the active agent comprises a commercially available econazole composition such as Econazole Nitrate Cream or Econazole Nitrate Foam. The commercially available econazole composition may provide the carrier and/or additional carrier and/or carrier components described herein may be further combined. The first portion of the active agent may include one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof. In an example, the antifungal agent or a carrier comprising at least a portion of the antifungal agent may comprise a commercially available econazole such as an econazole nitrate 1.0% cream. Other strengths may be used. In an above or another example, a method of formulating the topical composition may comprise combining one or more econazole tablet, capsules, vials, and/or bulk power with a carrier. In one example, the carrier comprises a water washable, moisturizing ointment comprising polyethylene glycol, water, Spiraea ulmaria flower extract, zinc acetate, and propylene glycol. As noted above, some embodiments may include other commercially available econazole formats such as capsules, vials for injection or solution, bulk power, solutions, topical ointments, creams, lotions, and/or gels, for example. In one embodiment, the polyethylene glycol comprises PEG-8 and PEG-75 and the carrier comprises Bassa-gel. The topical composition may be administered to the skin, vagina, nasal passage, or anal cavity.

In one embodiment, the method includes combining an active agent comprising one or more antifungals comprising fluconazole and the carrier. The fluconazole may be combined with the carrier to formulate emulsions, lotions, creams, ointments, gels, shampoos, nail lacquers, solutions, suspensions, dispersions, irrigations/baths, or pastes, for example. The fluconazole may comprise commercially available tablets, capsules, vials for injection or solution, bulk power, solutions, topical ointments, creams, lotions, and/or gels, for example. In an example, the fluconazole may comprise a commercially available fluconazole, such as Fluconazole in Dextrose Inject, Solution; Fluconazole in Sodium Chloride Injection, Solution; Fluconazole Injection; Fluconazole Powder, for Suspension; Fluconazole Tablets; or bulk powder. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the fluconazole comprises a commercially available fluconazole such as 50 mg, 100 mg, 150 mg, or 200 mg fluconazole oral tablets and a method of formulating the topical composition comprises addition of a crushed fluconazole tablet to a carrier. Less than all the powder of a crushed tablet may be used when the tablet contains more fluconazole than required. More than one crushed tablet may be used when the method includes formulating a topical composition comprising more fluconazole than is in the tablet. The fluconazole tablets may comprise commercially available fluconazole 50 mg, 100 mg, 150 mg, or 200 mg oral tablets. In some embodiments, other strength tablets may be used. In addition to fluconazole the tablet powder may include a glucose polymer and one or more additional components such as magnesium stearate, povidone, lactose, glycol, oxide, talc, triacetin, or an alcohol. In some embodiments, the powder includes a cellulose such as microcrystalline cellulose and one or more of magnesium stearate, anhydrous dibasic calcium phosphate, or povidone. In one example, the powder may further include croscarmellose sodium. In a further example, the powder may also include an oxide such as silicon dioxide, ferric oxide, aluminum oxide, or combination thereof; a starch, such as sodium starch glycolate, corn starch, or both; sodium lauryl sulfate, lactose, and talc. The oral tablets may be crushed and combined with the carrier to formulate a topical composition comprising between 0.01% and 20% by weight, such as about 1%, about 2%, about 3%, about 4%, about 5%, less than about 5%, between about 2% and about 7%, or greater than about 10% fluconazole by weight. To formulate a topical composition comprising a desired percent by weight fluconazole, the total desired weight of the topical composition is subtracted from the weight of crushed oral fluconazole tablet powder needed to obtain the desired percent by weight fluconazole in a manner similar to that described above with respect to voriconazole. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the fluconazole. In some embodiments, the amount of fluconazole in the administration dosage corresponds to the amount of fluconazole present in one tablet, or a multiple thereof.

In various embodiments, the active agent includes fluconazole and an administration dosage of the topical composition comprises fluconazole in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available fluconazole formats such as capsules, vials for injection or solution, bulk power, solutions, topical ointments, creams, lotions, and/or gels, for example. The topical composition may be administered to the skin, vagina, nasal passage, or anal cavity. The topical composition comprising fluconazole may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including fluconazole may be applied to skin in powder or solution followed by application of a carrier or second portion of the carrier comprising gel, lotion, ointment, or cream, which may include a second portion of fluconazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including fluconazole may be administered to a body surface to treat or prevent an opportunistic infection comprising Candida species, Trichophyton sp., and/or Trichophyton rubrum.

In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising a base, such as those described herein. In an above or another example, the carrier may comprise a commercially available medicated composition, such as those described herein. Additional actives may include one or more antifungals, antibacterials, steroids, keratolytics, NSAIDS, acid reducers, for example. Such additional active agent may be present in a combined amount between 0.01% and 50% by weight, such as between about 0.01% and about 5%. Additionally or alternatively, additional actives may include other active agents such as one or more active agents selected from an antiviral, anti-inflammatory, antihistamine, antidepressant, stimulant, disinfectant, anticonvulsant, local anesthetic, anticonvulsant or nerve depressant agent, muscle relaxant, NMDA receptor antagonist, opiate or opioid agonist, analgesic, or combination thereof. Such additional active agents may be present in a combined amount between 0.01% and 25% by weight, such as between about 1% and about 10%.

Topical composition comprising fluconazole may be applied by contact to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including fluconazole may be applied to skin in powder or solution followed by application of a carrier or second portion of the carrier comprising gel, lotion, ointment, or cream, which may include a second portion of fluconazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In an example, the antifungal or a carrier comprising at least a portion of the antifungal may comprise a commercially available fluconazole, such as Fluconazole in Dextrose Injection Solution; Fluconazole in Sodium Chloride Injection, Solution; Fluconazole Injection; Fluconazole Powder, for Suspension; Fluconazole Tablets; or bulk powder. In one embodiment, the method comprises combining a first portion of the active agent with a second portion of the active agent, wherein the second portion of the active agent comprises a commercially available fluconazole composition such as Fluconazole Cream, Fluconazole in Dextrose Injection Solution, Fluconazole in Sodium Chloride Injection Solution, Fluconazole Injection Solution, or Fluconazole Suspension. The commercially available fluconazole composition may provide the carrier and/or additional carrier and/or carrier components described herein may be further combined. The first portion of the active agent may include one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof.

In one embodiment, the method includes combining an active agent comprising one or more antifungals comprising griseofulvin and the carrier. The griseofulvin may be combined with the carrier to formulate emulsions, lotions, creams, ointments, gels, shampoos, nail lacquers, solutions, suspensions, dispersions, irrigations/baths, or pastes, for example. The griseofulvin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining one or more 125 mg, 250 mg or 500 mg griseofulvin oral tablets with a carrier. Each tablet may contain 250 mg or 500 mg of griseofulvin microsize and one or more of calcium stearate, colloidal silicon dioxide, corn starch, crospovidone, dibasic calcium phosphate, and sodium starch glycolate. In one example, each tablet includes ultramicrosize griseofulvin 125 mg or 250 mg and one or more of lactose monohydrate, corn starch, microcrystalline cellulose, povidone, polyethylene glycol 400, poloxamer 188, anhydrous lactose, silicon dioxide, crospovidone, or magnesium stearate. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the griseofulvin. In some embodiments, the amount of griseofulvin in the administration dosage corresponds to the amount of griseofulvin present in one tablet, or a multiple thereof.

In various embodiments, the active agent includes griseofulvin and an administration dosage of the topical composition comprises griseofulvin in an amount about 100 mg or less, about 100 mg or greater, about 150 mg or greater, about 200 mg or greater, about 250 mg or greater, about 350 mg or greater, about 450 mg or greater, about 550 mg or greater, about 650 mg or greater, about 850 mg or greater, about 1 g or greater, between about 100 mg and about 200 mg, between about 200 mg and about 300 mg, between about 300 mg and about 500 mg, or between about 500 mg and about 1 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available griseofulvin formats such as capsules, vials for injection or solution, bulk power, solutions, topical ointments, creams, lotions, and/or gels, for example. The topical composition may be administered to the skin, vagina, nasal passage, or anal cavity. The topical composition comprising griseofulvin may be applied by contact to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including griseofulvin may be applied to skin in powder or solution followed by application of a carrier or second portion of the carrier comprising gel, lotion, ointment, or cream, which may include a second portion of griseofulvin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method includes combining an active agent comprising one or more antifungals comprising ketoconazole and the carrier. The ketoconazole may be combined with the carrier to formulate emulsions, lotions, creams, ointments, gels, shampoos, nail lacquers, solutions, suspensions, dispersions, irrigations/baths, or pastes, for example. The ketoconazole may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, ketoconazole may comprise a commercially available ketoconazole such as 50 mg, 100 mg, or 200 mg tablets. Tablets may also include one or more of colloidal silicon dioxide, corn starch, lactose monohydrate, magnesium stearate, microcrystalline cellulose, and/or povidone. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the ketoconazole. In some embodiments, the amount of ketoconazole in the administration dosage corresponds to the amount of ketoconazole present in one tablet, or a multiple thereof.

In various embodiments, the active agent includes ketoconazole and an administration dosage of the topical composition comprises ketoconazole in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available ketoconazole formats such as capsules, vials for injection or solution, bulk power, solutions, topical ointments, creams, lotions, and/or gels, for example. The topical composition may be administered to the skin, vagina, nasal passage, or anal cavity. The topical composition comprising ketoconazole may be applied by contact to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including ketoconazole may be applied to skin in powder or solution followed by application of a carrier or second portion of the carrier comprising gel, lotion, ointment, or cream, which may include a second portion of ketoconazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method comprises combining a first portion of the active agent with a second portion of the active agent, wherein the second portion of the active agent comprises a commercially available ketoconazole composition such as Ketoconazole Foam, Ketoconazole Cream, Ketoconazole Suspension, or Ketoconazole Suspension Shampoo. The commercially available ketoconazole composition may provide the carrier and/or additional carrier and/or carrier components described herein may be further combined. The first portion of the active agent may include one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof.

In one embodiment, the method includes combining an active agent comprising one or more antifungals comprising metronidazole and the carrier. The metronidazole may be combined with the carrier to formulate emulsions, lotions, creams, ointments, gels, shampoos, nail lacquers, solutions, suspensions, dispersions, irrigations/baths, or pastes, for example. The metronidazole may comprise commercially available tablets, capsules, vials for injection or solution, bulk power, solutions, topical ointments, creams, lotions, and/or gels, for example. Each tablet may contain about 250 mg or about 500 mg metronidazole and one or more of colloidal silicon dioxide, crospovidone, hydrogenated vegetable oil, and/or microcrystalline cellulose. Each tablet may contain about 250 mg or about 500 mg metronidazole and one or more of colloidal silicon dioxide, hydroxypropyl cellulose, lactose (anhydrous), microcrystalline cellulose, sodium starch glycolate, and/or stearic acid. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the metronidazole. In some embodiments, the amount of metronidazole in the administration dosage corresponds to the amount of metronidazole present in one tablet, or a multiple thereof.

In various embodiments, the active agent includes metronidazole and an administration dosage of the topical composition comprises metronidazole in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available metronidazole formats such as capsules, vials for injection or solution, bulk power, solutions, topical ointments, creams, lotions, and/or gels, for example. The topical composition may be administered to the skin, vagina, nasal passage, or anal cavity. The topical composition comprising metronidazole may be applied by contact to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including metronidazole may be applied to skin in powder or solution followed by application of a carrier or second portion of the carrier comprising gel, lotion, ointment, or cream, which may include a second portion of metronidazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method includes combining an active agent comprising one or more antifungals comprising nystatin and the carrier. The nystatin may be combined with the carrier to formulate emulsions, lotions, creams, ointments, gels, shampoos, nail lacquers, solutions, suspensions, dispersions, irrigations/baths, or pastes, for example. In one embodiment, the method includes combining nystatin with the carrier wherein the nystatin comprises a commercially available nystatin, such as Nystatin Powder (Topical), or bulk powder. In various embodiments, an administration dosage of the topical composition comprises Nystatin in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include commercially available nystatin formats such as tablets, capsules, vials for injection or solution, bulk power, solutions, topical ointments, creams, lotions, and/or gels, for example. The topical composition may be administered to the skin, vagina, nasal passage, or anal cavity.

In one embodiment, the method comprises combining a first portion of the active agent with a second portion of the active agent, wherein the second portion of the active agent comprises a commercially available nystatin composition such as Nystatin Cream or Nystatin Ointment. The commercially available nystatin composition may provide the carrier and/or additional carrier and/or carrier components described herein may be further combined. The first portion of the active agent may include one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof.

In one embodiment, the method includes combining an active agent comprising one or more antifungals comprising itraconazole and the carrier. The itraconazole may be combined with the carrier to formulate emulsions, lotions, creams, ointments, gels, shampoos, nail lacquers, solutions, suspensions, dispersions, irrigations/baths, or pastes, for example. The itraconazole may comprise commercially available tablets, capsules, vials for injection or solution, bulk power, solutions, topical ointments, creams, lotions, and/or gels, for example. In an example, the antifungal agent or a carrier comprising at least a portion of the antifungal agent may comprise a commercially available itraconazole, such as an Itraconazole Capsule; Itraconazole Injection Solution; or bulk powder. For example, an itraconazole capsule, e.g., 100 mg capsule, or contents thereof, including itraconazole coated in sugar spheres comprising sucrose, starch, and water, may be added to a dosage volume of the carrier. In one embodiment, itraconazole bulk powder, which may be included in a compounded capsule that may be opened by a user for addition to the carrier, may be used. In one example between about 25 mg and about 100 mg, such as about 50 mg, may be combined with a dosage volume of the carrier. Other strengths may be used.

In various embodiments, the active agent includes itraconazole and an administration dosage of the topical composition comprises itraconazole in an amount about 50 mg or less, about 50 mg or greater, about 100 mg or greater, about 150 mg or greater, about 200 mg or greater, about 300 mg or greater, about 400 mg or greater, about 500 mg or greater, between about 50 mg and about 200 mg, between about 200 mg and about 300 mg, or between about 300 mg and about 550 mg. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include commercially available itraconazole formats such as tablets, capsules, vials for injection or solution, bulk power, solutions, topical ointments, creams, lotions, and/or gels, for example. The topical composition may be administered to the skin, vagina, nasal passage, or anal cavity. The topical composition comprising itraconazole may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including itraconazole may be applied to skin in powder or solution followed by application of a carrier or second portion of the carrier comprising gel, lotion, ointment, or cream, which may include a second portion of itraconazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including itraconazole may be administered to a body surface to treat or prevent an opportunistic infection comprising Sporothrix schenckii, Trichophyton sp., Trichophyton rubrum, Aspergillus flavus, Aspergillus fumigatus, and/or Candida species, e.g., Candida albicans, Candida auris, Candida glabrata, Candida krusei, and/or Candida tropicalis.

In one embodiment, the method includes combining an active agent comprising one or more antifungals comprising clotrimazole and the carrier. The clotrimazole may be combined with the carrier to formulate emulsions, lotions, creams, ointments, gels, shampoos, nail lacquers, solutions, suspensions, dispersions, irrigations/baths, or pastes, for example. The clotrimazole may comprise commercially available tablets, capsules, vials for injection or solution, bulk power, solutions, topical ointments, creams, lotions, and/or gels, for example. In one embodiment, clotrimazole bulk powder, which may be included in a compounded capsule that may be opened by a user for addition to the carrier, may be used. In one example between about 5 mg and about 50 mg, such as about 20 mg, may be combined with a dosage volume of the carrier. Other strengths may be used.

In various embodiments, an administration dosage may include clotrimazole in an about 20 mg or less, about 20 mg or greater, about 40 mg or greater, about 50 mg or greater, about 75 mg or greater, about 100 mg or greater, or about 150 mg or greater. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include commercially available clotrimazole formats such as tablets, capsules, vials for injection or solution, bulk power, solutions, topical ointments, creams, lotions, and/or gels, for example. The topical composition may be administered to the skin, vagina, nasal passage, or anal cavity.

In one embodiment, the method comprises combining a first portion of the active agent with a second portion of the active agent, wherein the second portion of the active agent comprises a commercially available clotrimazole composition such as a Clotrimazole Cream, Clotrimazole Lotion, Clotrimazole Liquid, or Clotrimazole Solution. The commercially available clotrimazole composition may provide the carrier and/or additional carrier and/or carrier components described herein may be further combined. The first portion of the active agent may include one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof. The topical composition comprising clotrimazole may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including clotrimazole may be applied to skin in powder or solution followed by application of a carrier or second portion of the carrier comprising gel, lotion, ointment, or cream, which may include a second portion of clotrimazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method includes combining an active agent comprising one or more antifungals comprising or consisting of flucytosine and a carrier. The flucytosine may be combined with the carrier to formulate emulsions, lotions, creams, ointments, gels, shampoos, nail lacquers, solutions, suspensions, dispersions, irrigations/baths, nebulizer solutions or suspensions, or pastes, for example. The flucytosine may comprise commercially available tablets, capsules, vials for injection or solution, bulk power, solutions, topical ointments, creams, lotions, and/or gels, for example.

In one example, the active agent comprises flucytosine and a method of formulating the topical composition comprises addition of a flucytosine capsule to a carrier. Less than all the capsule or powder contents thereof may be used when the capsule contains more flucytosine than required for the formulation. More than one capsule may be used when the method includes formulating a topical composition comprising more flucytosine than is in a capsule. The flucytosine capsules may comprise flucytosine 250 mg or 500 mg capsules, USP, for oral administration, for example. Other dose capsules may be used. In some embodiments, in addition to flucytosine and carrier, the topical composition also includes corn starch, lactose and talc; lactose monohydrate, colloidal silicon dioxide, talc, sodium starch glycolate, and magnesium stearate; anhydrous lactose, gelatin, iron oxide black, potassium hydroxide, potato starch, shellac, silicon dioxide, talc and titanium dioxide; glucose polymer and one or more additional components such as magnesium stearate, povidone, lactose, glycol, oxide, talc, or triacetin. In one example, the topical composition may further include croscarmellose sodium, lactose monohydrate, magnesium stearate, povidone and pregelatinized starch. In a further example, the topical composition may include hypromellose, lactose monohydrate, polyethylene glycol, talc and titanium dioxide. In another example, the topical composition includes a starch such as pregelatinized starch, a cellulose such as croscarmellose sodium and/or hypromellose. The topical composition may also include one or more of lactose monohydrate, magnesium stearate, povidone, titanium dioxide, or triacetin. In another example, the topical composition may include a starch, croscarmellose sodium, lactose monohydrate, magnesium stearate, polyethylene glycol, polyvinyl alcohol, povidone, talc, and titanium dioxide.

The method of formulating a topical composition may comprise combining an active agent comprising flucytosine and a carrier. In one example, the flucytosine is combined with the carrier to formulate a topical composition comprising between 0.01% and 50% by weight, such as greater than about 1%, greater than about 3%, greater than about 5%, greater than about 8%, greater than about 10%, greater than about 15%, greater than about 20%, greater than about 25%, between about 0.01% and about 25%, between about 0.5% and about 10%, between about 0.5% and about 5%, between about 0.5% and about 3%, between about 1% and about 20%, between about 1% and about 15%, between about 1% and about 10%, between about 1% and about 5%, between about 1% and about 3%, between about 3% and about 20%, between about 3% and about 15%, between about 3% and about 10%, between about 3% and about 8%, between about 5% and about 25%, between about 5% and about 20%, between about 5% and about 15%, between about 5% and about 10%, between about 8% and about 25%, between about 8% and about 15%, between about 8% and about 10%, between about 10% and about 25%, between about 10% and about 20%, between about 10% and about 15%, between about 15% and about 25%, between about 15% and about 20%, less than about 1%, less than about 3%, less than about 5%, less than about 8%, less than about 10%, less than about 15%, less than about 20%, or less than about 25% flucytosine by weight. In various embodiments, the active agent includes flucytosine and an administration dosage of the topical composition comprises flucytosine in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 1000 mg or greater, greater than about 1250 mg, between about 50 mg and about 1750 mg, between about 50 mg and about 1500 mg, between about 50 mg and about 1000 mg, between about 50 mg and about 200 mg, between about 250 mg and about 500 mg, between about 300 mg and about 800 mg, between about 500 mg and about 1000 mg, between about 250 mg and about 1500 mg, such as between about 250 mg and about 1000 mg, between about 500 mg and about 1750 mg, between about 500 mg and about 1500 mg, such as between about 500 mg and about 1000 mg, between about 250 mg and about 1500 mg, such as between about 750 mg and about 1500 mg, or between about 750 mg and about 1250 mg, between about 250 mg and about 750 mg, between about 400 mg and about 800 mg, between about 200 mg and about 300 mg, or between about 300 mg and about 600 mg. In various embodiments, the topical composition comprises a cream or gel including flucytosine in an amount between about 1% and about 25% by weight, such as between about 5% and about 10%, less than about 5%, less than about 3%, greater than about 10%, such as greater than about 12%, or greater than about 13% by weight. The compounded topical composition may include an additional antibacterial agent, such as those described herein.

In some embodiments, the topical composition comprises flucytosine for topical administration to skin or mucosal tissue. The topical composition may comprise between about 250 mg and about 1.5 g flucytosine. In the above or another embodiment, the topical composition comprises flucytosine and between about 200 mg and about 2 g streptomycin, such as between about 500 mg and about 1.2 g streptomycin. In any of the above or another example, the topical composition comprises flucytosine and between about 25 mg and about 600 mg vancomycin. In any of the above examples or another example, the topical composition comprises flucytosine and keratolytic agent selected from urea, salicylic acid, papain, or combination thereof. In one example, the topical composition comprises between about 250 mg and about 1.5 g flucytosine, between about 500 mg and about 1.2 g streptomycin, and between about 25 mg and about 600 mg vancomycin. In one example, the topical composition comprises between about 500 mg and about 1 g flucytosine, between about 600 mg and about 1 g streptomycin, and between about 100 mg and about 400 mg vancomycin. In one example, the topical composition further comprises corn starch, lactose, and talc; lactose monohydrate, colloidal silicon dioxide, talc, sodium starch glycolate, and magnesium stearate; or anhydrous lactose, potassium hydroxide, and potato starch. In some examples, the topical composition includes flucytosine and a keratolytic agent may be combined in an amount between about 5% and about 50% by weight from urea. In one example, the topical composition comprises between about 300 mg and about 1.2 g urea. In one example, the flucytosine is combined with between about 5% and about 40% urea cream or ointment.

The topical composition may comprises a solution, cream, ointment, gel, paste, or lotion. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. The carrier may be formulated as described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In various embodiments, the topical composition including flucytosine may include one or more additional active agents selected from one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof. For example, additional actives, when included, may include one or more antifungals, antibacterials, keratolytics, antidepressants, local anesthetics, statins, mucolytics, corticosteroids, or combinations thereof in a combined amount between 0.01% and 30% by weight, such as between about 0.01% and about 5% or about 10%. In some embodiments, the amount of flucytosine in the administration dosage is 250 mg or a multiple thereof, such as up to about 1750 mg or about 1500 mg. Additionally or alternatively, additional actives may include other active agents such as one or more active agents selected from an antiviral, NSAID, acid reducer, anticonvulsant or nerve depressant, muscle relaxant, or combination thereof. Such additional actives may be present in a combined amount between 0.01% and 25% by weight, such as between about 1% and about 10%. Bulk powder or powder contents of one or more capsules or tablets comprising the active may be added to a volume of the carrier sufficient to formulate an administration dosage of the topical composition having the desired strength of the flucytosine along with the one or more additional actives. For example, the topical composition may comprise an active agent comprising or consisting of an antifungal. The antifungal may comprise or consist of flucytosine as described herein and may be formulated for topical administration to skin, nails, hair, oral cavity, nasal cavity, or respiratory tract, such as lower respiratory tract or lungs. In one example, the topical composition comprises flucytosine together with another antifungal, such as an azole described herein, e.g., itraconazole. In another example, flucytosine is provided without another antifungal or without an azole. The active agent may further comprise or consist of between about 50 mg to about 200 mg amphotericin, between about 50 mg to about 600 mg econazole, between about 50 mg to about 200 mg fluconazole, between about 125 mg and about 500 mg griseofulvin, between about 50 mg to about 200 mg ketoconazole, between about 100 mg to about 200 mg metronidazole, between about 100 mg to about 550 mg nystatin, between about 25 mg to about 550 mg itraconazole, between about 20 mg to about 150 mg clotrimazole, or between about 50 mg to about 600 mg voriconazole. In an above or another example, the active agent including flucytosine may further comprise or consist of an antibacterial described herein in an amount between about 0.01% and about 50% by weight of the topical composition, such as between about 400 mg and about 2.2 g amoxicillin, between about 400 mg and about 2 g ampicillin, between about 100 mg and about 1.5 g azithromycin, between about 100 mg and about 1.5 g cefaclor, between about 400 mg and about 2.0 g cefadroxil, between about 100 mg and about 500 mg cefdinir, between about 400 mg and about 2.0 g cefazolin, between 400 mg and about 2.5 g cefepime, between about 250 mg and about 2.5 g cephalexin, between about 100 mg and about 1.5 g cefixime, between about 50 mg and about 600 mg cefpodoxime, between about 400 mg and about 2 g cefotetan, between about 100 mg and about 1.5 g cefprozil, between about 400 mg and about 3 g ceftriaxone, between about 100 mg and about 1.5 g cefuroxime, between about 400 mg and about 2.5 g ceftazidime, between about 100 mg and about 1.5 g ciprofloxacin, between about 100 mg and about 1.5 g clarithromycin, between about 75 mg and about 1 g clindamycin, between about 50 mg and about 600 mg colistimethate, between about 100 mg and about 250 mg doxycycline, between about 100 mg and about 1.5 mg erythromycin, between about 100 mg and about 600 mg gentamicin, between about 100 mg and about 1 g isoniazid, between about 100 mg and about 1 g levofloxacin, between about 100 mg and about 1.8 g linezolid, between about 50 mg and about 1.5 mg meropenem, between about 100 mg and about 1.5 g moxifloxacin, between about 20 mg and about 1 g mupirocin, between about 200 mg and about 5 g nafcillin, between about 10 mg and about 600 mg nitrofurantoin, between about 100 mg and about 1.5 g ofloxacin, between about 100 mg and about 1.5 g tetracycline, between about 20 mg and about 2.5 g tobramycin, between about 200 mg and about 2.0 g streptomycin, between about 80 mg and about 160 mg sulfamethoxazole and between about 50 mg and about 100 mg trimethoprim, between about 25 mg and about 600 mg vancomycin, or combination thereof. In an above or another example, the active agent including flucytosine may further comprise or consist of one or more antivirals described herein in an amount between about 0.01%, about 50% between about 0.01% and about 20% by weight, or between about 0.5% and about 5% by weight such as acyclovir in an amount between about 100 mg and about 1.8, famciclovir in an amount between about 100 mg and about 1.5 g, valaciclovir in an amount between about 200 mg and about 2 g, or combination thereof. In an above or another example, the active agent including flucytosine may further comprise or consist of one or more NSAIDs described herein in an amount between about 0.01% and about 40% or between about 1% and about 20% by weight, such as celecoxib in an amount between about 50 mg and about 1.5 g, etodolac in an amount between about 50 mg and about 700 mg, indomethacin in an amount between about 25 mg and about 200 mg, diclofenac in an amount between about 5 mg and about 1 g, nabumetone in an amount between about 200 mg and about 2 g, or combination thereof. In an above or another example, the active agent including flucytosine may further comprise or consist of one or more statins described herein in an amount between about 0.01% and about 50%, between about 5% and about 25%, or between about 0.01% and about 15% by weight, such as between about 0.5% and about 8%, or in an amount between about 10 mg and about 1.5 g or as otherwise described herein. In an above or another example, the active agent including flucytosine may further comprise or consist of an antidepressants in an amount described herein such as between about 0.0001% and about 50% or between about 0.01% and about 15% by weight, or in an amount between about 10 mg and about 1 g. In an above or another example, the active agent including flucytosine may further comprise or consist of one or more mucolytics described herein in an amount between about 0.01% and about 50%, between about 5% and about 25%, or between about 0.01% and about 15% by weight, such as between about 0.5% and about 8%, or in an amount between about 10 mg and about 1.5 g or as otherwise described herein.

In one example, the topical composition including flucytosine is formulated for nasal administration, either to nasal mucosa, nebulization, and/or intranasally to the lungs and includes sodium citrate. Administration to the lower respiratory tract may therapeutically treat respiratory tract infections, e.g., Candida albicans, Cryptococcus, Pseudomonas aeruginosa, or other infections, such as those identified herein. In various embodiments, the topical composition including flucytosine described herein may be administered to a subject to treat fungal and/or bacterial infections, with or without further incorporation of antibacterials.

In various embodiments, a method of formulating the topical composition comprises combining flucytosine in an amount identified herein in combination with one or more additional actives such as antibacterials, antivirals, local anesthetics, antidepressants, steroids, NSAIDs, statins, keratolytics, acid reducers, calcium channel blockers, antianxiety drugs, mucolytics, or antihistamines, including combinations thereof, in an amount between about 0.001% and about 50% by weight, which is to be understood to include any weight or range of weights therebetween, such as any weight or range or weight or percent composition disclosed herein. In an above or another embodiment, the method comprises combining one or more identified antifungals in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight, such as any weight or weight range or percent composition disclosed herein. The one or more additional actives may comprise tablets, capsules, vials for injection or solution, bulk power, solutions, topical ointments, creams, lotions, and/or gels, for example.

The topical composition comprising flucytosine may be administered by contacting to a body surface, e.g., skin, mucosal tissue. The composition may be administered to an infected or target area via spray, drops, wash, mouthwash/rinse, swab, sponge, absorbent dressing, coating (e.g., a nail lacquer), soaking, submerging, footbath, instillation or irrigation. Carries or diluents may be as described herein or otherwise as suitably known in the art. Administration may be directed to the upper or lower respiratory tract, such as the lungs, via the mouth or intranasally by inhalation or nebulization of the topical composition in a solution, suspension, emulsion, or powder format. Nebulization may be by larger or small particle nebulization. In one embodiment, the topical composition may also include sodium citrate and comprise a powder or solution for administration to the nasal cavity by inhalation, irrigation, spray, drop, or large particle nebulization. In various embodiments, the composition may be administered in a combinational treatment in a manner described above or elsewhere herein. For example, the composition including flucytosine alone or together with one or more additional actives may be applied to skin in powder or solution followed by application of a carrier or second portion of the carrier comprising gel, lotion, ointment, or cream, which may include a second portion of flucytosine and/or another active or not include a second portion of the active agent. In one embodiment, an anhydrous ointment is applied over the powder or a water washable ointment, such as Bassa-gel, is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including flucytosine alone or together with another active agent may be administered to a body surface to treat or prevent an opportunistic infection comprising Acinetobacter, MRA, MSSA, Klebsiella species such as Klebsiella pneumoniae, Klebsiella oxytoca, Cryptococcus neoformans, Trichophyton interdigitale, Trichophyton rubrum, Scedosporium apiospermum, Sporothrix schenckii, Aspergillus fumigatus, and/or Candida species, e.g., Candida kefyr, Candida lusitaniae, Candida parapsilosis, Candida pararugosa, Candida albicans, Candida pelliculosa, Candida glabrata, and/or Candida krusei. The topical composition may be used as a treatment for arthritis or joint diseases associated with ankylosing spondylitis and other spondyloarthropathies. The topical composition may be utilized in a treatment of a Proteus infection. The topical composition may be administered vaginally, anally, to skin, ear/otic, nasal cavity, or lungs. The topical composition may be administered to a subject to treat a bacterial infection, such as those described above or elsewhere herein, wherein flucytosine provides all or a portion of the antibacterial action. In one example, the topical composition including flucytosine is administered to treat a bacterial infection and does not include a currently recognized pharmaceutical antibiotic. In some examples, a topical composition described herein may include flucytosine substituted for an aminoglycoside, glycosamide, macrolide, or quinoline. In one example, a topical composition comprising nitrofurantoin or clindamycin may instead include flucytosine substituted for the nitrofurantoin or clindamycin.

In one example, the topical composition may include a regime of multiple administrations of a combination of actives. The topical composition may comprise gentamycin in an amount between about 50 mg and about 200 mg, clindamycin in an amount between about 50 mg and about 150 mg, mupirocin in an amount between about 10 mg and about 40 mg, methylprednisolone in an amount between about 2 mg and about 8 mg, and flucytosine in an amount between about 250 mg and about 1 g. The above combination may be administered once or twice a day. In one example, the topical composition is configured for nasal administration. A first composition comprising about 60 mg to 120 mg gentamycin, about 80 mg to about 130 mg clindamycin, and about 10 mg to about 40 mg mupirocin may be combined with distilled water and nasally administered. A second composition comprising about 70 mg to about 130 mg gentamycin, and about 2 mg to about 8 mg methylprednisolone may be combined with distilled water and nasally administered. A third composition comprising between about 250 mg and about 1 g flucytosine may be combined with distilled water and nasally administered. The three compositions each be administered once or twice daily. In one example the nasal administration comprises nasal irrigation. In the above or another example, the first composition comprises about 80 mg gentamycin, about 100 mg clindamycin, about 20 mg mupirocin, the second composition comprises about 90 mg gentamycin and about 5 mg methylprednisolone, and the third composition comprises about 500 mg flucytosine. In some embodiments, the combination of actives may be administered in two compositions or a single composition. Other combinations may be used. In some embodiments, the above combinations of actives may be administered as a powder to skin or nasal mucosal tissue.

In one embodiment, the method includes combining an active agent comprising one or more antifungals comprising voriconazole and the carrier. The voriconazole may be combined with the carrier to formulate emulsions, lotions, creams, ointments, gels, shampoos, nail lacquers, solutions, suspensions, dispersions, irrigations/baths, or pastes, for example. The voriconazole may comprise commercially available tablets, capsules, vials for injection or solution, bulk power, solutions, topical ointments, creams, lotions, and/or gels, for example.

In one example, the active agent comprises voriconazole and a method of formulating the topical composition comprises addition of a crushed voriconazole tablet to a carrier. Less than all the powder of a crushed tablet may be used when the tablet contains more voriconazole than required. More than one crushed tablet may be used when the method includes formulating a topical composition comprising more voriconazole than is in the tablet. The voriconazole tablets may comprise commercially available voriconazole 50 mg, 100 mg, 200 mg oral tablets. In some embodiments, other strength tablets may be used. In addition to voriconazole the powder may include a glucose polymer and one or more additional components such as magnesium stearate, povidone, lactose, glycol, oxide, talc, triacetin, or an alcohol. In one example, the powder includes croscarmellose sodium, lactose monohydrate, magnesium stearate, povidone and pregelatinized starch. In a further example, the powder may include hypromellose, lactose monohydrate, polyethylene glycol, talc and titanium dioxide. In another example, the powder may include a starch such as pregelatinized starch, a cellulose such as croscarmellose sodium and/or hypromellose. The powder may also include one or more of lactose monohydrate, magnesium stearate, povidone, titanium dioxide, or triacetin. In another example, the powder may include a starch, croscarmellose sodium, lactose monohydrate, magnesium stearate, polyethylene glycol, polyvinyl alcohol, povidone, talc, and titanium dioxide. In another example, the powder may further include talc. In one example, the powder includes lactose monohydrate, pregelatinized starch (corn), croscarmellose sodium, povidone, magnesium stearate and a coating containing polyvinyl alcohol-part hydrolyzed, titanium dioxide, macrogol/PEG and talc. In one embodiment, the powder may include pregelatinized starch, croscarmellose sodium, lactose monohydrate, magnesium stearate, povidone, and a coating containing hypromellose, lactose monohydrate, titanium dioxide and triacetin.

The method of formulating a topical composition may comprise combining an active agent comprising crushed oral tablets and a carrier. In one example, the active agent comprises an antifungal comprising voriconazole and the method of formulating the topical composition comprises addition of a crushed voriconazole tablet to the carrier. The voriconazole tablets may comprise commercially available voriconazole 50 mg, 100 mg, 200 mg oral tablets. The oral tablets may be crushed and combined with the carrier to formulate a topical composition comprising between 0.01% and 20% by weight, such as about 1%, about 2%, about 3%, about 4%, about 5%, less than about 5%, between about 2% and about 7%, or greater than about 10% voriconazole by weight. To formulate a topical composition comprising a desired percent by weight voriconazole, the total desired weight of the topical composition is subtracted from the weight of crushed oral voriconazole tablet powder needed to obtain the desired percent by weight voriconazole. The weight of voriconazole tablet powder needed is determined by multiplying the weight of active needed to obtain the desired percent by weight voriconazole in the topical composition. For example, a topical composition comprising 1% voriconazole may be formulated combining powder obtained from 200 mg oral voriconazole tablets. The weight of voriconazole tablet powder needed is determined by multiplying the weight of voriconazole needed to obtain the desired percent by weight voriconazole in the topical composition. Here, a 1% voriconazole composition includes 10 mg voriconazole per gram. If a 200 mg voriconazole tablet weights about 450 mg, 22.5 mg of crushed voriconazole tablet powder comprises 10 mg voriconazole. Therefore, 22.5 mg of crushed voriconazole tablet powder is combined for each gram of topical composition. Consequently, 977.5 mg of carrier, carrier components thereof, and additional active agents, if any, may be combined with 22.5 mg of crushed voriconazole tablet powder to formulate each gram of topical composition to formulate a 1% by weight topical composition. Other percent compositions may be formulated as described herein. Additional actives may include one or more antifungals, antibacterials, keratolytics, antidepressants, local anesthetics, or combinations thereof in a combined amount between 0.01% and 20% by weight, such as between about 0.01% and about 5%. Additionally or alternatively, additional actives may include other active agents such as one or more active agents selected from an antiviral, NSAID, anticonvulsant or nerve depressant, muscle relaxant, or combination thereof. Such additional actives may be present in a combined amount between 0.01% and 25% by weight, such as between about 1% and about 10%. The one or more tablets may be added to a volume of the carrier sufficient to formulate an administration dosage of the topical composition having the desired strength of the voriconazole. In some embodiments, the amount of voriconazole in the administration dosage corresponds to the amount of voriconazole present in one tablet, or a multiple thereof. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application.

In various embodiments, the active agent includes voriconazole and an administration dosage of the topical composition comprises voriconazole in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available voriconazole formats such as capsules, vials for injection or solution, bulk power, solutions, topical ointments, creams, lotions, and/or gels, for example. For example, all or a portion of a 200 mg vial of Voriconazole for Injection containing about 200 mg voriconazole and 3200 mg sulfobutyl ether beta-cyclodextrin sodium may be used. The topical composition may be administered to the skin, vagina, nasal passage, or anal cavity. The topical composition comprising voriconazole may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including voriconazole may be applied to skin in powder or solution followed by application of a carrier or second portion of the carrier comprising gel, lotion, ointment, or cream, which may include a second portion of voriconazole and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including voriconazole may be administered to a body surface to treat or prevent an opportunistic infection comprising Cryptococcus neoformans, Trichophyton interdigitale, Trichophyton rubrum, Scedosporium apiospermum, Sporothrix schenckii, Aspergillus fumigatus, and/or Candida species, e.g., Candida kefyr, Candida lusitaniae, Candida parapsilosis, Candida pararugosa, Candida pelliculosa, Candida glabrata, and/or Candida krusei.

In one embodiment, the method comprises combining a first portion of the active agent with a second portion of the active agent, wherein the second portion of the active agent comprises a commercially available voriconazole composition such as Voriconazole Ophthalmic Ointment or Voriconazole Oral Suspension. The Voriconazole Oral Suspension may include 45 g powder for oral suspension for reconstitution with water to produce a suspension containing 40 mg/mL voriconazole and including colloidal silicon dioxide, titanium dioxide, xanthan gum, sodium citrate dihydrate, sodium benzoate, anhydrous citric acid, natural orange flavor, and sucrose. The commercially available voriconazole composition may provide the carrier and/or additional carrier and/or carrier components described herein may be further combined. The first portion of the active agent may include one or more herein described antifungals, antibacterials, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof.

In various embodiments, the method comprises combining one or more antifungals identified herein in combination with one or more antibacterials, antivirals, local anesthetics, antidepressants, steroids, NSAIDs, statins, keratolytics, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified antifungals in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one example, the method includes combining one or more antifungals, one or more antibacterials, and the carrier. The antifungal and antibacterial drugs may comprise any combination of tablets, capsules, vials for injection or solution, bulk power, solutions, topical ointments, creams, lotions, and/or gels, for example. In one example, the combination actives may be provided in one or more compounded capsules including bulk powder wherein the compounded capsule may be opened and its contents combined with the carrier. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel.

In one embodiment, the method includes combining clindamycin and flucytosine with a carrier. Clindamycin and flucytosine may be combined at a weight ratio between about 1:2 to about 4:1. In one formulation, the method comprises adding about 50 mg flucytosine for about every 100 mg clindamycin. In one embodiment, an administration volume of the topical composition comprises about 50 mg flucytosine and about 100 mg clindamycin. The clindamycin and flucytosine may be provided in one or more compounded capsules including bulk powder wherein the compounded capsule may be opened and its contents combined with the carrier. As noted above, some embodiments may include other commercially available formats of clindamycin and/or flucytosine such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining doxycycline and flucytosine. Doxycycline and flucytosine may be combined at a weight ratio between about 1:2 to about 4:1. In one formulation, the method comprises adding 50 mg flucytosine for every 100 mg doxycycline. In one embodiment, an administration volume of the topical composition comprises about 50 mg flucytosine and about 100 mg doxycycline. The doxycycline and flucytosine may be provided in one or more compounded capsules including bulk powder wherein the compounded capsule may be opened and its contents combined with the carrier. As noted above, some embodiments may include other commercially available formats of doxycycline and/or flucytosine such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining mupirocin and flucytosine. Mupirocin and flucytosine may be combined at a weight ratio between about 1:5 to about 2:1. In one formulation, the method comprises adding 50 mg flucytosine for every 20 mg mupirocin. In one embodiment, an administration volume of the topical composition comprises about 50 mg flucytosine and about 20 mg mupirocin. The mupirocin and flucytosine may be provided in one or more compounded capsules including bulk powder wherein the compounded capsule may be opened and its contents combined with the carrier. As noted above, some embodiments may include other commercially available formats of mupirocin and/or flucytosine such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining mupirocin, clindamycin, and flucytosine. The clindamycin and flucytosine may be combined at a weight ratio between about 1:2 to about 4:1, the mupirocin and clindamycin may be combined at a weight ratio of about 1:2 to about 4:1, and the mupirocin and flucytosine may be combined at a weight ratio of about 1:2 to about 4:1. In one embodiment, the method comprises adding about 20 mg mupirocin for about every 25 mg of each of clindamycin and flucytosine. In one embodiment, an administration volume of the topical composition comprises about 20 mg mupirocin and about 25 mg of each of clindamycin and flucytosine. The mupirocin, clindamycin, and flucytosine may be provided in one or more compounded capsules including bulk powder wherein the compounded capsule may be opened and its contents combined with the carrier. As noted above, some embodiments may include other commercially available formats of mupirocin, clindamycin, and/or flucytosine such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining levofloxacin, clindamycin, and flucytosine. The clindamycin and flucytosine may be combined at a weight ratio between about 1:2 to about 4:1, the levofloxacin and clindamycin may be combined at a weight ratio of about 1:2 to about 4:1, and the levofloxacin and flucytosine may be combined at a weight ratio of about 1:2 to about 4:1. In one embodiment, the method comprises adding about 25 mg clindamycin for about every 50 mg of each of levofloxacin and flucytosine. In one embodiment, an administration volume of the topical composition comprises about 25 mg clindamycin and about 50 mg of each of levofloxacin and flucytosine. The levofloxacin, clindamycin, and flucytosine may be provided in one or more compounded capsules including bulk powder wherein the compounded capsule may be opened and its contents combined with the carrier. As noted above, some embodiments may include other commercially available formats of levofloxacin, clindamycin, and/or flucytosine such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining tobramycin, mupirocin, clindamycin, and flucytosine. The clindamycin and flucytosine may be combined at a weight ratio between about 1:2 to about 4:1, the mupirocin and clindamycin may be combined at a weight ratio of about 1:2 to about 4:1, the mupirocin and flucytosine may be combined at a weight ratio of about 1:2 to about 4:1, the tobramycin and flucytosine may be combined at a weight ratio between about 1:2 to about 8:1, the tobramycin and clindamycin may be combined at a weight ratio of about 1:2 to about 8:1, the tobramycin and mupirocin may be combined at a weight ratio of about 1:2 to about 8:1. In one embodiment, the method comprises adding about 100 mg tobramycin for about every 25 mg of each of mupirocin, clindamycin and flucytosine. In one embodiment, an administration volume of the topical composition comprises about 100 mg tobramycin and about 25 mg of each of mupirocin, clindamycin, and flucytosine. The tobramycin, mupirocin, and clindamycin may be provided in one or more compounded capsules including bulk powder wherein the compounded capsule may be opened and its contents combined with the carrier. As noted above, some embodiments may include other commercially available formats of tobramycin, mupirocin, and/or clindamycin such as gels, for example. The topical composition may be administered to the skin, vagina, nasal passage, or anal cavity.

In an embodiment, the method of formulating the topical composition may comprise combining with a carrier an active agent comprising one or more antibacterial drugs, such as any of the antibacterials described herein in an amount between about 0.01% and about 50% by weight of the topical composition. The active agent may be combined with the carrier to formulate creams, ointments, solutions/irrigations/baths, powders, gels, lotions, or pastes, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The antibacterials drugs may comprise tablets, capsules, vials for injection or solution, bulk power, solutions, topical ointments, creams, lotions, and/or gels, for example. In one embodiment, the method of making the topical composition comprises combining with the carrier one or more antibacterials selected from enicillins, cephalosporins, fluoroquinolones, aminoglycosides, monobactams, carbapenems, macrolides, or combination thereof. For example, a method may include combining the carrier with one or more of afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In one example, the antibacterial comprises amoxicillin, ampicillin, azithromycin, cefaclor, cefadroxil, cefazolin, cefepime, cefixime, cefpodoxime, cefprozil, ceftriaxone, cefuroxime, ceftazidime, ciprofloxacin, clarithromycin, clindamycin, colistimethate, doxycycline, erythromycin, gentamicin, isoniazid, levofloxacin, linezolid, ofloxacin, nafcillin, nitrofurantoin, mupirocin, tobramycin, vancomycin, and combinations thereof. The method of formulating the topical composition may also include combining on or more additional portions of the active agent selected from one or more herein identified antibacterials, antifungals, antivirals, NSAIDs, keratolytics, statin, antidepressants, anticonvulsants, steroids, local anesthetics, acid reducers, calcium channel blockers, antianxiety drugs, mucolytics, antihistamines, stimulants, disinfectants, nerve depressants, muscle relaxants, NMDA (N-Methyl-D-aspartate) receptor antagonists, opiate or opioid agonists, anticholinergics and/or other active agents. The topical composition may be administered to the skin, vagina, nasal.

In one embodiment, the method includes combining an active agent comprising one or more antibacterials comprising amoxicillin and the carrier. The amoxicillin may be combined with the carrier to formulate emulsions, lotions, creams, ointments, gels, shampoos, nail lacquers, solutions, suspensions, dispersions, irrigations/baths, or pastes, for example. The amoxicillin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining one or more 500 mg and/or 875 mg amoxicillin oral tablets with a carrier. Each amoxicillin tablet may contain about 500 mg or 875 mg contains amoxicillin USP as the trihydrate. The tablets may also include one or more of colloidal silicon dioxide, crospovidone, ethylcellulose aqueous dispersion, hypromellose, magnesium stearate, microcrystalline cellulose, sodium starch glycolate, talc, triethyl citrate, and/or titanium dioxide. In one example, each tablet contains 500 mg or 875 mg amoxicillin USP, colloidal silicone dioxide, crospovidone, magnesium stearate, microcrystalline cellulose, polyvinyl alcohol-partially hydrolyzed, polyethylene glycol, sodium starch glycolate, talc, and titanium dioxide. In one embodiment, the amoxicillin tablets include amoxicillin and clavulanate potassium tablets comprising amoxicillin, clavulanate potassium and one or more of colloidal silicon dioxide, ethylcellulose, hypromellose, magnesium stearate, microcrystalline cellulose, propylene glycol, sodium starch glycolate, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the amoxicillin. In some embodiments, the amount of amoxicillin in the administration dosage corresponds to the amount of amoxicillin present in one tablet, or a multiple thereof.

In various embodiments, the active agent includes amoxicillin and an administration dosage of the topical composition comprises amoxicillin in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 1.4 g or greater, between about 400 mg and about 700 mg, between about 700 mg and about 1 g, between about 1 g and about 1.3 g, between about 1.3 g and about 1.6 g, between about 1.6 g and about 1.9 g, or between about 1.9 g and about 2.4 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available amoxicillin formats such as capsules, vials for injection or solution, bulk power, solutions, topical ointments, creams, lotions, and/or gels, for example. The topical composition may be administered to the skin, vagina, nasal passage, or anal cavity. The topical composition comprising amoxicillin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including amoxicillin may be applied to skin in powder or solution followed by application of a carrier or second portion of the carrier comprising gel, lotion, ointment, or cream, which may include a second portion of amoxicillin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method includes combining an active agent comprising one or more antibacterials comprising ampicillin and the carrier. The ampicillin may be combined with the carrier to formulate emulsions, lotions, creams, ointments, gels, shampoos, nail lacquers, solutions, suspensions, dispersions, irrigations/baths, or pastes, for example. The ampicillin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining one or more 250 mg and/or 500 mg ampicillin oral capsules and/or the contents thereof with a carrier. Each ampicillin oral capsule includes ampicillin trihydrate equivalent to about 250 mg or about 500 mg ampicillin and one or more of magnesium stearate, titanium dioxide, propylene glycol, ammonium hydroxide, and/or potassium hydroxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the ampicillin. In some embodiments, the amount of ampicillin in the administration dosage corresponds to the amount of ampicillin present in one capsule, or a multiple thereof. In one example, the method includes combining one or more vials of ampicillin sodium injection, powder, for solution. Each vial contains ampicillin sodium equivalent to about 250 mg, about 500 mg, about 1 g, and/or about 2 g ampicillin and about 66 mg sodium per gram of ampicillin. Other strengths may be used. The contents of the one or more vials may be added to a volume of the carrier sufficient to formulate an administration dosage of the topical composition having the desired strength of the ampicillin. In some embodiments, the amount of ampicillin in the administration dosage corresponds to the amount of ampicillin present in a vial, or a multiple thereof.

In various embodiments, the active agent includes ampicillin and an administration dosage of the topical composition comprises ampicillin in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, about 4.5 g or greater, about 5 g or greater, about 5.5 g or greater, about 6 g or greater, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available ampicillin formats such as capsules, vials for injection or solution, bulk power, solutions, topical ointments, creams, lotions, and/or gels, for example. The topical composition may be administered to the skin, vagina, nasal passage, or anal cavity. The topical composition comprising ampicillin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including ampicillin may be applied to skin in powder or solution followed by application of a carrier or second portion of the carrier comprising gel, lotion, ointment, or cream, which may include a second portion of ampicillin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method includes combining an active agent comprising one or more antibacterials comprising azithromycin and the carrier. The azithromycin may be combined with the carrier to formulate emulsions, lotions, creams, ointments, gels, shampoos, nail lacquers, solutions, suspensions, dispersions, irrigations/baths, or pastes, for example. The azithromycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, at least a portion of the azithromycin may comprise a commercially available azithromycin, such as Azithromycin for Injection USP, which may be supplied in lyophilized form under a vacuum in a 10 mL vial equivalent to 500 mg of azithromycin for intravenous administration including sodium hydroxide and 413.6 mg citric acid; Azithromycin for Oral Suspension, USP, which may be supplied for suspension in 100 mg/5 mL or 200 mg/5 mL; Azithromycin Tablets; or bulk powder. In one formulation, the azithromycin comprises a commercially available azithromycin such as 250 mg and/or 500 mg azithromycin oral tablets and a method of formulating the topical composition comprises addition of a crushed azithromycin tablet to the carrier. Less than all the powder of a crushed tablet may be used when the tablet contains more azithromycin than required. More than one crushed tablet may be used when the method includes formulating a topical composition comprising more azithromycin than is in the tablet. The azithromycin tablets may comprise commercially available azithromycin 250 mg and/or 500 mg oral tablets. In some embodiments, other strength tablets may be used. Each azithromycin tablet may contain azithromycin monohydrate equivalent to either 250 mg or 500 mg of azithromycin and one or more of colloidal silicon dioxide, magnesium stearate, microcrystalline cellulose, starch, sodium lauryl sulfate, and/or titanium dioxide. Tablets may also contain one or more of lecithin, polyvinyl alcohol, sodium starch glycolate, talc, xanthan gum, dibasic calcium phosphate anhydrous, croscarmellose sodium, hypromellose, lactose monohydrate, sodium citrate, magnesium trisilicate, and/or hydroxypropyl cellulose. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the azithromycin. In some embodiments, the amount of azithromycin in the administration dosage corresponds to the amount of azithromycin present in one tablet or vial, or a multiple thereof.

In various embodiments, the active agent includes azithromycin and an administration dosage of the topical composition comprises azithromycin in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available azithromycin formats such as capsules, vials for injection or solution, bulk power, solutions, topical ointments, creams, lotions, and/or gels, for example. The topical composition may be administered to the skin, vagina, nasal passage, or anal cavity. The topical composition comprising azithromycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including azithromycin may be applied to skin in powder or solution followed by application of a carrier or second portion of the carrier comprising gel, lotion, ointment, or cream, which may include a second portion of azithromycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including azithromycin may be administered to a body surface to treat or prevent an opportunistic infection comprising Clostridium perfringens, Chlamydia pneumoniae, Chlamydia psittaci, Chlamydia trachomatis, Mycoplasma pneumoniae, Bordetella pertussis, Campylobacter jejuni, Haemophilus ducreyi, Haemophilus influenzae, Klebsiella (Calymmatobacterium) granulomatis, Legionella pneumophila, Moraxella catarrhalis, Neisseria gonorrhoeae, Neisseria meningitidis, Rickettsiae, Salmonella typhi, Shigella sp., Vibrio cholerae, Peptostreptococcus, Staphylococcus aureus (MSSA), Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising cefaclor and the carrier. The cefaclor may be combined with the carrier to formulate emulsions, lotions, creams, ointments, gels, shampoos, nail lacquers, solutions, suspensions, dispersions, irrigations/baths, or pastes, for example. The cefaclor may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the cefaclor comprises a commercially available cefaclor such as one or more cefaclor capsules and/or the contents thereof comprising cefaclor monohydrate equivalent to about 250 mg or about 500 mg of anhydrous cefaclor and one or more of magnesium stearate, sodium starch glycolate, lactose monohydrate, and/or talc. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the cefaclor. In some embodiments, the amount of cefaclor in the administration dosage corresponds to the amount of cefaclor present in one capsule, or a multiple thereof.

In various embodiments, the active agent includes cefaclor and an administration dosage of the topical composition comprises cefaclor in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefaclor formats such as vials for injection or solution, bulk power, solutions, topical ointments, creams, lotions, and/or gels, for example. The topical composition may be administered to the skin, vagina, nasal passage, or anal cavity. The topical composition comprising cefaclor may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefaclor may be applied to skin in powder or solution followed by application of a carrier or second portion of the carrier comprising gel, lotion, ointment, or cream, which may include a second portion of cefaclor and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cefadroxil and the carrier. The cefadroxil may be combined with the carrier to formulate emulsions, lotions, creams, ointments, gels, shampoos, nail lacquers, solutions, suspensions, dispersions, irrigations/baths, or pastes, for example. The cefadroxil may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the cefadroxil comprises a commercially available cefadroxil such as one or more cefadroxil capsules and/or the contents thereof comprising about 500 mg cefadroxil and one or more of colloidal silicon dioxide, croscarmellose sodium, gelatin, lactose monohydrate, magnesium stearate, microcrystalline cellulose, potassium hydroxide, propylene glycol, lactose monohydrate, magnesium stearate, sodium lauryl sulphate, and/or titanium dioxide. Other strengths may be used. The one or more capsules may be preferably opened to release the contents for combining the contents with the carrier. In one example, the cefadroxil comprises a commercially available cefadroxil oral tablet comprising about 1000 mg cefadroxil and one or more of colloidal silicon dioxide, croscarmellose sodium, magnesium stearate, and/or microcrystalline cellulose. Other strengths may be used. The one or more tablets may be crushed or otherwise ground to a powder, typically prior to combining with the carrier for ease of mixing. The one or more tablets and/or capsules, which may be the contents thereof, may be added to a volume of the carrier sufficient to formulate an administration dosage of the topical composition having the desired strength of the cefadroxil. In some embodiments, the amount of cefadroxil in the administration dosage corresponds to the amount of cefadroxil present in one tablet or tablet, or a multiple thereof.

In various embodiments, the active agent includes cefadroxil and an administration dosage of the topical composition comprises cefadroxil in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.5 g or greater, about 1.4 g or greater, between about 400 mg and about 700 mg, between about 700 mg and about 1 g, between about 1 g and about 1.3 g, between about 1.3 g and about 1.6 g, between about 1.6 g and about 2.0 g, or between about 2.0 g and about 3.0 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefadroxil formats such as vials for injection or solution, bulk power, solutions, topical ointments, creams, lotions, and/or gels, for example. The topical composition may be administered to the skin, vagina, nasal passage, or anal cavity. The topical composition comprising cefadroxil may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefadroxil may be applied to skin in powder or solution followed by application of a carrier or second portion of the carrier comprising gel, lotion, ointment, or cream, which may include a second portion of cefadroxil and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cefazolin and the carrier. The cefazolin may be combined with the carrier to formulate emulsions, lotions, creams, ointments, gels, shampoos, nail lacquers, solutions, suspensions, dispersions, irrigations/baths, or pastes, for example. The cefazolin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the cefazolin comprises one of more vials of Cefazolin for Injection, powder, USP comprising cefazolin sodium equivalent to about 10 mg, about 500 mg, or about 1 g cefazolin. Other strengths may be used. The contents of the one or more vials may be added to a volume of the carrier sufficient to formulate an administration dosage of the topical composition having the desired strength of the cefazolin. In some embodiments, the amount of cefazolin in the administration dosage corresponds to the amount of cefazolin present in a vial, or a multiple thereof.

In various embodiments, the active agent includes cefazolin and an administration dosage of the topical composition comprises cefazolin in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.5 g or greater, about 1.4 g or greater, between about 400 mg and about 700 mg, between about 700 mg and about 1 g, between about 1 g and about 1.3 g, between about 1.3 g and about 1.6 g, between about 1.6 g and about 2.0 g, or between about 2.0 g and about 3.0 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefazolin formats such as capsules, capsules, bulk power, solutions, topical ointments, creams, lotions, and/or gels, for example. The topical composition may be administered to the skin, vagina, nasal passage, or anal cavity. The topical composition comprising cefazolin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefazolin may be applied to skin in powder or solution followed by application of a carrier or second portion of the carrier comprising gel, lotion, ointment, or cream, which may include a second portion of cefazolin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including cefazolin may be administered to a body surface to treat or prevent an opportunistic infection comprising Clostridium perfringens, Escherichia coli, Haemophilus influenzae, Klebsiella (Calymmatobacterium) granulomatis, Klebsiella oxytoca, Klebsiella pneumoniae, Moraxella catarrhalis, Neisseria gonorrhoeae, Proteus mirabilis, Proteus vulgaris, Staphylococcus aureus (MSSA), Staphylococcus epidermidis, Streptococcus agalactiae(B), Streptococcus pneumoniae, Streptococcus pyogenes(A), and/or Viridans group streptococci.

In one embodiment, the method includes combining an active agent comprising one or more antibacterials comprising cefdinir and the carrier. The cefdinir may be combined with the carrier to formulate emulsions, lotions, creams, ointments, gels, shampoos, nail lacquers, solutions, suspensions, dispersions, irrigations/baths, or pastes, for example. The cefdinir may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the cefdinir comprises a commercially available cefdinir such as one or more cefdinir capsules and/or the contents thereof comprising about 300 mg cefdinir and one or more of carboxymethylcellulose calcium, colloidal silicon dioxide, and/or magnesium stearate. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the cefdinir. In some embodiments, the amount of cefdinir in the administration dosage corresponds to the amount of cefdinir present in one capsule, or a multiple thereof.

In various embodiments, the active agent includes cefdinir and an administration dosage of the topical composition comprises cefdinir in an amount about 100 mg or less, about 100 mg or greater, about 150 mg or greater, about 200 mg or greater, about 250 mg or greater, about 350 mg or greater, about 450 mg or greater, about 550 mg or greater, about 650 mg or greater, about 850 mg or greater, about 1 g or greater, between about 100 mg and about 200 mg, between about 200 mg and about 300 mg, between about 300 mg and about 500 mg, or between about 500 mg and about 1 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefdinir formats such as vials for injection or solution, bulk power, solutions, topical ointments, creams, lotions, and/or gels, for example. The topical composition may be administered to the skin, vagina, nasal passage, or anal cavity. The topical composition comprising cefdinir may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefdinir may be applied to skin in powder or solution followed by application of a carrier or second portion of the carrier comprising gel, lotion, ointment, or cream, which may include a second portion of cefdinir and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cefepime and the carrier. The cefepime may be combined with the carrier to formulate emulsions, lotions, creams, ointments, gels, shampoos, nail lacquers, solutions, suspensions, dispersions, irrigations/baths, or pastes, for example. The cefepime may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In some examples, the cefepime comprises a commercially available cefepime, such as Cefepime Hydrochloride Injection, Powder, for Solution, supplied in 500 mg, 1 g, and 2 g vials; Cefepime Hydrochloride Injection Solution; or bulk powder. Other strengths may be used. The content of one or more vials may be added to a volume of the carrier sufficient to formulate an administration dosage of the topical composition having the desired strength of the cefepime. In some embodiments, the amount of cefepime in the administration dosage corresponds to the amount of cefepime present in a vial, or a multiple thereof.

In various embodiments, the active agent includes cefepime and an administration dosage of the topical composition comprises cefepime in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, about 4.5 g or greater, about 5 g or greater, about 5.5 g or greater, about 6 g or greater, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefepime formats such as capsules, vials for injection or solution, bulk power, solutions, topical ointments, creams, lotions, and/or gels, for example. The topical composition may be administered to the skin, vagina, nasal passage, or anal cavity. The topical composition comprising cefepime may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefepime may be applied to skin in powder or solution followed by application of a carrier or second portion of the carrier comprising gel, lotion, ointment, or cream, which may include a second portion of cefepime and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cephalexin and the carrier. The cephalenxin may be combined with the carrier to formulate emulsions, lotions, creams, ointments, gels, shampoos, nail lacquers, solutions, suspensions, dispersions, irrigations/baths, or pastes, for example. The cephalexin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the cephalexin comprises a commercially available cephalexin such as one or more cephalexin capsules comprising cephalexin monohydrate equivalent to about 250 mg, about 333 mg, about 500 mg, or about 750 mg of cephalexin and one or more of magnesium stearate, sodium starch glycolate, lactose monohydrate and/or anhydrous lactose, talc, colloidal silicone dioxide, microcrystalline cellulose, croscarmellose sodium, titanium dioxide, and/or sodium lauryl sulfate. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the cephalexin. In some embodiments, the amount of cephalexin in the administration dosage corresponds to the amount of cephalexin present in one capsule, or a multiple thereof.

In various embodiments, the active agent includes cephalexin and an administration dosage of the topical composition comprises cephalexin in an amount about 250 mg or less, about 250 mg or greater, about 330 mg or greater, about 500 mg or greater, about 750 mg or greater, about 1 g or greater, about 1.2 g or greater, about 1.5 g or greater, about 1.6 g or greater, about 1.8 g or greater, about 2 g or greater, about 2.5 g or greater, about 3 g or greater, between about 400 mg and about 700 mg, between about 700 mg and about 1 g, between about 1 g and about 1.3 g, between about 1.3 g and about 1.6 g, between about 1.6 g and about 2 g, or between about 2 g and about 3 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cephalexin formats such as vials for injection or solution, bulk power, solutions, topical ointments, creams, lotions, and/or gels, for example. The topical composition may be administered to the skin, vagina, nasal passage, or anal cavity. The topical composition comprising cephalexin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cephalexin may be applied to skin in powder or solution followed by application of a carrier or second portion of the carrier comprising gel, lotion, ointment, or cream, which may include a second portion of cephalexin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cefixime and the carrier. The cefixime may be combined with the carrier to formulate emulsions, lotions, creams, ointments, gels, shampoos, nail lacquers, solutions, suspensions, dispersions, irrigations/baths, or pastes, for example. The cefixime may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the cefixime comprises one or more commercially available cefixime oral tablets including about 400 mg cefixime and one or more of dibasic calcium phosphate, hypromellose, lactose monohydrate, magnesium stearate, microcrystalline cellulose, polyethylene glycol, pregelatinized starch, titanium dioxide, and/or triacetin. Other strengths may be used. The one or more tablets may be crushed or otherwise ground to a powder, typically prior to combining with the carrier for ease of mixing. In an above or another example, the cefixime comprises one or more commercially available cefixime oral capsules comprising about 400 mg cefixime and one or more of colloidal silicon dioxide, crospovidone, low substituted hydroxy propyl cellulose, magnesium stearate, and/or mannitol. Other strengths may be used. The one or more capsules may be preferably opened to release the contents for combining the contents with the carrier. In an above or another example, the cefixime comprises one or more commercially available chewable cefixime tablets comprising about 100 mg, about 150 mg, or about 200 mg cefixime and one or more of aspartame, colloidal silicon dioxide, crospovidone, coloring, low substituted hydroxypropyl cellulose, magnesium stearate, flavors such as fantasy flavor permaseal and/or tutti frutti flavor, and/or mannitol. Other strengths may be used. The one or more tablets may be crushed or otherwise ground to a powder, typically prior to combining with the carrier for ease of mixing. In an above or another example, the cefixime comprises one or more commercially available vials of cefixime powder for oral suspension USP comprising one or more of colloidal silicon dioxide, sodium benzoate, strawberry flavor, sucralose, sucrose, and/or xanthan gum. The one or more vials, tablets, and/or capsules, which may be the contents thereof, may be added to a volume of the carrier sufficient to formulate an administration dosage of the topical composition having the desired strength of the cefixime. In some embodiments, the amount of cefixime in the administration dosage corresponds to the amount of cefixime present in one tablet or tablet, or a multiple thereof.

In various embodiments, the active agent includes cefixime and an administration dosage of the topical composition comprises cefixime in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefixime formats such as capsules, vials for injection or solution, bulk power, solutions, topical ointments, creams, lotions, and/or gels, for example. The topical composition may be administered to the skin, vagina, nasal passage, or anal cavity. The topical composition comprising cefixime may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefixime may be applied to skin in powder or solution followed by application of a carrier or second portion of the carrier comprising gel, lotion, ointment, or cream, which may include a second portion of cefixime and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including cefixime may be administered to a body surface to treat or prevent an opportunistic infection comprising Escherichia coli, Haemophilus influenzae, Klebsiella oxytoca, Klebsiella pneumoniae, Moraxella catarrhalis, Neisseria gonorrhoeae, Proteus mirabilis, Proteus vulgaris, Providencia sp., Salmonella typhi, Serratia marcescens, Shigella sp., Peptostreptococcus, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising cefpodoxime and the carrier. The cefpodoxime may be combined with the carrier to formulate emulsions, lotions, creams, ointments, gels, shampoos, nail lacquers, solutions, suspensions, dispersions, irrigations/baths, or pastes, for example. The cefpodoxime may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the cefpodoxime comprises a commercially available cefpodoxime oral tablet comprising about cefpodoxime proxetil equivalent to 100 mg or 200 mg of cefpodoxime activity and one or more of carboxy methyl cellulose calcium, lactose monohydrate, hydroxy propyl cellulose, sodium lauryl sulfate, crospovidone, corn starch, magnesium stearate, hypromellose, titanium dioxide, coloring, and/or propylene glycol. The tablets may include film coatings that may be ground with the remainder of the tablets and combined with the carrier or removed, e.g., via brief contact with solvent and/or sifting. Other strengths may also be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the cefpodoxime. In some embodiments, the amount of cefpodoxime in the administration dosage corresponds to the amount of cefpodoxime present in one tablet, or a multiple thereof.

In various embodiments, an administration dosage of the topical composition comprises cefpodoxime in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefpodoxime formats such as capsules, vials for injection or solution, bulk power, solutions, topical ointments, creams, lotions, and/or gels, for example. The topical composition may be administered to the skin, vagina, nasal passage, or anal cavity. The topical composition comprising fluconazole may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefpodoxime may be applied to skin in powder or solution followed by application of a carrier or second portion of the carrier comprising gel, lotion, ointment, or cream, which may include a second portion of cefpodoxime and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cefotetan and the carrier. The cefotetan may be combined with the carrier to formulate emulsions, lotions, creams, ointments, gels, shampoos, nail lacquers, solutions, suspensions, dispersions, irrigations/baths, or pastes, for example. The cefotetan may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In some examples, the cefepime comprises a commercially available cefotetan, such as one or more Cefotetan for Injection vials supplied in 1 g and 2 g vials. Other strengths may be used. The contents of the one or more vials may be added to a volume of the carrier sufficient to formulate an administration dosage of the topical composition having the desired strength of the cefotetan. In some embodiments, the amount of cefotetan in the administration dosage corresponds to the amount of cefotetan present in a vial, or a multiple thereof.

In various embodiments, the active agent includes cefotetan and an administration dosage of the topical composition comprises cefotetan in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, about 4.5 g or greater, about 5 g or greater, about 5.5 g or greater, about 6 g or greater, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefotetan formats such as capsules, vials for injection or solution, bulk power, solutions, topical ointments, creams, lotions, and/or gels, for example. The topical composition may be administered to the skin, vagina, nasal passage, or anal cavity. The topical composition comprising cefotetan may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefotetan may be applied to skin in powder or solution followed by application of a carrier or second portion of the carrier comprising gel, lotion, ointment, or cream, which may include a second portion of cefotetan and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising cefprozil and the carrier. The cefprozil may be combined with the carrier to formulate emulsions, lotions, creams, ointments, gels, shampoos, nail lacquers, solutions, suspensions, dispersions, irrigations/baths, or pastes, for example. The cefprozil may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the cefprozil comprises one or more commercially available cefprozil oral tablets comprising about 250 mg or 500 mg of anhydrous cefprozil and one or more of magnesium stearate, methylcellulose, microcrystalline cellulose, sodium starch glycolate, hypromellose, polyethylene glycol 400, polysorbate 80, titanium dioxide, and/or coloring. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the cefprozil. In some embodiments, the amount of cefprozil in the administration dosage corresponds to the amount of cefprozil present in one tablet, or a multiple thereof.

In various embodiments, the active agent includes cefprozil and an administration dosage of the topical composition comprises cefprozil in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefprozil formats such as capsules, vials for injection or solution, bulk power, solutions, topical ointments, creams, lotions, and/or gels, for example. The topical composition may be administered to the skin, vagina, nasal passage, or anal cavity. The topical composition comprising cefprozil may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefprozil may be applied to skin in powder or solution followed by application of a carrier or second portion of the carrier comprising gel, lotion, ointment, or cream, which may include a second portion of cefprozil and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising ceftriaxone and the carrier. The ceftriaxone may be combined with the carrier to formulate emulsions, lotions, creams, ointments, gels, shampoos, nail lacquers, solutions, suspensions, dispersions, irrigations/baths, or pastes, for example. The ceftriaxone may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the ceftriaxone comprises one of more vials of Ceftriaxone for Injection, powder, for solution comprising ceftriaxone sodium equivalent to about 10 mg, about 500 mg, about 1 g, or about 2 g ceftriaxone. Other strengths may be used. The contents of the one or more vials may be added to a volume of the carrier sufficient to formulate an administration dosage of the topical composition having the desired strength of the ceftriaxone. In some embodiments, the amount of ceftriaxone in the administration dosage corresponds to the amount of ceftriaxone present in a vial, or a multiple thereof.

In various embodiments, the active agent includes ceftriaxone and an administration dosage of the topical composition comprises ceftriaxone in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, about 4.5 g or greater, about 5 g or greater, about 5.5 g or greater, about 6 g or greater, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available ceftriaxone formats such as capsules, vials for injection or solution, bulk power, solutions, topical ointments, creams, lotions, and/or gels, for example. The topical composition may be administered to the skin, vagina, nasal passage, or anal cavity. The topical composition comprising ceftriaxone may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including ceftriaxone may be applied to skin in powder or solution followed by application of a carrier or second portion of the carrier comprising gel, lotion, ointment, or cream, which may include a second portion of ceftriaxone and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including ceftriaxone may be administered to a body surface to treat or prevent an opportunistic infection comprising Acinetobacter baumannii, Klebsiella pneumonia, Enterococcus faecalis, Enterococcus faecium, Staphylococcus aureus, Acinetobacter baumannii, Clostridium perfringens, Enterobacter aerogenes, Enterobacter cloacae, Enterobacter sakazakii, Escherichia coli, Haemophilus ducreyi, Haemophilus influenzae, Klebsiella (Calymmatobacterium) granulomatis, Klebsiella oxytoca, Klebsiella pneumoniae, Moraxella catarrhalis, Morganella morganii, Neisseria gonorrhoeae, Neisseria meningitidis, Proteus mirabilis, Proteus vulgaris, Pseudomonas aeruginosa, Salmonella typhi, Serratia marcescens, Shigella sp., Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising cefuroxime and the carrier. The cefuroxime may be combined with the carrier to formulate emulsions, lotions, creams, ointments, gels, shampoos, nail lacquers, solutions, suspensions, dispersions, irrigations/baths, or pastes, for example. The cefuroxime may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the cefuroxime comprises one of more cefuroxime oral tablets containing equivalent of about 125 mg, about 250 mg or about 500 mg of cefuroxime as cefuroxime axetil USP and one or more of colloidal silicon dioxide, croscarmellose sodium, hydrogenated vegetable oil, hypromellose, microcrystalline cellulose, polyethylene glycol, sodium lauryl sulfate, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the cefuroxime. In some embodiments, the amount of cefuroxime in the administration dosage corresponds to the amount of cefuroxime present in one tablet, or a multiple thereof. Tablets may be film coated. The film may be ground with the remainder of the tablet or may be removed.

In various embodiments, the active agent includes cefuroxime and an administration dosage of the topical composition comprises cefuroxime in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available cefuroxime formats such as capsules, vials for injection or solution, bulk power, solutions, topical ointments, creams, lotions, and/or gels, for example. The topical composition may be administered to the skin, vagina, nasal passage, or anal cavity. The topical composition comprising cefuroxime may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including cefuroxime may be applied to skin in powder or solution followed by application of a carrier or second portion of the carrier comprising gel, lotion, ointment, or cream, which may include a second portion of cefuroxime and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including cefuroxime may be administered to a body surface to treat or prevent an opportunistic infection comprising Clostridium perfringens, Enterobacter aerogenes, Enterobacter cloacae, Enterobacter sakazakii, Escherichia coli, Haemophilus ducreyi, Haemophilus influenzae, Klebsiella (Calymmatobacterium) granulomatis, Klebsiella oxytoca, Klebsiella pneumoniae, Moraxella catarrhalis, Morganella morganii, Neisseria gonorrhoeae, Neisseria meningitidis, Proteus mirabilis, Proteus vulgaris, Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising ceftazidime and the carrier. The ceftazidime may be combined with the carrier to formulate emulsions, lotions, creams, ointments, gels, shampoos, nail lacquers, solutions, suspensions, dispersions, irrigations/baths, or pastes, for example. The ceftazidime may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the ceftazidime comprises one of more vials of Ceftazidime for Injection, powder, for solution comprising ceftazidime pentahydrate equivalent to about 1 g or 2 g ceftazidime and sodium carbonate. Other strengths may be used. The contents of the one or more vials may be added to a volume of the carrier sufficient to formulate an administration dosage of the topical composition having the desired strength of the ceftazidime. In some embodiments, the amount of ceftazidime in the administration dosage corresponds to the amount of ceftazidime present in a vial, or a multiple thereof.

In various embodiments, the active agent includes ceftazidime and an administration dosage of the topical composition comprises ceftazidime in an amount about 400 mg or less, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, about 4.5 g or greater, about 5 g or greater, about 5.5 g or greater, about 6 g or greater, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available ceftazidime formats such as capsules, tablets, bulk power, solutions, topical ointments, creams, lotions, and/or gels, for example. The topical composition may be administered to the skin, vagina, nasal passage, or anal cavity. The topical composition comprising ceftazidime may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including ceftazidime may be applied to skin in powder or solution followed by application of a carrier or second portion of the carrier comprising gel, lotion, ointment, or cream, which may include a second portion of ceftazidime and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including ceftazidime may be administered to a body surface to treat or prevent an opportunistic infection comprising Clostridium perfringens, Acinetobacter calcoaceticus, Acinetobacter lwoffii, Enterobacter aerogenes, Enterobacter cloacae, Enterobacter sakazakii, Escherichia coli, Haemophilus ducreyi, Haemophilus influenzae, Klebsiella (Calymmatobacterium) granulomatis, Klebsiella oxytoca, Klebsiella pneumoniae, Moraxella catarrhalis, Morganella morganii, Neisseria gonorrhoeae, Neisseria meningitidis, Proteus mirabilis, Proteus vulgaris, Pseudomonas aeruginosa, Salmonella typhi, Serratia marcescens, Shigella sp., Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising ciprofloxacin and the carrier. The ciprofloxacin may be combined with the carrier to formulate emulsions, lotions, creams, ointments, gels, shampoos, nail lacquers, solutions, suspensions, dispersions, irrigations/baths, or pastes, for example. The ciprofloxacin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the antibacterial agent or a carrier comprising at least a portion of the antibacterial agent may comprise a commercially available ciprofloxacin, such Ciprofloxacin Hydrochloride Solution/Drops; Ciprofloxacin Hydrochloride Tablets; Ciprofloxacin Tablets, e.g., 500 mg or 100 mg; Ciprofloxacin Hydrochloride Suspension; Ciprofloxacin Injection, USP, e.g., Ciprofloxacin Injection, USP, 20 mL, 200 mg, 1% and 40 mL or 400 mg, 1%, for intravenous injection and infusion, Premix 100 mL in 5% Dextrose, 200 mg, 0.2% and 200 mL in 5% Dextrose or 400 mg, 0.2%, for intravenous infusion; or bulk powder. The contents of the one or more vials, tablets, or injection solutions may be added to a volume of the carrier sufficient to formulate an administration dosage of the topical composition having the desired strength of the ciprofloxacin. In some embodiments, the amount of ciprofloxacin in the administration dosage corresponds to the amount of ciprofloxacin present in a vial, tablet, or solution, or a multiple thereof.

In one example, the active agent comprises an antibacterial agent comprising ciprofloxacin and a method of formulating the topical composition comprises addition of a crushed ciprofloxacin tablet to a carrier. Less than all the powder of a crushed tablet may be used when the tablet contains more ciprofloxacin than required. More than one crushed tablet may be used when the method includes formulating a topical composition comprising more ciprofloxacin than is in the tablet. The levofloxacin tablets may comprise commercially available ciprofloxacin hydrochloride 250 mg, 500 mg, or 750 mg oral tablets, for example. Other strengths may be used. In some embodiments, other strength tablets may be used. In addition to ciprofloxacin the powder may include the powder may include a glucose polymer and one or more additional components such as magnesium stearate, povidone, lactose, glycol, oxide, talc, triacetin, and/or an alcohol. In one example, the powder includes a starch such as cornstarch, sodium starch glycolate. The powder may also include magnesium stearate and/or lactose. In one embodiment, the powder includes a cellulose such as croscarmellose sodium and/or microcrystalline cellulose. The powder may also include magnesium stearate, povidone, and/or and oxide such as silicone dioxide. In an embodiment, the powder includes a cellulose such as hypromellose and/or microcrystalline cellulose. The powder may also include a starch such as cornstarch and/or sodium starch glycolate. In a further example, the powder also includes magnesium stearate. In still a further example, the powder includes polydextrose, silicon dioxide, titanium dioxide, talc, and/or triacetin. The powder may also include polyethylene glycol. In one embodiment, the powder includes a cellulose such as microcrystalline cellulose. The powder may also include a starch such as sodium starch glycolate. In a further example, the powder also includes magnesium stearate. In still a further example, the powder includes povidone, silicon dioxide, titanium dioxide, and/or polyethylene glycol. In an embodiment, the powder includes a cellulose such as croscarmellose sodium, hypromellose, and/or microcrystalline cellulose. The powder may also include a starch such as cornstarch. In a further example, the powder also includes magnesium stearate. In still a further example, the powder includes povidone, silicon dioxide, titanium dioxide, talc, and/or carnauba wax. In yet a further example, the powder includes stearic acid, succinic acid, sodium lauryl sulfate, and/or polyvinyl alcohol. The powder may also include polyethylene glycol. In one embodiment, the powder includes a cellulose such as ethylcellulose and/or hypromellose. In a further example, the powder also includes magnesium stearate. In still a further example, the powder includes povidone such as crospovidone, titanium dioxide. In yet a further example, the powder includes succinic acid. The powder may also include polyethylene glycol.

The one or more crushed ciprofloxacin tablets may be combined with the carrier. The ciprofloxacin tablets may comprise ciprofloxacin 250 mg, 500 mg, 750 mg oral tablets. The oral tablets and may be added to formulate the topical composition to include ciprofloxacin in an amount between about 0.01% and about 20% by weight, such as about 1%, about 2%, about 3%, about 4%, about 5%, less than about 5%, between about 2% and about 7%, or greater than about 10%. To formulate a topical composition comprising a desired percent by weight ciprofloxacin, the total desired weight of the topical composition is subtracted from the weight of crushed oral ciprofloxacin tablet powder needed to obtain the desired percent by weight ciprofloxacin.

In various embodiments, the active agent includes ciprofloxacin and an administration dosage of the topical composition comprises ciprofloxacin in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available ciprofloxacin formats such as capsules, vials for injection or solution, bulk power, solutions, topical ointments, creams, lotions, and/or gels, for example. The topical composition may be administered to the skin, vagina, nasal passage, or anal cavity. Additional actives, such as any active described herein, may also be added in a combined amount between 0.01% and 50% by weight, such as between about 0.01% and about 20%. The topical composition comprising ciprofloxacin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including ciprofloxacin may be applied to skin in powder or solution followed by application of a carrier or second portion of the carrier comprising gel, lotion, ointment, or cream, which may include a second portion of ciprofloxacin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including ciprofloxacin may be administered to a body surface to treat or prevent an opportunistic infection comprising Chlamydia psittaci, Bacteroides fragilis, Chlamydia pneumoniae, Chlamydia trachomatis, Mycoplasma pneumoniae, Acinetobacter lwoffii, Brucella species, Campylobacter jejuni, Enterobacter aerogenes, Enterobacter cloacae, Enterobacter sakazakii, Escherichia coli, Francisella tularensis, Haemophilus ducreyi, Haemophilus influenzae, Klebsiella (Calymmatobacterium) granulomatis, Klebsiella oxytoca, Klebsiella pneumoniae, Legionella pneumophila, Moraxella catarrhalis, Morganella morganii, Neisseria gonorrhoeae, Neisseria meningitidis, Proteus mirabilis, Proteus vulgaris, Providencia sp., Pseudomonas aeruginosa, Rickettsiae, Salmonella typhi, Serratia marcescens, Shigella sp., Vibrio cholerae, Yersinia pestis, Enterococcus faecalis, Peptostreptococcus, Staphylococcus aureus (MSSA), Staphylococcus epidermidis, Streptococcus agalactiae(B), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising clarithromycin and the carrier. The clarithromycin may be combined with the carrier to formulate emulsions, lotions, creams, ointments, gels, shampoos, nail lacquers, solutions, suspensions, dispersions, irrigations/baths, or pastes, for example. The clarithromycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the clarithromycin comprises one or more clarithromycin oral tablets containing about 250 mg or about 500 mg of clarithromycin and one or more of croscarmellose sodium, hypromellose, magnesium stearate, microcrystalline cellulose, polyethylene glycol, polysorbate 80, povidone, talc, low-substituted hydroxypropyl cellulose, colloidal silicon dioxide, titanium dioxide, hydroxypropyl cellulose, and/or coloring. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the clarithromycin. In some embodiments, the amount of clarithromycin in the administration dosage corresponds to the amount of clarithromycin present in one tablet, or a multiple thereof.

In various embodiments, the active agent includes clarithromycin and an administration dosage of the topical composition comprises clarithromycin in an amount about 100 mg or less, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available clarithromycin formats such as capsules, vials for injection or solution, bulk power, solutions, topical ointments, creams, lotions, and/or gels, for example. The topical composition may be administered to the skin, vagina, nasal passage, or anal cavity. The topical composition comprising clarithromycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including clarithromycin may be applied to skin in powder or solution followed by application of a carrier or second portion of the carrier comprising gel, lotion, ointment, or cream, which may include a second portion of clarithromycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising clindamycin and the carrier. The clindamycin may be combined with the carrier to formulate emulsions, lotions, creams, ointments, gels, shampoos, nail lacquers, solutions, suspensions, dispersions, irrigations/baths, or pastes, for example. The clindamycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the clindamycin comprises a commercially available clindamycin, such as Clindamycin Phosphate Cream; Clindamycin Phosphate Gel; Clindamycin Phosphate Suspension; Clindamycin Phosphate Injection Solution; Clindamycin Phosphate for Injection; or bulk powder. In one example, the clindamycin comprises one or more clindamycin oral capsules and/or the contents thereof containing clindamycin hydrochloride equivalent to about 75 mg, about 150 mg, or about 300 mg of clindamycin and one or more of colloidal silicon dioxide, lactose monohydrate, magnesium stearate, talc, corn starch, and/or titanium dioxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the clindamycin. In some embodiments, the amount of clindamycin in the administration dosage corresponds to the amount of clindamycin present in one capsule, or a multiple thereof.

In various embodiments, the active agent includes clindamycin and an administration dosage of the topical composition comprises clindamycin in an amount about 75 mg or less, about 75 or greater, about 100 mg or greater, about 150 mg or greater, about 200 mg or greater, about 250 mg or greater, about 350 mg or greater, about 450 mg or greater, about 550 mg or greater, about 650 mg or greater, about 850 or greater, about 1 g or greater, between about 100 mg and about 200 mg, between about 200 mg and about 300 mg, between about 300 mg and about 500 mg, or between about 500 mg and about 1 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available clindamycin formats such as capsules, vials for injection or solution, bulk power, solutions, topical ointments, creams, lotions, and/or gels, for example. The topical composition may be administered to the skin, vagina, nasal passage, or anal cavity. The topical composition comprising clindamycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including clindamycin may be applied to skin in powder or solution followed by application of a carrier or second portion of the carrier comprising gel, lotion, ointment, or cream, which may include a second portion of clindamycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including clindamycin may be administered to a body surface to treat or prevent an opportunistic infection comprising Staphylococcus aureus (MSSA), Staphylococcus aureus (MSSA, mecA positive), Staphylococcus aureus (MRSA), Staphylococcus aureus (MRSA, clindamycin-resistant), Staphylococcus epidermidis, Corynebacterium striatrum, Streptococcus pyogenes, Streptococcus sanguinis, Enterococcus faecalis, Enterococcus faecium, Escherichia coli, Pseudomonas aeruginosa, Clostridium perfringens, Bacteroides fragilis, Neisseria gonorrhoeae, Methicillin Resistant Staph aureus (MRSA), Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising colistimethate and the carrier. The colistimethate may be combined with the carrier to formulate emulsions, lotions, creams, ointments, gels, shampoos, nail lacquers, solutions, suspensions, dispersions, irrigations/baths, or pastes, for example. The colistimethate may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the colistimethate comprises one of more vials of Colistimethate for Injection, powder, for solution comprising colistimethate sodium or pentasodium colistinmethanesulfonate have about 150 mg of colistin activity. Other strengths may be used. The contents of the one or more vials may be added to a volume of the carrier sufficient to formulate an administration dosage of the topical composition having the desired strength of the colistimethate. In some embodiments, the amount of colistimethate in the administration dosage corresponds to the amount of colistimethate present in a vial, or a multiple thereof.

In various embodiments, the active agent includes colistimethate and an administration dosage of the topical composition comprises colistimethate in an amount about 50 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available colistimethate formats such as capsules, tablets, vials for injection or solution, bulk power, solutions, topical ointments, creams, lotions, and/or gels, for example. The topical composition may be administered to the skin, vagina, nasal passage, or anal cavity. The topical composition comprising colistimethate may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including colistimethate may be applied to skin in powder or solution followed by application of a carrier or second portion of the carrier comprising gel, lotion, ointment, or cream, which may include a second portion of colistimethate and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including colistimethate may be administered to a body surface to treat or prevent an opportunistic infection comprising Citrobacter freundii, Corynebacterium striatrum, Enterococcus faecalis, Escherichia coli, Streptococcus pyogenes, Streptococcus sanguinis (Viridans Group), Klebsiella pneumoniae, Morganella morganii, Proteus mirabilis, Pseudomonas aeruginosa, Staphylococcus aureus (MSSA), Staphylococcus aureus (MRSA), Staphylococcus epidermidis, Acinetobacter baumannii, Acinetobacter calcoaceticus, Acinetobacter lwoffii, Enterobacter aerogenes, Enterobacter cloacae, Enterobacter sakazakii, Escherichia coli, Klebsiella (Calymmatobacterium) granulomatis, Klebsiella oxytoca, Klebsiella pneumoniae, Pseudomonas aeruginosa, and/or Staphylococcus epidermidis. The treatment with the topical composition may also prevent or reduce an ability of such bacteria from proliferating to significantly infect the body surface or adjacent tissue.

In one embodiment, the method includes combining an active agent comprising one or more antibacterials comprising doxycycline and the carrier. The doxycycline may be combined with the carrier to formulate emulsions, lotions, creams, ointments, gels, shampoos, nail lacquers, solutions, suspensions, dispersions, irrigations/baths, or pastes, for example. The doxycycline may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the antibacterial agent or a carrier comprising at least a portion of the antibacterial agent may comprise a commercially available doxycycline, such as Doxycycline Hyclate tablets; Doxycycline Hyclate Tablets; Doxycycline Hyclate Pellets; Doxycycline for Suspension; Doxycycline Hyclate Powder for Suspension; or bulk powder. In one example, the doxycycline comprises one or more doxycycline hyclate oral tablets containing doxycycline hyclate equivalent to 50 mg or 100 mg of doxycycline and one or more of magnesium stearate, powdered/microcrystalline cellulose, sodium lauryl sulfate, titanium dioxide, anhydrous lactose, colloidal silicon dioxide, methylcellulose, polyethylene glycol, sodium starch glycolate, and/or stearic acid. Other strengths may be used. The one or more capsules may be preferably opened to release the contents for combining the contents with the carrier. The one or more tablets may be added to a volume of the carrier sufficient to formulate an administration dosage of the topical composition having the desired strength of the doxycycline. In some embodiments, the amount of doxycycline in the administration dosage corresponds to the amount of doxycycline present in one tablet, or a multiple thereof.

In various embodiments, the active agent includes doxycycline and an administration dosage of the topical composition comprises doxycycline in an about 100 mg or less, about 50 mg or greater, about 100 mg or greater, about 150 mg or greater, about 200 mg or greater, about 250 mg or greater, about 300 mg or greater, between about 50 mg and about 100 mg, between about 70 mg and about 150 mg, between about 100 mg and about 200 mg, between about 150 mg and about 250 mg, or between about 200 mg and about 300 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available doxycycline formats such as capsules, vials for injection or solution, bulk power, solutions, topical ointments, creams, lotions, and/or gels, for example. The topical composition may be administered to the skin, vagina, nasal passage, or anal cavity. The topical composition comprising doxycycline may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including doxycycline may be applied to skin in powder or solution followed by application of a carrier or second portion of the carrier comprising gel, lotion, ointment, or cream, which may include a second portion of doxycycline and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including doxycycline may be administered to a body surface to treat or prevent an opportunistic infection comprising Clostridium perfringens, Bacteroides fragilis, Chlamydia pneumoniae, Chlamydia psittaci, Chlamydia trachomatis, Mycoplasma pneumoniae, Brucella species, Francisella tularensis, Haemophilus influenzae, Klebsiella (Calymmatobacterium) granulomatis, Legionella pneumophila, Moraxella catarrhalis, Neisseria gonorrhoeae, Neisseria meningitidis, Rickettsiae, Salmonella typhi, Shigella sp., Vibrio cholerae, Yersinia pestis, Enterococcus faecalis, Methicillin Resistant Staph aureus (MRSA), Peptostreptococcus, Staphylococcus aureus (MSSA), Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising erythromycin and the carrier. The erythromycin may be combined with the carrier to formulate emulsions, lotions, creams, ointments, gels, shampoos, nail lacquers, solutions, suspensions, dispersions, irrigations/baths, or pastes, for example. The erythromycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the erythromycin comprises a commercially available erythromycin comprising one or more erythromycin tablets containing either 250 mg or 500 mg of erythromycin base and one or more of colloidal silicon dioxide, croscarmellose sodium, crospovidone, coloring, hydroxypropyl cellulose, hypromellose, hydroxypropyl methylcellulose phthalate, magnesium stearate, microcrystalline cellulose, povidone, polyethylene glycol, propylene glycol, sodium citrate, sodium hydroxide, sorbic acid, sorbitan monooleate, talc, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the erythromycin. In some embodiments, the amount of erythromycin in the administration dosage corresponds to the amount of erythromycin present in one tablet, or a multiple thereof.

In various embodiments, the active agent includes erythromycin and an administration dosage of the topical composition comprises erythromycin in an amount about 100 mg or less, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available erythromycin formats such as capsules, vials for injection or solution, bulk power, solutions, topical ointments, creams, lotions, and/or gels, for example. The topical composition may be administered to the skin, vagina, nasal passage, or anal cavity. The topical composition comprising erythromycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including erythromycin may be applied to skin in powder or solution followed by application of a carrier or second portion of the carrier comprising gel, lotion, ointment, or cream, which may include a second portion of erythromycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including erythromycin may be administered to a body surface to treat or prevent an opportunistic infection comprising Staphylococcus aureus (MSSA), Staphylococcus aureus (MSSA, mecA positive), Staphylococcus aureus (MRSA), Staphylococcus aureus (MRSA, clindamycin-resistant), Staphylococcus epidermidis, Corynebacterium striatrum, Streptococcus pyogenes, Streptococcus sanguinis, Enterococcus faecalis, Enterococcus faecium, Escherichia coli, Pseudomonas aeruginosa, Clostridium perfringens, Chlamydia pneumoniae, Chlamydia trachomatis, Mycoplasma pneumoniae, Bordetella pertussis, Campylobacter jejuni, Haemophilus ducreyi, Klebsiella (Calymmatobacterium) granulomatis, Legionella pneumophila, Moraxella catarrhalis, Salmonella typhi, Vibrio cholerae, Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising gentamycin and the carrier. The gentamycin may be combined with the carrier to formulate emulsions, lotions, creams, ointments, gels, shampoos, nail lacquers, solutions, suspensions, dispersions, irrigations/baths, or pastes, for example. The gentamicin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the gentamicin comprises bulk powder or a compounded capsule containing between about 50 mg and about 150 mg gentamicin, such as about 80 mg gentamycin. The method may include opening the capsule or otherwise releasing the gentamycin for combining with the carrier.

In various embodiments, the active agent includes gentamicin and an administration dosage of the topical composition comprises gentamicin in an amount about 100 mg or less, about 50 mg or greater, about 100 mg or greater, about 150 mg or greater, about 200 mg or greater, about 300 mg or greater, about 400 mg or greater, about 500 mg or greater, about 600 mg or greater, between about 50 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available gentamycin formats such as capsules, vials for injection or solution, bulk power, solutions, topical ointments, creams, lotions, and/or gels, for example. The topical composition may be administered to the skin, vagina, nasal passage, or anal cavity. The topical composition comprising gentamycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including gentamycin may be applied to skin in powder or solution followed by application of a carrier or second portion of the carrier comprising gel, lotion, ointment, or cream, which may include a second portion of gentamycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including gentamycin may be administered to a body surface to treat or prevent an opportunistic infection comprising Brucella species, Campylobacter jejuni, Enterobacter aerogenes, Enterobacter cloacae, Enterobacter sakazakii, Escherichia coli, Francisella tularensis, Klebsiella (Calymmatobacterium) granulomatis, Klebsiella oxytoca, Legionella pneumophila, Morganella morganii, Proteus mirabilis, Proteus vulgaris, Providencia sp., Pseudomonas aeruginosa, Salmonella typhi, Serratia marcescens, Shigella sp., Yersinia pestis, Enterococcus faecalis, Enterococcus faecium, Methicillin Resistant Staph aureus (MRSA), Staphylococcus aureus (MSSA), Staphylococcus epidermidis, Streptococcus agalactiae(B), Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising isoniazid and the carrier. The isoniazid may be combined with the carrier to formulate emulsions, lotions, creams, ointments, gels, shampoos, nail lacquers, solutions, suspensions, dispersions, irrigations/baths, or pastes, for example. The isoniazid may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the isoniazid comprises a commercially available isoniazid comprising one or more isoniazid tablets containing either about 100 mg or about 300 mg isoniazid and one or more of calcium sulfate, pregelatinized starch, croscarmellose sodium, povidone, calcium stearate, anhydrous lactose, calcium stearate, colloidal silicon dioxide, microcrystalline cellulose, stearic acid, crospovidone, hydrogenated vegetable oil, pregelatinized starch, and/or talc. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the isoniazid. In some embodiments, the amount of isoniazid in the administration dosage corresponds to the amount of isoniazid present in one tablet, or a multiple thereof.

In various embodiments, the active agent includes isoniazid and an administration dosage of the topical composition comprises isoniazid in an amount about 100 mg or less, about 100 mg or greater, about 150 mg or greater, about 200 mg or greater, about 250 mg or greater, about 350 mg or greater, about 450 mg or greater, about 550 mg or greater, about 650 mg or greater, about 850 or greater, about 1 g or greater, between about 100 mg and about 200 mg, between about 200 mg and about 300 mg, between about 300 mg and about 500 mg, or between about 500 mg and about 1 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available isoniazid formats such as capsules, vials for injection or solution, bulk power, solutions, topical ointments, creams, lotions, and/or gels, for example. The topical composition may be administered to the skin, vagina, nasal passage, or anal cavity. The topical composition comprising isoniazid may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including isoniazid may be applied to skin in powder or solution followed by application of a carrier or second portion of the carrier comprising gel, lotion, ointment, or cream, which may include a second portion of isoniazid and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising levofloxacin and the carrier. The levofloxacin may be combined with the carrier to formulate emulsions, lotions, creams, ointments, gels, shampoos, nail lacquers, solutions, suspensions, dispersions, irrigations/baths, or pastes, for example. The levofloxacin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the levofloxacin comprises Levofloxacin for Injection, which may be supplied in single-use vials containing a concentrated solution with the equivalent of 500 mg of levofloxacin USP in 20 mL vials and 750 mg of levofloxacin USP in 30 mL vials; Levofloxacin Solution/Drops; Levofloxacin Tablet 250 mg, 500 mg, 750 mg; or bulk powder. Other strengths may be used. In one example, the topical composition may comprise between about 0.01% to about 40% levofloxacin by weight. An administration dosage may include about 10 mg to about 900 mg levofloxacin or more. A method of formulating a topical composition comprising levofloxacin may include addition of a crushed levofloxacin tablet to a carrier. Less than all the powder of a crushed tablet may be used when the tablet contains more levofloxacin than required. More than one crushed tablet may be used when the method includes formulating a topical composition comprising more levofloxacin than is in the tablet. The levofloxacin tablets may comprise commercially available levofloxacin 250 mg, 500 mg, or 750 mg oral tablets, for example. Other strengths may also be used. In some embodiments, other strength tablets may be used. In some embodiments, the levofloxacin tablets may comprise a film coating. In addition to levofloxacin the powder may include a glucose polymer comprising a starch and/or a cellulose and one or more additional components such as magnesium stearate, povidone, lactose, glycol, oxide, talc, triacetin, an alcohol, or combination thereof. In one example, the powder includes cornstarch, croscarmellose sodium, hypromellose, microcrystalline cellulose, magnesium stearate, polyethylene glycol, povidone and titanium dioxide. In another example, the powder includes sodium starch glycolate, hypromellose, microcrystalline cellulose, magnesium stearate, polyethylene glycol, propylene glycol, povidone, polysorbate, colloidal silicon dioxide, and titanium dioxide. In still another example, the powder includes hypromellose, microcrystalline cellulose, magnesium stearate, polyethylene glycol 6000, crospovidone, talc, and titanium dioxide. In yet another example, the powder includes sodium starch glycolate, croscarmellose sodium, hydroxypropyl cellulose, hypromellose, polyethylene glycol 400, povidone K 30, glycerol behenate, lactose monohydrate, colloidal silicon dioxide, titanium dioxide, ferric oxide, and talc. The one or more tablets may be crushed or otherwise ground to a powder, typically prior to combining with the carrier for ease of mixing. In one example, the oral tablets may be crushed and combined with the carrier to formulate a topical composition comprising between 0.01% and 20% by weight, such as about 1%, about 2%, about 3%, about 4%, about 5%, less than about 5%, between about 2% and about 7%, or greater than about 10% levofloxacin by weight. In one example, the method may include combining about 125 mg levofloxacin bulk powder, which may be provided in a compounded capsule, with an amount of carrier to make an administration dosage. The contents of one or more vials, capsules, or tablets may be added to a volume of the carrier sufficient to formulate an administration dosage of the topical composition having the desired strength of the levofloxacin. In some embodiments, the amount of levofloxacin in the administration dosage corresponds to the amount of levofloxacin present in a vial, tablet, or capsule, or a multiple thereof.

In various embodiments, the active agent includes levofloxacin and an administration dosage of the topical composition comprises levofloxacin in an amount about 100 mg or less, about 100 mg or greater, about 150 mg or greater, about 250 mg or greater, about 300 mg or greater, about 400 mg or greater, about 500 mg or greater, about 600 mg or greater, about 750 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available levofloxacin formats such as capsules, vials for injection or solution, bulk power, solutions, topical ointments, creams, lotions, and/or gels, for example. The topical composition may be administered to the skin, vagina, nasal passage, or anal cavity. The topical composition comprising levofloxacin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including levofloxacin may be applied to skin in powder or solution followed by application of a carrier or second portion of the carrier comprising gel, lotion, ointment, or cream, which may include a second portion of levofloxacin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including levofloxacin may be administered to a body surface to treat or prevent an opportunistic infection comprising Escherichia coli, Staphylococcus aureus, Klebsiella pneumoniae, Clostridium perfringens, Chlamydia pneumoniae, Chlamydia psittaci, Chlamydia trachomatis, Mycoplasma pneumoniae, Acinetobacter lwoffii, Brucella species, Enterobacter aerogenes, Enterobacter cloacae, Enterobacter sakazakii, Escherichia coli, Haemophilus ducreyi, Haemophilus influenzae, Klebsiella oxytoca, Klebsiella pneumoniae, Legionella pneumophila, Moraxella catarrhalis, Morganella morganii, Neisseria gonorrhoeae, Neisseria meningitidis, Proteus mirabilis, Proteus vulgaris, Providencia sp., Pseudomonas aeruginosa, Salmonella typhi, Serratia marcescens, Shigella sp., Vibrio cholerae, Corynebacterium jeikeium, Enterococcus faecalis, Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one example, the active agent comprises an antibacterial agent comprising levofloxacin and the method of formulating the topical composition comprises addition of a crushed levofloxacin tablet to a carrier. The levofloxacin tablets may comprise commercially available levofloxacin 250 mg, 500 mg, 750 mg oral tablets. To formulate a topical composition comprising a desired percent by weight levofloxacin, the total desired weight of the topical composition is subtracted from the weight of crushed oral levofloxacin tablet powder needed to obtain the desired percent by weight levofloxacin in a manner similar to that described above with respect to voriconazole. The carrier may comprise a suitable carrier or carrier components thereof selected to formulate a topical composition comprising a format selected from a cream, gel, lotion, ointment, emulsion (oil-in-water or water-in-oil), foam, solution, dispersion, or powder, for example, suitable for topical application. In one example, the topical composition may be formulated for administration in a vaginal or anal orifice. In one example, the topical composition comprises a solution or suspension for administration in a hand or footbath or by irrigation. In another example, the topical composition comprises a nail lacquer for administration to nails. Further to the above, the carrier may comprise components described herein for formulating the formats above or elsewhere herein. In an above or another example, the carrier comprises a commercially available composition comprising abase, such as those described herein. In an above or another example, the carrier may comprise a commercially available medicated composition, such as those described herein. Additional active agents may include one or more antifungal actives, antibacterial actives, or both. Such additional active agent may be present in a combined amount between 0.01% and 20% by weight, such as between about 0.01% and about 5%. Additionally or alternatively, additional actives may include other active agents such as one or more active agents selected from an antiviral agent, an anti-inflammatory agent, a steroid, an anti-allergy agent, an antidepressant agent, a stimulant agent, a disinfectant agent, an anticonvulsant agent, a local anesthetic agent, an anticonvulsant agent, a nerve depressant agent, a muscle relaxant agent, a NMDA (N-Methyl-D-aspartate) receptor antagonist agent, an opiate or opioid agonist agent, an NSAID agent, an analgesic agent, a keratolytic agent, or combination thereof. Such additional active agents may be present in a combined amount between 0.01% and 25% by weight, such as between about 1% and about 10%.

In one embodiment, the method includes combining an active agent comprising one or more antibacterials comprising linezolid and the carrier. The linezolid may be combined with the carrier to formulate emulsions, lotions, creams, ointments, gels, shampoos, nail lacquers, solutions, suspensions, dispersions, irrigations/baths, or pastes, for example. The linezolid may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the active agent comprises an antibacterial agent comprising linezolid and a method of formulating the topical composition comprises addition of a crushed linezolid tablet to a carrier. Less than all the powder of a crushed tablet may be used when the tablet contains more linezolid than required. More than one crushed tablet may be used when the method includes formulating a topical composition comprising more linezolid than is in the tablet. The linezolid tablets may comprise commercially available linezolid 600 mg oral tablets, for example. In some embodiments, other strength tablets may be used. In addition to linezolid the powder may include a glucose polymer comprising a starch and/or a cellulose and one or more additional components such as magnesium stearate, povidone, lactose, glycol, oxide, talc, triacetin, an alcohol, or combination thereof. In various examples, the powder includes a starch and a cellulose. In other embodiments, the powder does not include a starch. In one example, the powder includes croscarmellose sodium, diethyl phthalate, ethyl cellulose, pregelatinized starch, sodium starch glycolate, mannitol, colloidal silicon dioxide, povidone, copovidone, cospovidine, sodium stearyl fumarate, hypromellose, polyethylene glycol, titanium dioxide, magnesium stearate, microcrystalline cellulose, talc, hydroxypropyl cellulose, polydextrose, triacetin, carnauba wax, lactose monohydrate, polacrilin potassium, sodium lauryl sulfate, or a combination thereof. In one example, the powder includes a starch comprising pregelatinized starch, a cellulose comprising hypromellose, a sugar alcohol comprising mannitol, a glycol comprising polyethylene glycol, an oxide comprising titanium dioxide and/or colloidal silicon dioxide, a povidone comprising copovidone, and sodium stearyl fumarate. In another example, the powder includes a cellulose comprising croscarmellose sodium, ethyl cellulose, hypromellose, and/or microcrystalline cellulose, magnesium stearate, povidone, an oxide comprising silicon dioxide and/or titanium dioxide, talc, and diethyl phthalate. In yet another example, the powder comprises a cellulose comprising microcrystalline cellulose, hydroxypropyl cellulose, and/or hypromellose, polydextrose, magnesium stearate, crospovidone, polyethylene glycol, titanium dioxide, and triacetin. In one embodiment, the powder comprises a starch comprising cornstarch and/or sodium starch glycolate, a cellulose comprising microcrystalline cellulose, hypromellose, and/or hydroxypropylcellulose, magnesium stearate, polyethylene glycol, titanium dioxide, and carnauba wax. In another example, the powder comprises hypromellose, lactose monohydrate, magnesium stearate, polyethylene glycol, colloidal silicon dioxide, titanium dioxide, polacrilin potassium, and carnauba wax. In one embodiment, the powder comprises a cellulose comprising croscarmellose sodium and/or hypromellose, lactose monohydrate, magnesium stearate, polyethylene glycol 400, povidone and titanium dioxide. In another embodiment, the powder comprises a cellulose comprising croscarmellose sodium and/or microcrystalline cellulose, polydextrose, magnesium stearate, polyethylene glycol, sodium lauryl sulfate, colloidal silicon dioxide, titanium dioxide and triacetin. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the linezolid. In some embodiments, the amount of linezolid in the administration dosage corresponds to the amount of linezolid present in one tablet, or a multiple thereof.

In various embodiments, the active agent includes linezolid and an administration dosage of the topical composition comprises linezolid in an amount about 100 mg or less, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 100 mg and about 300 mg, between about 300 mg and about 500 mg, between about 500 mg and about 800 mg, between about 800 mg and about 1.2 g, between about 1.2 g and about 1.6 g, between about 1.4 g and about 1.8 g, or between about 1.6 g and about 1.8 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available linezolid formats such as capsules, vials for injection or solution, bulk power, solutions, topical ointments, creams, lotions, and/or gels, for example. The topical composition may be administered to the skin, vagina, nasal passage, or anal cavity. The topical composition comprising linezolid may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein in a manner described above or elsewhere herein. For example, the composition including one or more actives including linezolid may be applied to skin in powder or solution followed by application of a carrier or second portion of the carrier comprising gel, lotion, ointment, or cream, which may include a second portion of linezolid and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including linezolid may be administered to a body surface to treat or prevent an opportunistic infection comprising Clostridium perfringens, Corynebacterium jeikeium, Enterococcus faecalis, Enterococcus faecium, Methicillin Resistant Staph aureus (MRSA), Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising meropenem and the carrier. The meropenem may be combined with the carrier to formulate emulsions, lotions, creams, ointments, gels, shampoos, nail lacquers, solutions, suspensions, dispersions, irrigations/baths, or pastes, for example. In various embodiments, an administration dosage of the topical composition comprises meropenem in an amount about 50 mg or less, about 50 mg, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 20 mg and about 50 mg, about 50 mg and about 100 mg, between about 100 mg and about 300 mg, between about 300 mg and about 500 mg, between about 500 mg and about 800 mg, between about 800 mg and about 1.1 g, or between about 1.1 g and about 1.5 g. The meropenem may comprise commercially available tablets, capsules, vials for injection, e.g., or solution, bulk power, solutions, topical ointments, creams, lotions, and/or gels, for example. One embodiment, the meropenem comprises Meropenem for Injection, powder, 500 mg or 1 mg vial. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available meropenem formats such as vials for injection or solution, bulk power, solutions, topical ointments, creams, lotions, and/or gels, for example. The topical composition may be administered to the skin, vagina, nasal passage, or anal cavity. The topical composition comprising meropenem may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein in a manner described above or elsewhere herein. For example, the composition including one or more actives including meropenem may be applied to skin in powder or solution followed by application of a carrier or second portion of the carrier comprising gel, lotion, ointment, or cream, which may include a second portion of meropenem and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including meropenem may be administered to a body surface to treat or prevent an opportunistic infection comprising Bacteroides fragilis, Clostridium difficile, Acinetobacter baumannii, Acinetobacter calcoaceticus, Acinetobacter lwoffii, Enterobacter aerogenes, Enterobacter cloacae, Enterobacter sakazakii, Escherichia coli, Haemophilus ducreyi, Haemophilus influenzae, Klebsiella (Calymmatobacterium) granulomatis, Klebsiella oxytoca, Klebsiella pneumoniae, Morganella morganii, Neisseria gonorrhoeae, Neisseria meningitidis, Proteus mirabilis, Proteus vulgaris, Providencia sp., Pseudomonas aeruginosa, Salmonella typhi, Serratia marcescens, Shigella sp., Enterococcus faecalis, Enterococcus faecium, Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising moxifloxacin and the carrier. The moxifloxacin may be combined with the carrier to formulate emulsions, lotions, creams, ointments, gels, shampoos, nail lacquers, solutions, suspensions, dispersions, irrigations/baths, or pastes, for example. The moxifloxacin may comprise commercially available tablets, capsules, vials for injection or solution, bulk power, solutions, topical ointments, creams, lotions, and/or gels, for example. In one formulation, the moxifloxacin comprises moxifloxacin hydrochloride tablets, such as 400 mg tablets, comprising moxifloxacin hydrochloride equivalent to 400 mg moxifloxacin and sodium croscarmellose, copovidone, microcrystalline cellulose, pregelatinized starch, talc, colloidal silicon dioxide, magnesium stearate, polyvinyl alcohol-part hydrolyzed, titanium dioxide, macrogol/peg, and/or ferric oxide red. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In various embodiments, the active agent includes moxifloxacin and an administration dosage of the topical composition comprises moxifloxacin in an amount about 100 mg or less, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available moxifloxacin formats such as vials for injection or solution, bulk power, solutions, topical ointments, creams, lotions, and/or gels, for example. The topical composition may be administered to the skin, vagina, nasal passage, or anal cavity. The topical composition comprising moxifloxacin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein in a manner described above or elsewhere herein. For example, the composition including one or more actives including moxifloxacin may be applied to skin in powder or solution followed by application of a carrier or second portion of the carrier comprising gel, lotion, ointment, or cream, which may include a second portion of moxifloxacin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including moxifloxacin may be administered to a body surface to treat or prevent an opportunistic infection comprising Bacteroides fragilis, Clostridium difficile, Chlamydia pneumoniae, Chlamydia psittaci, Chlamydia trachomatis, Mycoplasma pneumoniae, Acinetobacter lwoffii, Brucella species, Haemophilus ducreyi, Haemophilus influenzae, Klebsiella (Calymmatobacterium) granulomatis, Legionella pneumophila, Neisseria gonorrhoeae, Neisseria meningitidis, Proteus mirabilis, Proteus vulgaris, Providencia sp., Pseudomonas aeruginosa, Rickettsiae, Salmonella typhi, Serratia marcescens, Shigella sp., Vibrio cholerae, Corynebacterium jeikeium, Enterococcus faecalis, Enterococcus faecium, Methicillin Resistant Staph aureus (MRSA), Peptostreptococcus, Staphylococcus aureus (MSSA), Staphylococcus epidermidis, Streptococcus agalactiae(B), Streptococcus pneumoniae, Streptococcus pyogenes(A), and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising mupirocin and the carrier. The mupirocin may be combined with the carrier to formulate emulsions, lotions, creams, ointments, gels, shampoos, nail lacquers, solutions, suspensions, dispersions, irrigations/baths, or pastes, for example. The mupirocin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining between about 10 mg and about 300 mg mupirocin with the carrier to formulate an administration dosage. For instance, one or more compounded capsules comprising about 20 mg or about 200 mg mupirocin may be combined with the carrier. The compounded capsule may be opened or otherwise manipulated to release the contents for combining with the carrier.

In various embodiments, the active agent includes mupirocin and an administration dosage of the topical composition comprises mupirocin in an amount about 20 mg or less, about 20 mg or greater, about 40 mg or greater, about 60 mg or greater, about 75 or greater, about 100 mg or greater, about 150 mg or greater, about 200 mg or greater, about 250 mg or greater, about 350 mg or greater, about 450 mg or greater, about 550 mg or greater, about 650 mg or greater, about 850 or greater, about 1 g or greater, between about 10 mg and about 40 mg, between about 40 mg and about 100 mg, between about 100 mg and about 200 mg, between about 200 mg and about 300 mg, between about 300 mg and about 500 mg, or between about 500 mg and about 1 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available mupirocin formats such as capsules, vials for injection or solution, bulk power, solutions, topical ointments, creams, lotions, and/or gels, for example. The topical composition may be administered to the skin, vagina, nasal passage, or anal cavity. The topical composition comprising mupirocin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein in a manner described above or elsewhere herein. For example, the composition including one or more actives including mupirocin may be applied to skin in powder or solution followed by application of a carrier or second portion of the carrier comprising gel, lotion, ointment, or cream, which may include a second portion of mupirocin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including mupirocin may be administered to a body surface to treat or prevent an opportunistic infection comprising Acinetobacter baumannii, Corynebacterium striatum, Enterobacter cloacae complex, Enterococcus faecalis, Group A Streptococcus pyogenes, Group V Streptococcus sanguinis, Klebsiella pneumonia, Moraxella catarrhalis, Pseudomonas aeruginosa, Staphylococcus aureus (MSSA), Staphylococcus aureus (MRSA), Staphylococcus epidermidis, Methicillin Resistant Staph aureus (MRSA), Staphylococcus aureus (MSSA), and/or Streptococcus pyogenes(A). The treatment with the topical composition may also prevent or reduce an ability of such bacteria from proliferating to significantly infect the body surface or adjacent tissue.

In one embodiment, the method includes combining an active agent comprising one or more antibacterials comprising nafcillin and the carrier. The nafcillin may be combined with the carrier to formulate emulsions, lotions, creams, ointments, gels, shampoos, nail lacquers, solutions, suspensions, dispersions, irrigations/baths, or pastes, for example. The nafcillin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining a vial of Nafcillin for Injection containing nafcillin sodium as the monohydrate equivalent to either 1 g or 2 g of nafcillin per vial, buffered with 40 mg sodium citrate per gram. Other strengths may be used. The contents of the one or more vials may be added to a volume of the carrier sufficient to formulate an administration dosage of the topical composition having the desired strength of the nafcillin. In some embodiments, the amount of nafcillin in the administration dosage corresponds to the amount of nafcillin present in a vial, or a multiple thereof.

In various embodiments, the active agent includes nafcillin and an administration dosage of the topical composition comprises nafcillin in an amount about 200 mg or less, about 200 mg or greater, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, about 4.5 g or greater, about 5 g or greater, about 5.5 g or greater, about 6 g or greater, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available nafcillin formats such as capsules, vials for injection or solution, bulk power, solutions, topical ointments, creams, lotions, and/or gels, for example. The topical composition may be administered to the skin, vagina, nasal passage, or anal cavity. The topical composition comprising nafcillin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including nafcillin may be applied to skin in powder or solution followed by application of a carrier or second portion of the carrier comprising gel, lotion, ointment, or cream, which may include a second portion of nafcillin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising nitrofurantoin and the carrier. The nitrofurantoin may be combined with the carrier to formulate emulsions, lotions, creams, ointments, gels, shampoos, nail lacquers, solutions, suspensions, dispersions, irrigations/baths, or pastes, for example. The nitrofurantoin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining one or more nitrofurantoin capsules and/or the contents thereof containing either about 25 mg, about 50 mg, or about 100 mg nitrofurantoin monohydrate 75%/microcrystals 25% and one or more of carbomer 934P, colloidal silicon dioxide, corn starch, compressible sugar, lactose monohydrate, magnesium stearate, povidone, talc, and/or titanium dioxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the nitrofurantoin. In some embodiments, the amount of nitrofurantoin in the administration dosage corresponds to the amount of nitrofurantoin present in one capsule, or a multiple thereof.

In various embodiments, the active agent includes nitrofurantoin and an administration dosage of the topical composition comprises nitrofurantoin in an amount about 10 mg or less, about 10 mg or greater, about 25 mg or greater, about 50 mg or greater, about 60 mg or greater, about 75 or greater, about 100 mg or greater, about 150 mg or greater, about 200 mg or greater, about 300 mg or greater, about 400 mg or greater, about 500 mg or greater, about 600 mg or greater, between about 10 mg and about 25 mg, between about 25 mg and about 50 mg, between about 50 mg and about 75 mg, between about 75 mg and about 100 mg, between about 100 mg and about 150 mg, between about 150 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available nitrofurantoin formats such as capsules, vials for injection or solution, bulk power, solutions, topical ointments, creams, lotions, and/or gels, for example. The topical composition may be administered to the skin, vagina, nasal passage, or anal cavity. The topical composition comprising nitrofurantoin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including nitrofurantoin may be applied to skin in powder or solution followed by application of a carrier or second portion of the carrier comprising gel, lotion, ointment, or cream, which may include a second portion of nitrofurantoin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including nitrofurantoin may be administered to a body surface to treat or prevent an opportunistic infection comprising Citrobacter freundii, Corynebacterium striatrum, Escherichia coli, Streptococcus pyogenes, Streptococcus sanguinis (Viridans Group), Klebsiella pneumoniae, Morganella morganii, Proteus mirabilis, Pseudomonas aeruginosa, Staphylococcus aureus (MSSA), Staphylococcus aureus (MRSA), Staphylococcus epidermidis, Enterobacter aerogenes, Enterobacter cloacae, Enterobacter sakazakii, Escherichia coli, Klebsiella oxytoca, Klebsiella pneumoniae, Enterococcus faecalis, and/or Enterococcus faecium. The treatment with the topical composition may also prevent or reduce an ability of such bacteria from proliferating to significantly infect the body surface or adjacent tissue.

In one embodiment, the method includes combining an active agent comprising one or more antibacterials comprising ofloxacin and the carrier. The ofloxacin may be combined with the carrier to formulate emulsions, lotions, creams, ointments, gels, shampoos, nail lacquers, solutions, suspensions, dispersions, irrigations/baths, or pastes, for example. The ofloxacin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining one or more ofloxacin oral tablets comprising either about 200 mg or about 400 mg ofloxacin and one or more of lactose monohydrate, pregelatinized maize starch, hydroxy propyl methyl cellulose, talc, magnesium stearate, polyethylene glycol, sodium starch glycolate, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the ofloxacin. In some embodiments, the amount of ofloxacin in the administration dosage corresponds to the amount of ofloxacin present in one tablet, or a multiple thereof.

In various embodiments, the active agent includes ofloxacin and an administration dosage of the topical composition comprises ofloxacin in an amount about 100 mg or less, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available ofloxacin formats such as capsules, vials for injection or solution, bulk power, solutions, topical ointments, creams, lotions, and/or gels, for example. The topical composition may be administered to the skin, vagina, nasal passage, or anal cavity. The topical composition comprising ofloxacin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including ofloxacin may be applied to skin in powder or solution followed by application of a carrier or second portion of the carrier comprising gel, lotion, ointment, or cream, which may include a second portion of ofloxacin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising tetracycline and the carrier. The tetracycline may be combined with the carrier to formulate emulsions, lotions, creams, ointments, gels, shampoos, nail lacquers, solutions, suspensions, dispersions, irrigations/baths, or pastes, for example. The tetracycline may comprise commercially available tablets, capsules, vials for injection, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining one more tetracycline oral capsules or the contents of one or more tetracycline oral capsules with the carrier. The capsules may contain about 250 mg or about 500 mg tetracycline hydrochloride and one or more of light mineral oil, lactose monohydrate, colloidal silicon dioxide, steric acid, and/or magnesium stearate. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the tetracycline. In some embodiments, the amount of tetracycline in the administration dosage corresponds to the amount of tetracycline present in one capsule, or a multiple thereof.

In various embodiments, the active agent includes tetracycline and an administration dosage of the topical composition comprises tetracycline in an amount about 100 mg or less, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available tetracycline formats such as tablets, vials for injection, bulk power, solutions, topical ointments, creams, lotions, and/or gels, for example. The topical composition may be administered to the skin, vagina, nasal passage, or anal cavity. The topical composition comprising tetracycline may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including tetracycline may be applied to skin in powder or solution followed by application of a carrier or second portion of the carrier comprising gel, lotion, ointment, or cream, which may include a second portion of tetracycline and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including tetracycline may be administered to a body surface to treat or prevent an opportunistic infection comprising Bacteroides fragilis, Clostridium perfringens, Chlamydia pneumoniae, Chlamydia psittaci, Chlamydia trachomatis, Mycoplasma pneumoniae, Acinetobacter baumannii, Acinetobacter calcoaceticus, Acinetobacter lwoffii, Brucella species, Campylobacter jejuni, Escherichia coli, Francisella tularensis, Haemophilus influenzae, Klebsiella (Calymmatobacterium) granulomatis, Legionella pneumophila, Moraxella catarrhalis, Neisseria gonorrhoeae, Rickettsiae, Shigella sp., Vibrio cholerae, Yersinia pestis, Enterococcus faecalis, Enterococcus faecium, Methicillin Resistant Staph aureus (MRSA), Peptostreptococcus, Staphylococcus aureus (MSSA), Staphylococcus epidermidis, Streptococcus pneumoniae, and/or Viridans group streptococci. The treatment with the topical composition may also prevent or reduce an ability of such bacteria from proliferating to significantly infect the body surface or adjacent tissue.

In one embodiment, the method includes combining an active agent comprising one or more antibacterials comprising tobramycin and the carrier. The tobramycin may be combined with the carrier to formulate emulsions, lotions, creams, ointments, gels, shampoos, nail lacquers, solutions, suspensions, dispersions, irrigations/baths, or pastes, for example. The tobramycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the method includes combining between about 100 mg and about 1.5 g tobramycin with the carrier to formulate an administration dosage. For instance, one or more compounded capsules comprising about 100 mg tobramycin may be combined with the carrier. The compounded capsule may be opened or otherwise manipulated to release the contents for combining with the carrier. In another example, the method includes combining all or portion of a commercially available 1.2 gram vial of Tobramycin Sulfate Injection, powder, for solution with the carrier. The contents of the one or more vials or capsules may be added to a volume of the carrier sufficient to formulate an administration dosage of the topical composition having the desired strength of the tobramycin. In some embodiments, the amount of tobramycin in the administration dosage corresponds to the amount of tobramycin present in a vial, capsule, or a multiple thereof.

In various embodiments, the active agent includes tobramycin and an administration dosage of the topical composition comprises tobramycin in an amount about 20 mg or less, about 20 mg or greater, about 100 mg or greater, about 200 mg or greater, about 300 mg or greater, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.2 g or greater, about 2.5 g or greater, about 3 g or greater, about 3.5 g or greater, about 4 g or greater, between about 20 mg and about 100 mg, between about 100 mg and about 200 mg, between about 200 mg and about 400 mg, between about 400 mg and about 700 mg, between about 700 mg and about 1 g, between about 1 g and about 1.3 g, between about 1.3 g and about 1.6 g, between about 1.6 g and about 1.9 g, between about 1.9 g and about 2.4 g, between about 2.4 g and about 2.8 g, between about 2.8 g and about 3.2 g, between about 3.2 g and about 3.8 g, between about 3.8 g and about 4.4 g, between about 4.4 g and about 5 g, between about 5 g and about 6 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available tobramycin formats such as capsules, vials for injection or solution, bulk power, solutions, topical ointments, creams, lotions, and/or gels, for example. The topical composition may be administered to the skin, vagina, nasal passage, or anal cavity. The topical composition comprising tobramycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including tobramycin may be applied to skin in powder or solution followed by application of a carrier or second portion of the carrier comprising gel, lotion, ointment, or cream, which may include a second portion of tobramycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including tobramycin may be administered to a body surface to treat or prevent an opportunistic infection comprising Citrobacter freundii, Corynebacterium striatrum, Enterococcus faecalis, Escherichia coli, Streptococcus pyogenes, Streptococcus sanguinis (Viridans Group), Klebsiella pneumoniae, Morganella morganii, Proteus mirabilis, Pseudomonas aeruginosa, Staphylococcus aureus, Staphylococcus aureus, Staphylococcus epidermidis, Acinetobacter baumannii, Brucella species, Campylobacter jejuni, Enterobacter aerogenes, Enterobacter cloacae, Enterobacter sakazakii, Escherichia coli, Francisella tularensis, Haemophilus ducreyi, Haemophilus influenzae, Klebsiella (Calymmatobacterium) granulomatis, Klebsiella oxytoca, Klebsiella pneumoniae, Legionella pneumophila, Moraxella catarrhalis, Morganella morganii, Proteus mirabilis, Proteus vulgaris, Providencia sp., Pseudomonas aeruginosa, Serratia marcescens, Shigella sp., Vibrio cholerae, Yersinia pestis, Enterococcus faecalis, Staphylococcus aureus (MSSA), Staphylococcus epidermidis, Enterobacter cloacae complex, Pseudomonas aeruginosa, Providencia stuartii, and/or Proteus vulgaris. The treatment with the topical composition may also prevent or reduce an ability of such bacteria from proliferating to significantly infect the body surface or adjacent tissue.

In one embodiment, the method includes combining an active agent comprising one or more antibacterials comprising streptomycin and the carrier. The streptomycin may be combined with the carrier to formulate emulsions, lotions, creams, ointments, gels, shampoos, nail lacquers, solutions, suspensions, dispersions, irrigations/baths, or pastes, for example. The streptomycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, the streptomycin t may comprise a commercially available streptomycin, such as Streptomycin for Injection USP, which may be supplied in 1 g vials; Streptomycin Injection, Powder, Lyophilized, for Solution; or bulk powder. Other strengths may be used. The contents of the one or more vials may be added to a volume of the carrier sufficient to formulate an administration dosage of the topical composition having the desired strength of the streptomycin. In some embodiments, the amount of streptomycin in the administration dosage corresponds to the amount of streptomycin present in a vial, or a multiple thereof.

In various embodiments, the active agent includes streptomycin and an administration dosage of the topical composition comprises streptomycin in an amount about 200 mg or less, about 200 mg or greater, about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.5 g or greater, about 1.4 g or greater, between about 400 mg and about 700 mg, between about 700 mg and about 1 g, between about 1 g and about 1.3 g, between about 1.3 g and about 1.6 g, between about 1.6 g and about 2.0 g, or between about 2.0 g and about 3.0 g. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available streptomycin formats such as capsules, vials for injection or solution, bulk power, solutions, topical ointments, creams, lotions, and/or gels, for example. The topical composition may be administered to the skin, vagina, nasal passage, or anal cavity. The topical composition comprising streptomycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including streptomycin may be applied to skin in powder or solution followed by application of a carrier or second portion of the carrier comprising gel, lotion, ointment, or cream, which may include a second portion of streptomycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. The treatment with the topical composition may also prevent or reduce an ability of such bacteria from proliferating to significantly infect the body surface or adjacent tissue.

In one embodiment, the method includes combining an active agent comprising one or more antibacterials comprising sulfamethoxazole and trimethoprim and the carrier. The sulfamethoxazole and trimethoprim may be combined with the carrier to formulate emulsions, lotions, creams, ointments, gels, shampoos, nail lacquers, solutions, suspensions, dispersions, irrigations/baths, or pastes, for example. The sulfamethoxazole and trimethoprim may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

Sulfamethoxazole and trimethoprim may be combined at a weight ratio between about 1:2 to about 4:1. In one formulation, the method comprises adding about 80 mg sulfamethoxazole for about every 50 mg trimethoprim. In one embodiment, an administration volume of the topical composition comprises about 80 mg sulfamethoxazole and about 50 mg of trimethoprim. In some embodiments, the topical composition comprises between about 80 mg to about 160 mg sulfamethoxazole and between about 50 mg and about 100 mg trimethoprim, between about 160 mg to about 320 mg sulfamethoxazole and between about 100 mg and about 150 mg trimethoprim, or greater than about 160 mg sulfamethoxazole and greater than about 150 mg trimethoprim. In an example, Sulfamethoxazole and trimethoprim may comprise a commercially available sulfamethoxazole and trimethoprim, such as Sulfamethoxazole and Trimethoprim Tablets; Sulfamethoxazole and Trimethoprim Injection; Sulfamethoxazole and Trimethoprim Suspension; or bulk powder. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available sulfamethoxazole and trimethoprim formats such as capsules, vials for injection or solution, bulk power, solutions, topical ointments, creams, lotions, and/or gels, for example. The topical composition may be administered to the skin, vagina, nasal passage, or anal cavity. The topical composition comprising sulfamethoxazole and trimethoprim may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including sulfamethoxazole and trimethoprim may be applied to skin in powder or solution followed by application of a carrier or second portion of the carrier comprising gel, lotion, ointment, or cream, which may include a second portion of sulfamethoxazole and trimethoprim and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, the topical composition including sulfamethoxazole and trimethoprim may be administered to a body surface to treat or prevent an opportunistic infection comprising Acinetobacter baumannii, Acinetobacter calcoaceticus, Acinetobacter lwoffii, Bordetella pertussis, Brucella species, Enterobacter aerogenes, Enterobacter cloacae, Enterobacter sakazakii, Escherichia coli, Haemophilus influenzae, Klebsiella (Calymmatobacterium) granulomatis, Klebsiella oxytoca, Klebsiella pneumoniae, Legionella pneumophila, Moraxella catarrhalis, Morganella morganii, Proteus mirabilis, Proteus vulgaris, Providencia sp., Salmonella typhi, Serratia marcescens, Yersinia pestis, Methicillin Resistant Staph aureus (MRSA), Staphylococcus aureus (MSSA), Staphylococcus epidermidis, Streptococcus agalactiae(B), Streptococcus pneumoniae, and/or Streptococcus pyogenes(A). The treatment with the topical composition may also prevent or reduce an ability of such bacteria from proliferating to significantly infect the body surface or adjacent tissue.

In one embodiment, the method includes combining an active agent comprising one or more antibacterials comprising vancomycin and the carrier. The vancomycin may be combined with the carrier to formulate emulsions, lotions, creams, ointments, gels, shampoos, nail lacquers, solutions, suspensions, dispersions, irrigations/baths, or pastes, for example. The vancomycin may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In an example, vancomycin may comprise vancomycin powder, such as Vancomycin Hydrochloride for Injection, USP, which is a lyophilized powder for preparing intravenous (IV) infusions. The powder may be provided in vials (e.g., bottles) containing the equivalent of 500 mg, 1 g, 5 grams, 10 grams vancomycin base. Other strengths and/or formats may be used. For example, Vancomycin Hydrochloride USP powder for oral solution, equivalent to 3.75 g, 7.5 g or 15 g vancomycin, and diluent, which may be a flavored, e.g., grape-flavored, diluent for reconstitution; Vancomycin Intravenous Solution, e.g., vancomycin hydrochloride 5 mg/mL, sodium chloride 9 mg/mL. In one example, between about 25 mg and about 100 mg, such as about 50 mg vancomycin may be combined with the carrier to formulate an administration dosage of the topical composition. In one formulation, the vancomycin is provided in a compounded capsule that may be opened or otherwise manipulated to release the contents for combining with the carrier.

In various embodiments, the active agent includes vancomycin and an administration dosage of the topical composition comprises vancomycin in an amount about 25 mg or less, about 25 mg or greater, about 50 mg or greater, about 100 mg or greater, about 125 mg or greater, about 150 mg or greater, about 200 mg or greater, about 300 mg or greater, about 400 mg or greater, about 500 mg or greater, about 600 mg or greater, between about 10 mg and about 25 mg, between about 25 mg and about 50 mg, between about 50 mg and about 75 mg, between about 75 mg and about 100 mg, between about 100 mg and about 150 mg, between about 150 mg and about 250 mg, between about 250 mg and about 350 mg, or between about 350 mg and about 600 mg. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available vancomycin formats such as capsules, vials for injection or solution, bulk power, solutions, topical ointments, creams, lotions, and/or gels, for example. The topical composition may be administered to the skin, vagina, nasal passage, or anal cavity. The topical composition comprising vancomycin may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including vancomycin may be applied to skin in powder or solution followed by application of a carrier or second portion of the carrier comprising gel, lotion, ointment, or cream, which may include a second portion of vancomycin and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In some embodiments, a method of treating a bacterial infection or reducing a likelihood of contracting a bacterial infection may comprise contacting a body surface with the topical composition wherein the active agent comprises vancomycin. The body surface may be skin, hair, nails, or mucosal tissue. In some embodiments, the body surface or adjacent tissues may be infected with a bacterium selected from one or more of Bacteroides fragilis, Clostridium perfringens, Clostridium difficile, Corynebacterium jeikeium, Corynebacterium urealyticum, Enterococcus faecalis, Methicillin Resistant Staph aureus (MRSA), Peptostreptococcus, Staphylococcus aureus (MSSA), Staphylococcus epidermidis, Streptococcus agalactiae(B), Streptococcus pneumoniae, Streptococcus pyogenes(A), Viridans group streptococci, or other bacterium, such as those described herein. The treatment with the topical composition may also prevent or reduce an ability of such bacteria from proliferating to significantly infect the body surface or adjacent tissue.

In various embodiments, the method of formulating the topical composition further comprises combining the one or more antibacterials, carrier, and one or more additional actives, such as those described herein, selected from antifungals, antivirals, NSAIDs, keratolytics, antidepressants, anticonvulsants, local anesthetics, steroids, statins, acid reducers, calcium channel blockers, antianxiety drugs, mucolytics, antihistamines, or combinations thereof. In a further or another embodiment, the method may include further combining one or more actives selected from one or more nerve depressants, muscle relaxants, NMDA (N-Methyl-D-aspartate) receptor antagonists, opiate or opioid agonists, anticholinergics and/or other active agents. As introduced above, the method may include combining a first portion of an active agent with a carrier wherein the carrier comprises a commercially manufactured medicated composition comprising a second portion of the active agent, which may include one or more pharmaceutical drugs identified herein.

In one embodiment, the method includes combining an active agent comprising two or more antibacterials with the carrier. The antibacterial drugs may comprise any combination of tablets, capsules, vials for injection or solution, bulk power, solutions, topical ointments, creams, lotions, and/or gels, for example. In one example, the combination actives may be provided in one or more compounded capsules including bulk powder wherein the compounded capsule may be opened and its contents combined with the carrier. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel.

In one embodiment, the method includes combining clindamycin and mupirocin. Clindamycin and mupirocin may be combined at a weight ratio between about 1:2 to about 8:1. In one formulation, the method comprises adding about 100 mg clindamycin for about every 20 mg mupirocin. In one embodiment, an administration volume of the topical composition comprises about 100 mg clindamycin and about 20 mg mupirocin. The clindamycin and mupirocin may be provided in one or more compounded capsules including bulk powder wherein the compounded capsule may be opened and its contents combined with the carrier. As noted above, some embodiments may include other commercially available formats of clindamycin and/or mupirocin such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining doxycycline and mupirocin. Doxycycline and mupirocin may be combined at a weight ratio between about 1:2 to about 12:1. In one formulation, the method comprises adding about 200 mg doxycycline for about every 30 mg mupirocin. In one embodiment, an administration volume of the topical composition comprises about 200 mg doxycycline and about 30 mg mupirocin. The doxycycline and mupirocin may be provided in one or more compounded capsules including bulk powder wherein the compounded capsule may be opened and its contents combined with the carrier. As noted above, some embodiments may include other commercially available formats of doxycycline and/or mupirocin such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining clindamycin and gentamicin. Clindamycin and gentamycin may be combined at a weight ratio between about 1:3 to about 3:1. In one formulation, the method comprises adding about 100 mg clindamycin for about every 80 mg gentamycin. In one embodiment, an administration volume of the topical composition comprises about 100 mg clindamycin and about 80 mg gentamycin. The clindamycin and gentamycin may be provided in one or more compounded capsules including bulk powder wherein the compounded capsule may be opened and its contents combined with the carrier. As noted above, some embodiments may include other commercially available formats of clindamycin and/or gentamycin such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining doxycycline and gentamicin. Doxycycline and gentamycin may be combined at a weight ratio between about 1:3 to about 3:1. In one formulation, the method comprises adding about 100 mg doxycycline for about every 80 mg gentamycin. In one embodiment, an administration volume of the topical composition comprises about 100 mg doxycycline and about 80 mg gentamycin. The doxycycline and gentamycin may be provided in one or more compounded capsules including bulk powder wherein the compounded capsule may be opened and its contents combined with the carrier. As noted above, some embodiments may include other commercially available formats of doxycycline and/or gentamycin such as commercially available capsules, vials for injection or solution, bulk power, solutions, topical ointments, creams, lotions, and/or gels, for example. The topical composition may be administered to the skin, vagina, nasal passage, or anal cavity.

In one embodiment, the method includes combining clindamycin, gentamycin, and mupirocin. The clindamycin and gentamycin may be combined at a weight ratio between about 1:3 to about 3:1, the mupirocin and clindamycin may be combined at a weight ratio of about 1:12 to about 4:1, and the mupirocin and gentamycin may be combined at a weight ratio of about 1:10 to about 4:1. In one embodiment, the method comprises adding about 100 mg clindamycin for every about 80 mg gentamycin and about 20 mg mupirocin. In one embodiment, an administration volume of the topical composition comprises about 20 mg mupirocin, about 100 mg clindamycin, and about 80 mg gentamycin. The clindamycin, gentamycin, and mupirocin may be provided in one or more compounded capsules including bulk powder wherein the compounded capsule may be opened and its contents combined with the carrier. As noted above, some embodiments may include other commercially available formats of clindamycin, gentamycin and/or mupirocin such as commercially available capsules, vials for injection or solution, bulk power, solutions, topical ointments, creams, lotions, and/or gels, for example. The topical composition may be administered to the skin, vagina, nasal passage, or anal cavity.

In various embodiments, the method comprises combining one or more antibacterials identified herein in combination with one or more antifungals, antivirals, local anesthetics, antidepressants, steroids, NSAIDs, statins, keratolytics, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified antibacterial in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In an embodiment, the method may include combining the active agent or a portion thereof with a carrier comprising a commercially available antifungal composition comprising combining an antibacterial agent comprising Azithromycin Oral Suspension, Ciprofloxacin Cream, Ciprofloxacin Ointment, Clindamycin Cream, Clindamycin Ointment, Clindamycin Gel, Gentamycin drops, Gentamycin Spray, Gentamycin Cream, Gentamycin Ointment, Levofloxacin Injection Solution, Levofloxacin Drops, Mupirocin Ointment, Mupirocin Cream, Tobramycin Ophthalmic Ointment, Tobramycin Ophthalmic Drops, and/or Tobramycin Otic Drops.

In an aspect, the carrier comprises a commercially available Mupirocin Ointment wherein each gram of mupirocin 2.0% ointment can contain 20 mg mupirocin in a bland water miscible ointment base (polyethylene glycol ointment, NF) comprising polyethylene glycol 400 and polyethylene glycol 3350. In an aspect, mupirocin can be commercially available, for example, as a mupirocin 2.0% ointment. In an aspect, a mupirocin 2.0% ointment may be provided in a tube, such as, for example, a 22 g tube. In an aspect, mupirocin ointment may include mupirocin cream USP containing 2.15% w/w mupirocin calcium USP (equivalent to 2% mupirocin free acid) in an oil- and water-based emulsion supplied in 15-gram and 30-gram tubes.

In one embodiment, the method includes mixing linezolid oral suspension with one or more active agents, such as an antibacterial agent or an antifungal active. An example, oral suspension may include inactive ingredients such as sucrose, citric acid, sodium citrate, microcrystalline cellulose and carboxymethylcellulose sodium, aspartame, xanthan gum, mannitol, sodium benzoate, colloidal silicon dioxide, sodium chloride, or combination thereof.

In various embodiments, the method of formulating the topical composition includes combining one or more antivirals selected from acyclovir, famciclovir, valacyclovir, penciclovir, or combination thereof with a carrier. In various embodiments, the topical composition may comprise between about 0.01% and about 50% by weight antiviral. In one embodiment, the method of formulating the topical composition comprises combining the active agent comprising an antiviral identified herein and a carrier in an amount sufficient to formulate the topical composition comprising the antiviral in an amount between about 0.01% and about 20% by weight, such as between about 0.5% and about 5% or any other percent, percent range, or percent therebetween, such as between about 2% and about 10%. In various embodiments, the active agent includes one or more antivirals and an administration dosage of the topical composition comprises an antiviral in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The active agent may also include any additional active, such as any of those described herein. The antiviral may comprise tablets, capsules, vials for injection or solution, bulk power, solutions, topical ointments, creams, lotions, and/or gels, for example. Combining the antiviral may comprise adding a bulk powder, crushed tablet, or injection powder. As described above, the carrier may include an aqueous, organic, or inorganic solution, which may include a dispersion or suspension, cream, gel, ointment, lotion, emulsion, powder, or paste. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more antivirals may be applied to skin in powder or solution followed by application of a carrier or second portion of the carrier comprising gel, lotion, ointment, or cream, which may include a second portion of the antiviral and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In various embodiments, the method comprises combining with the carrier one or more antivirals identified herein in combination with one or more antifungals, antibacterials, local anesthetics, antidepressants, NSAIDs, steroids, statins, keratolytics, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified antivirals in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more antivirals comprising acyclovir and the carrier. The acyclovir may be combined with the carrier to formulate emulsions, lotions, creams, ointments, gels, shampoos, nail lacquers, solutions, suspensions, dispersions, irrigations/baths, or pastes, for example. The acyclovir may comprise commercially available tablets, capsules, vials for injection, bulk power, solutions, topical ointments, creams, lotions, and/or gels, for example. In various embodiments, the active agent includes acyclovir and an administration dosage of the topical composition comprises acyclovir in an amount about 100 mg or less, about 100 mg or greater, about 200 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 100 mg and about 200 mg, between about 200 mg and about 400 mg, between about 400 mg and about 600 mg, between about 600 mg and about 1 g, between about 1 g and about 1.2 g, between about 1.2 g and about 1.4 g, or between about 1.6 g and about 1.8 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more or the contents of one or more acyclovir oral capsules with the carrier. The capsules may comprise 200 mg acyclovir and one or more of colloidal silicon dioxide, croscarmellose sodium, magnesium stearate, and/or microcrystalline cellulose. In another example, the capsules may contain 200 mg acyclovir and one or more of lactose monohydrate, sodium lauryl sulfate, sodium starch glycolate, and/or magnesium stearate. In another example, the capsules may contain 200 mg of acyclovir and one or more of microcrystalline cellulose, povidone, sodium starch glycolate, pregelatinized starch, and/or magnesium stearate. Other strengths may be used. The one or more capsules may be preferably opened to release the contents for combining the contents with the carrier. In an embodiment, the method includes combining one or more acyclovir tablets with the carrier. For example, one or more 400 mg or 800 mg acyclovir tablets may be ground to a fine powder and added to the carrier. Acyclovir tablets may include 400 mg or 800 mg acyclovir and one or more of colloidal silicon dioxide, magnesium stearate, microcrystalline cellulose, povidone, and/or sodium starch glycolate. Other strengths may be used. The one or more tablets may be crushed or otherwise ground to a powder, typically prior to combining with the carrier for ease of mixing. The one or more tablets and/or capsules, which may be the contents thereof, may be added to a volume of the carrier sufficient to formulate an administration dosage of the topical composition having the desired strength of the acyclovir. In some embodiments, the amount of acyclovir in the administration dosage corresponds to the amount of acyclovir present in one tablet or tablet, or a multiple thereof.

In one embodiment, the method includes combining an active agent comprising one or more antivirals comprising famciclovir and the carrier. The famciclovir may be combined with the carrier to formulate emulsions, lotions, creams, ointments, gels, shampoos, nail lacquers, solutions, suspensions, dispersions, irrigations/baths, or pastes, for example. The famciclovir may comprise commercially available tablets, capsules, vials for injection, bulk power, solutions, topical ointments, creams, lotions, and/or gels, for example. In various embodiments, an administration dosage of the topical composition comprises famciclovir in an amount about 100 mg or less, about 100 mg or greater, about 125 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more famciclovir oral tablets and the carrier. The tablets may include about 125 mg, about 250 mg or about 500 mg of famciclovir and one or more of croscarmellose sodium, crospovidone, hypromellose 2910, lactose monohydrate, magnesium stearate, polyethylene glycols 6000, and/or titanium dioxide. In one formulation, one or more tablets include about 125 mg, about 250 mg or about 500 mg of famciclovir and one or more of hypromellose, poloxamer, polyethylene glycol, stearic acid, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the famciclovir. In some embodiments, the amount of famciclovir in the administration dosage corresponds to the amount of famciclovir present in one tablet, or a multiple thereof.

In one embodiment, the method includes combining an active agent comprising one or more antivirals comprising valacyclovir and the carrier. The valacyclovir may be combined with the carrier to formulate emulsions, lotions, creams, ointments, gels, shampoos, nail lacquers, solutions, suspensions, dispersions, irrigations/baths, or pastes, for example. The valaciclovir may comprise commercially available tablets, capsules, vials for injection, bulk power, solutions, topical ointments, creams, lotions, and/or gels, for example. In various embodiments, an administration dosage of the topical composition comprises valaciclovir in an amount about 200 mg or less, about 200 mg or greater, about 500 mg or greater, about 700 mg or greater, about 1 g or greater, about 1.2 g or greater, about 1.4 g or greater, about 1.6 g or greater, about 1.8 g or greater, about 2 g or greater, about 2.5 g or greater, about 1.4 g or greater, between about 400 mg and about 700 mg, between about 700 mg and about 1 g, between about 1 g and about 1.3 g, between about 1.3 g and about 1.6 g, between about 1.6 g and about 2.0 g, or between about 2.0 g and about 3.0 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more valacyclovir oral tablets and the carrier. The tablets may include valacyclovir hydrochloride equivalent to about 500 mg or 1 g valacyclovir and one or more of crospovidone, hypromellose, magnesium stearate, microcrystalline cellulose, polyethylene glycol, polysorbate 80, coloring, and/or titanium dioxide. In another formulation, one or more valacyclovir tablets may contain valacyclovir hydrochloride equivalent to about 500 mg or about 1 g valacyclovir and one or more of croscarmellose sodium, coloring, hydrogenated castor oil, hypromellose, polyethylene glycol, polysorbate 80, starch, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the valaciclovir. In some embodiments, the amount of valaciclovir in the administration dosage corresponds to the amount of valaciclovir present in one tablet, or a multiple thereof.

In one embodiment, a method of formulating the topical composition comprises combining an active agent and carrier, wherein the active agent comprises a NSAID. The NSAID may include one or more NSAIDs selected from oxicams, such as meloxicam or piroxicam; salicylic acid derivatives, such as aspirin, diflunisal, salsalate, or trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, or tolmetin; fenamates, such as meclofenamate; and/or COX-2 inhibitors, such as celecoxib, rofecoxib, or valdecoxib. In various embodiments, the topical composition may comprise between about 0.01% and about 40% by weight NSAID, such as between about 1% and about 20% by weight. In one embodiment, the method of formulating the topical composition comprises combining the active agent comprising an NSAID identified herein and a carrier in an amount sufficient to formulate the topical composition comprising the NSAID in an amount between about 0.1% and about 40% by weight, between about 0.5% and about 30% or any other percent, percent range, or percent therebetween, such as between about 2% and about 40%. In various embodiments, the active agent includes one or more NSAIDs and an administration dosage of the topical composition comprises a NSAID in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The active agent may also include any additional active, such as any of those described herein. The NSAIDs may comprise tablets, capsules, vials for injection or solution, bulk power, solutions, topical ointments, creams, lotions, and/or gels, for example. Combining the NSAID may comprise adding a bulk powder, crushed tablet, or injection powder.

The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more NSAIDs may be applied to skin in powder or solution followed by application of a carrier or second portion of the carrier comprising gel, lotion, ointment, or cream, which may include a second portion of the NSAID and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one example, the carrier includes all or a portion of the NSAID and includes a commercially available medicated NSAID composition comprising a cream, ointment, suspension, lotion, gel, or solution. For example, the carrier may comprise a commercially available medicated NSAID composition comprising a Diclofenac Sodium Solution. Diclofenac Sodium Solution may contain, for example, 1.5% (w/w), diclofenac sodium wherein each 1 mL of solution contains about 16.05 mg of diclofenac sodium. In one embodiment, the diclofenac solution comprises a diclofenac sodium solution, 1.5% (w/w), such as that which is manufactured under the trade name PENNSAID® by Nuvo Manufacturing, Varennes, Quebec, Canada or Diclofenac Sodium Topical Solution, 1.5% (w/w), manufactured by Apotex Inc. Toronto, Ontario, Canada M9L 1T9 for Apotex Corp. Weston, Fla. 33326 for treating the pain of osteoarthritis of the knee. The diclofenac solution may also contain various inactive ingredients such as dimethyl sulfoxide USP (DMSO, 45.5% w/w), ethanol, glycerin, propylene glycol and purified water. In one embodiment, the diclofenac solution comprises a diclofenac sodium solution marketed under the trade name PENNSAID® and manufactured by Nuvo Manufacturing, Varennes, Quebec, Canada, in a 2% (w/w) diclofenac solution for treating the pain of osteoarthritis of the knee. Each gram of solution may contain about 20 mg of diclofenac sodium and various inactive ingredients such as dimethyl sulfoxide USP (DMSO, 45.5% w/w), ethanol, purified water, propylene glycol, and hydroxypropyl cellulose. In other embodiments, other concentrations of diclofenac solution, such as diclofenac sodium solutions, may be used.

In various embodiments, the method comprises combining one or more NSAIDs identified herein in combination with one or more antifungals, antibacterials, antivirals, local anesthetics, antidepressants, steroids, statins, keratolytics, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified NSAIDs in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more NSAIDs comprising celecoxib and the carrier. The celecoxib may be combined with the carrier to formulate emulsions, lotions, creams, ointments, gels, shampoos, nail lacquers, solutions, suspensions, dispersions, irrigations/baths, or pastes, for example. The celecoxib may comprise commercially available tablets, capsules, vials for injection, bulk power, solutions, topical ointments, creams, lotions, and/or gels, for example. In various embodiments, an administration dosage of the topical composition comprises celecoxib in an amount about 50 mg or less, about 50 mg, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 20 mg and about 50 mg, about 50 mg and about 100 mg, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more celecoxib oral capsules or the contents of one or more celecoxib oral capsules with the carrier. The capsules may comprise about 50 mg, about 100 mg, about 200 mg, or about 400 mg of celecoxib and one or more of croscarmellose sodium, lactose monohydrate, magnesium stearate, povidone, and/or sodium lauryl sulfate. In one formulation, the capsules may comprise about 50 mg, about 100 mg, about 200 mg, or about 400 mg of celecoxib and one or more of crospovidone, hydroxypropyl cellulose, lactose monohydrate, povidone, sodium lauryl sulfate, and/or sodium stearyl fumarate. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the celecoxib. In some embodiments, the amount of celecoxib in the administration dosage corresponds to the amount of celecoxib present in one capsule, or a multiple thereof.

In one embodiment, the method includes combining an active agent comprising one or more NSAIDs comprising etodolac and the carrier. The etodolac may be combined with the carrier to formulate emulsions, lotions, creams, ointments, gels, shampoos, nail lacquers, solutions, suspensions, dispersions, irrigations/baths, or pastes, for example. The etodolac may comprise commercially available tablets, capsules, vials for injection, bulk power, solutions, topical ointments, creams, lotions, and/or gels, for example. In various embodiments, an administration dosage of the topical composition comprises etodolac in an amount about 50 mg or less, about 50 mg, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 20 mg and about 50 mg, about 50 mg and about 100 mg, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more etodolac oral capsules or the contents of one or more etodolac oral capsules with the carrier. The capsules may include about 200 mg or about 300 mg of etodolac and one or more of lactose monohydrate, povidone, sodium starch glycolate, sodium lauryl sulfate, propylene glycol, colloidal silicon dioxide, magnesium stearate, talc, and/or titanium dioxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the etodolac. In some embodiments, the amount of etodolac in the administration dosage corresponds to the amount of etodolac present in one capsule, or a multiple thereof.

In one embodiment, the method includes combining an active agent comprising one or more NSAIDs comprising indomethacin and the carrier. The indomethacin may be combined with the carrier to formulate emulsions, lotions, creams, ointments, gels, shampoos, nail lacquers, solutions, suspensions, dispersions, irrigations/baths, or pastes, for example. The indomethacin may comprise commercially available tablets, capsules, vials for injection, bulk power, solutions, topical ointments, creams, lotions, and/or gels, for example. In various embodiments, an administration dosage of the topical composition comprises indomethacin in an amount about 25 mg or less, about 25 mg or greater, about 50 mg or greater, about 75 mg or greater, about 100 mg or greater, about 150 mg or greater, about 200 mg or greater, between about 25 mg and about 50 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more indomethacin oral capsules or the contents of one or more indomethacin oral capsules with the carrier. The capsules may comprise about 25 mg or about 50 mg indomethacin and one or more of lactose monohydrate, magnesium stearate, povidone, pregelatinized starch, silicon dioxide, sodium lauryl sulfate, sodium starch glycolate, starch, and/or titanium dioxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the indomethacin. In some embodiments, the amount of indomethacin in the administration dosage corresponds to the amount of indomethacin present in one capsule, or a multiple thereof.

In one embodiment, the method includes combining an active agent comprising one or more NSAIDs comprising nabumetone and the carrier. The nabumetone may be combined with the carrier to formulate emulsions, lotions, creams, ointments, gels, shampoos, nail lacquers, solutions, suspensions, dispersions, irrigations/baths, or pastes, for example. The nabumetone may comprise commercially available tablets, capsules, vials for injection, bulk power, solutions, topical ointments, creams, lotions, and/or gels, for example. In various embodiments, an administration dosage of the topical composition comprises nabumetone in an amount about 200 mg or less, about 200 mg or greater, about 500 mg or greater, about 750 mg or greater, about 900 mg or greater, about 1 g or greater, about 1.2 g or greater, about 1.4 g or greater, about 1.6 g or greater, about 1.8 g or greater, about 2 g or greater, about 2.5 g or greater, about 1.4 g or greater, between about 400 mg and about 750 mg, between about 750 mg and about 1 g, between about 1 g and about 1.3 g, between about 1.3 g and about 1.6 g, between about 1.6 g and about 2.0 g, or between about 2.0 g and about 3.0 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more nabumetone oral tablets and the carrier. The tablets may include about 500 mg or about 750 mg of nabumetone and one or more of hypromellose, microcrystalline cellulose, sodium lauryl sulfate, sodium starch glycolate, hypromellose, colloidal silicon dioxide, croscarmellose sodium, magnesium stearate, polysorbate 80, polyethylene glycol, sodium starch glycolate, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the nabumetone. In some embodiments, the amount of nabumetone in the administration dosage corresponds to the amount of nabumetone present in one tablet, or a multiple thereof.

In various embodiments, the method of formulating the topical composition comprises combining the active agent comprising one or more statins with a carrier in an amount, together or individually, between about 0.001% and about 45% by weight, such as less than about 1%, greater than about 1%, greater than about 5%, greater than about 15%, greater than about 25%, or greater than about 35% by weight. In various embodiments, the active agent includes one or more anticonvulsants and an administration dosage of the topical composition comprises an statin in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The one or more statins may be selected from atorvastatin, fluvastatin, pravastatin, rosuvastatin, simvastatin, or combinations thereof. The one or more statins may comprise tablets, capsules, vials for injection or solution, bulk power, solutions, topical ointments, creams, lotions, and/or gels, for example.

In one embodiment, the method of formulating the topical composition comprises combining one or more statins and a carrier in an amount sufficient to formulate the topical composition comprising the active agent in an amount between about 0.01% and about 50% by weight, such as between about 5% and about 25% or any other percent, percent range, or percent therebetween. The statin may be combined in an amount between about 0.01% and about 15% by weight, such as between about 0.5% and about 8% or any other percent, percent range, or percent therebetween. Combining the statin may comprise adding a bulk powder, crushed tablet, or injection powder. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more statins may be applied to skin in powder or solution followed by application of a carrier or second portion of the carrier comprising gel, lotion, ointment, or cream, which may include a second portion of the statin and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In various embodiments, the method comprises combining with the carrier one or more statins identified herein in combination with one or more antifungals, antibacterials, antivirals, NSAIDs, keratolytics, antidepressants, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified statins in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more statins comprising atorvastatin and the carrier. The atorvastatin may be combined with the carrier to formulate emulsions, lotions, creams, ointments, gels, shampoos, nail lacquers, solutions, suspensions, dispersions, irrigations/baths, or pastes, for example. The atorvastatin may comprise commercially available tablets, capsules, vials for injection, bulk power, solutions, topical ointments, creams, lotions, and/or gels, for example. In various embodiments, an administration dosage of the topical composition comprises atorvastatin in an amount of about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, between about 10 mg and about 25 mg, between about 25 mg and about 50 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more atorvastatin oral tablets and the carrier. The tablets may contain atorvastatin calcium equivalent to about 10 mg, about 20 mg, about 40 mg, or about 80 mg atorvastatin and one or more of calcium carbonate, croscarmellose sodium, hydroxypropyl cellulose, hypromellose, lactose monohydrate, magnesium stearate, microcrystalline cellulose, polyethylene glycol, polysorbate 80, titanium dioxide, and/or talc. In one formulation, the tablets may contain atorvastatin calcium equivalent to about 10 mg, about 20 mg, or about 40 mg atorvastatin and one or more of basic butylated methacrylate copolymer, crospovidone, hydroxy propyl cellulose, lactose monohydrate, magnesium stearate, methanol, microcrystalline cellulose, sodium bicarbonate and/or sodium lauryl sulphate. The tablets may include a film coating comprising isopropyl alcohol, methylene chloride, and a coloring agent. Other strengths may be used. The coating is preferably removed, e.g., as described herein. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the atorvastatin. In some embodiments, the amount of atorvastatin in the administration dosage corresponds to the amount of atorvastatin present in one tablet, or a multiple thereof. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available atorvastatin formats such as capsules, vials for injection, bulk power, solutions, topical ointments, creams, lotions, and/or gels, for example. The topical composition may be administered to the skin, vagina, nasal passage, or anal cavity.

In one embodiment, the method includes combining an active agent comprising one or more statins comprising fluvastatin and the carrier. The fluvastatin may be combined with the carrier to formulate emulsions, lotions, creams, ointments, gels, shampoos, nail lacquers, solutions, suspensions, dispersions, irrigations/baths, or pastes, for example. The fluvastatin may comprise commercially available tablets, capsules, vials for injection, bulk power, solutions, topical ointments, creams, lotions, and/or gels, for example. In various embodiments, an administration dosage of the topical composition comprises fluvastatin in an amount of about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, between about 10 mg and about 25 mg, between about 25 mg and about 50 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more fluvastatin oral capsules or the contents of one or more fluvastatin oral capsules with the carrier. The capsules may contain about 20 mg or about 40 mg fluvastatin sodium and one or more of magnesium stearate, crospovidone, microcrystalline cellulose, pregelatinized starch, sodium lauryl sulfate, talc, and/or titanium dioxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the fluvastatin. In some embodiments, the amount of fluvastatin in the administration dosage corresponds to the amount of fluvastatin present in one capsule, or a multiple thereof.

In one embodiment, the method includes combining an active agent comprising one or more statins comprising pravastatin and the carrier. The pravastatin may be combined with the carrier to formulate emulsions, lotions, creams, ointments, gels, shampoos, nail lacquers, solutions, suspensions, dispersions, irrigations/baths, or pastes, for example. The pravastatin may comprise commercially available tablets, capsules, vials for injection, bulk power, solutions, topical ointments, creams, lotions, and/or gels, for example. In various embodiments, an administration dosage of the topical composition comprises pravastatin in an amount of about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, about 250 mg or greater, between about 10 mg and about 25 mg, between about 25 mg and about 50 mg, between about 50 mg and about 100 mg, between about 70 mg and about 150 mg, between about 100 mg and about 200 mg, or between about 200 mg and about 250 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more pravastatin oral tablets and the carrier. The tablets may contain about 10 mg, about 20 mg, about 40 mg, or about 80 mg pravastatin sodium and one or more of croscarmellose sodium, crospovidone, magnesium stearate, meglumine, microcelac 100 (lactose monohydrate and microcrystalline cellulose), microcrystalline cellulose, and/or opadry white YS-1-7040 (hypromellose 2910, polyethylene glycol 8000, talc, and titanium dioxide). In one formulation, the tablets contain about 10 mg, about 20 mg, about 40 mg, or about 80 mg pravastatin sodium and one or more of colloidal silicon dioxide, crospovidone, hydroxypropyl methylcellulose, magnesium stearate, mannitol, meglumine, microcrystalline cellulose, coloring, and/or starch. In one formulation, the tablets contain about 10 mg, about 20 mg, about 40 mg, or about 80 mg pravastatin sodium and one or more of colloidal silicon dioxide, crospovidone, hydroxypropyl methylcellulose, magnesium stearate, mannitol, meglumine, microcrystalline cellulose, coloring, and/or starch. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the pravastatin. In some embodiments, the amount of pravastatin in the administration dosage corresponds to the amount of pravastatin present in one tablet, or a multiple thereof.

In one embodiment, the method includes combining an active agent comprising one or more statins comprising rosuvastatin and the carrier. The rosuvastatin may be combined with the carrier to formulate emulsions, lotions, creams, ointments, gels, shampoos, nail lacquers, solutions, suspensions, dispersions, irrigations/baths, or pastes, for example. The rosuvastatin may comprise commercially available tablets, capsules, vials for injection, bulk power, solutions, topical ointments, creams, lotions, and/or gels, for example. In various embodiments, an administration dosage of the topical composition comprises rosuvastatin in an amount of about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, between about 10 mg and about 25 mg, between about 25 mg and about 50 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more rosuvastatin oral tablets and the carrier. The tablets may contain about 5 mg, about 10 mg, about 20 mg, or about 40 mg of rosuvastatin and one or more of coloring, hypromellose, lactose monohydrate, light magnesium oxide, magnesium stearate, microcrystalline cellulose, titanium dioxide, and/or triacetin. In another formulation, the tablets may contain about 5 mg, about 10 mg, about 20 mg, or about 40 mg of rosuvastatin and one or more of crospovidone, dibasic calcium phosphate dihydrate, hypromellose, lactose monohydrate, magnesium stearate, microcrystalline cellulose, red ferric oxide, triacetin, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the rosuvastatin. In some embodiments, the amount of rosuvastatin in the administration dosage corresponds to the amount of rosuvastatin present in one tablet, or a multiple thereof.

In one embodiment, the method includes combining an active agent comprising one or more statins comprising simvastatin and the carrier. The simvastatin may be combined with the carrier to formulate emulsions, lotions, creams, ointments, gels, shampoos, nail lacquers, solutions, suspensions, dispersions, irrigations/baths, or pastes, for example. The simvastatin may comprise commercially available tablets, capsules, vials for injection, bulk power, solutions, topical ointments, creams, lotions, and/or gels, for example. In various embodiments, an administration dosage of the topical composition comprises simvastatin in an amount of about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, about 250 mg or greater, between about 10 mg and about 25 mg, between about 25 mg and about 50 mg, between about 50 mg and about 100 mg, between about 70 mg and about 150 mg, between about 100 mg and about 200 mg, or between about 200 mg and about 250 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more simvastatin oral tablets and the carrier. The tablets may contain about 5 mg, about 10 mg, about 20 mg, about 40 mg, or about 80 mg of simvastatin and one or more of ascorbic acid, citric acid monohydrate, hydroxypropyl cellulose, hypromellose, coloring, lactose monohydrate, magnesium stearate, microcrystalline cellulose, pregelatinized corn starch, talc, titanium dioxide, and/or butylated hydroxyanisole. In one formulation, the tablets may contain about 5 mg, about 10 mg, about 20 mg, about 40 mg, or about 80 mg of simvastatin and one or more of butylated hydroxyanisole, lactose monohydrate, magnesium stearate, microcrystalline cellulose, pregelatinized starch, talc, ascorbic acid, citric acid, hypromellose, polydextrose FCC, polyethylene glycol, titanium dioxide, and/or coloring. In another formulation, the tablets may contain about 5 mg, about 10 mg, about 20 mg, about 40 mg, or about 80 mg of simvastatin and one or more of ascorbic acid, butylated hydroxy anisole, citric acid anhydrous, hydroxypropyl cellulose, hypromellose, coloring (iron oxide), isopropyl alcohol, lactose monohydrate, magnesium stearate, microcrystalline cellulose, pregelatinized starch, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the simvastatin. In some embodiments, the amount of simvastatin in the administration dosage corresponds to the amount of simvastatin present in one tablet, or a multiple thereof.

In various embodiments, the method of formulating the topical composition comprises combining the active agent comprising one or more antidepressants with a carrier in an amount, together or individually, between about 0.0001% and about 50% by weight or any percent or range therebetween, such as between about 0.01% and about 15% by weight. In various embodiments, the active agent includes one or more antidepressants and an administration dosage of the topical composition comprises an antidepressants in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The one or more antidepressants may comprise one or more tricyclic antidepressants selected from amitriptyline, amoxapine, desipramine, doxepin, imipramine, nortriptyline, protriptyline, and/or trimipramine. In one embodiment, the antidepressant comprises one or more antidepressants selected from amitriptyline, doxepin, duloxetine, milnacipran, nortriptyline, venlafaxine, levomilnacipran, desipramine, tetracycline, or combinations thereof. In one embodiment, an antidepressant may include bupropion, mirtazapine, nefazodone, trazodone, vilazodone, and/or vortioxetine. The one or more antidepressants may comprise tablets, capsules, vials for injection or solution, bulk power, solutions, topical ointments, creams, lotions, and/or gels, for example.

In one embodiment, the method of formulating the topical composition comprises combining one or more antidepressants and a carrier in an amount sufficient to formulate the topical composition comprising the active agent in an amount between about 0.01% and about 50% by weight, such as between about 5% and about 25% or any other percent, percent range, or percent therebetween. The antidepressants may be combined in an amount between about 0.01% and about 15% by weight, such as between about 0.5% and about 8% or any other percent, percent range, or percent therebetween. Combining the antidepressants may comprise adding a bulk powder, crushed tablet, or injection powder. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more antidepressants may be applied to skin in powder or solution followed by application of a carrier or second portion of the carrier comprising gel, lotion, ointment, or cream, which may include a second portion of the antidepressant and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In various embodiments, the method comprises combining with the carrier one or more antidepressants identified herein in combination with one or more antifungals, antibacterials, antivirals, NSAIDs, keratolytics, statins, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified antidepressants in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more antidepressants comprising doxepin and the carrier. The doxepin may be combined with the carrier to formulate emulsions, lotions, creams, ointments, gels, shampoos, nail lacquers, solutions, suspensions, dispersions, irrigations/baths, or pastes, for example. The doxepin may comprise commercially available tablets, capsules, vials for injection, bulk power, solutions, topical ointments, creams, lotions, and/or gels, for example. In various embodiments, an administration dosage of the topical composition comprises doxepin in an amount of about 10 mg or greater, about 25 mg or greater, about 50 mg or greater, about 100 mg or greater, about 150 mg or greater, about 200 mg or greater, about 300 mg or greater, about 400 mg or greater, about 500 mg or greater, about 600 mg or greater, between about 10 mg and about 25 mg, between about 25 mg and about 50 mg, between about 50 mg and about 200 mg, between about 200 mg and about 300 mg, or between about 300 mg and about 600 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more doxepin oral capsules or the contents of one or more doxepin oral capsules with the carrier. The capsules may contain doxepin hydrochloride equivalent to about 10 mg, about 25 mg, about 50 mg, about 75 mg, about 100 mg, or about 150 mg of doxepin and one or more of magnesium stearate, sodium lauryl sulfate, colloidal silicon dioxide, microcrystalline cellulose, and/or pregelatinized starch. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the doxepin. In some embodiments, the amount of doxepin in the administration dosage corresponds to the amount of doxepin present in one capsule, or a multiple thereof.

In one embodiment, the method includes combining an active agent comprising one or more antidepressants comprising nortriptyline and the carrier. The nortriptyline may be combined with the carrier to formulate emulsions, lotions, creams, ointments, gels, shampoos, nail lacquers, solutions, suspensions, dispersions, irrigations/baths, or pastes, for example. The nortriptyline may comprise commercially available tablets, capsules, vials for injection, bulk power, solutions, topical ointments, creams, lotions, and/or gels, for example. In various embodiments, an administration dosage of the topical composition comprises amphotericin in an amount of about 10 mg or greater, about 25 mg or greater, about 50 mg or greater, about 75 mg or greater, about 100 mg or greater, about 150 mg or greater, about 200 mg or greater, about 250 mg or greater, between about 10 mg and about 25 mg, between about 25 mg and about 50 mg, between about 50 mg and about 75 mg, between about 75 mg and about 125 mg, between about 100 mg and about 200 mg, between about 150 mg and about 250 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more nortriptyline oral capsules or the contents of one or more nortriptyline oral capsules with the carrier. The capsules may contain nortriptyline hydrochloride equivalent to about 10 mg, about 25 mg, about 50 mg, or about 75 mg nortriptyline and one or more of colloidal silicon dioxide, magnesium stearate, pregelatinized starch, and/or sodium lauryl sulfate. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the nortriptyline. In some embodiments, the amount of nortriptyline in the administration dosage corresponds to the amount of nortriptyline present in one capsule, or a multiple thereof.

In one embodiment, the method includes combining an active agent comprising one or more antidepressants comprising amitriptyline and the carrier. The amitriptyline may be combined with the carrier to formulate emulsions, lotions, creams, ointments, gels, shampoos, nail lacquers, solutions, suspensions, dispersions, irrigations/baths, or pastes, for example. The amitriptyline may comprise commercially available tablets, capsules, vials for injection, bulk power, solutions, topical ointments, creams, lotions, and/or gels, for example. In various embodiments, an administration dosage of the topical composition comprises amitriptyline in an amount of about 10 mg or greater, about 25 mg or greater, about 50 mg or greater, about 100 mg or greater, about 150 mg or greater, about 200 mg or greater, about 250 mg or greater, about 300 mg or greater, about 350 mg or greater, about 400 mg or greater, about 450 mg or greater, between about 10 mg and about 25 mg, between about 25 mg and about 50 mg, between about 50 mg and about 100 mg, between about 100 mg and about 150 mg, between about 150 mg and about 200 mg, between about 200 mg and about 250 mg, between about 250 mg and about 300 mg, between about 300 mg and about 350 mg, or between about 350 mg and about 450 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more amitriptyline oral tablets and the carrier. The tablets may contain about 10 mg, about 25 mg, about 50 mg, about 75 mg, about 100 mg, or about 150 mg amitriptyline hydrochloride and one or more of colloidal anhydrous silica, croscarmellose sodium, lactose (monohydrate), lecithin, magnesium stearate, microcrystalline cellulose, polyvinyl alcohol, iron oxide red, talc, titanium dioxide, and/or xanthan gum. In one formulation, the tablets may contain about 10 mg, about 25 mg, about 50 mg, about 75 mg, about 100 mg, or about 150 mg amitriptyline hydrochloride and one or more of colloidal silicon dioxide, hydroxypropyl cellulose, hydroxypropyl methylcellulose, lactose (monohydrate), magnesium stearate, microcrystalline cellulose, polyethylene glycol, pregelatinized starch, and/or titanium dioxide. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the amitriptyline. In some embodiments, the amount of amitriptyline in the administration dosage corresponds to the amount of amitriptyline present in one tablet, or a multiple thereof.

In various embodiments, the active agent includes one or more mucolytics and an administration dosage of the topical composition comprises a mucolytic in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The one or more mucolytics selected from acetylcysteine, bromheksin, carbocisteine, erdosteine, guaifenesin, iodinated glycerol, pharmaceutically acceptable salts thereof, or a combination thereof. The one or more mucolytics may comprise tablets, capsules, vials for injection or solution, bulk power, solutions, topical ointments, creams, lotions, and/or gels, for example.

In one embodiment, the method of formulating the topical composition comprises combining one or more mucolytics and a carrier in an amount sufficient to formulate the topical composition comprising the active agent in an amount between about 0.01% and about 50% by weight, such as between about 5% and about 25% or any other percent, percent range, or percent therebetween. The mucolytic may be combined in an amount between about 0.01% and about 15% by weight, such as between about 0.5% and about 8% or any other percent, percent range, or percent therebetween. Combining the mucolytic may comprise adding a bulk powder, crushed tablet, or injection powder. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more mucolytics may be applied to skin in powder or solution followed by application of a carrier or second portion of the carrier comprising gel, lotion, ointment, or cream, which may include a second portion of the mucolytic and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In various embodiments, the method comprises combining one or more mucolytics identified herein in combination with one or more antifungals, antibacterials, antivirals, NSAIDs, keratolytics, statins, antidepressants, anticonvulsants, local anesthetics, steroids, acid reducers, calcium channel blockers, antianxiety drugs, or antihistamines, including combinations thereof, in an amount between about 0.001% and about 50% by weight, which is to be understood to include any weight or range of weights therebetween. In an above or another embodiment, the method comprises combining one or more identified mucolytics in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more mucolytics comprising acetylcysteine and the carrier. The acetylcysteine may be combined with the carrier to formulate emulsions, lotions, creams, ointments, gels, shampoos, nail lacquers, solutions, suspensions, dispersions, irrigations/baths, or pastes, for example. The acetylcysteine may comprise commercially available tablets, capsules, vials for injection, bulk power, solutions, topical ointments, creams, lotions, and/or gels, for example. In various embodiments, an administration dosage of the topical composition comprises acetylcysteine in an amount of about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, between about 10 mg and about 25 mg, between about 25 mg and about 50 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one embodiment, the method of formulating the topical composition comprises combining an active agent comprising a local anesthetic with the carrier. The local anesthetic may be combined with the carrier to formulate emulsions, lotions, creams, ointments, gels, shampoos, nail lacquers, solutions, suspensions, dispersions, irrigations/baths, or pastes, for example. The local anesthetic may be selected from lidocaine, prilocaine, benzocaine, or combination thereof. In various embodiments, the active agent includes one or more local anesthetics and an administration dosage of the topical composition comprises a local anesthetic in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The local anesthetic may be combined in an amount, together or individually, between about 0.0001% and about 50% by weight or any percent or range therebetween, such as between about 0.01% and about 15% by weight. For example, the local anesthetic may be combined with the carrier in an amount sufficient to formulate the topical composition comprising between about 0.01% and about 12% by weight, such as between about 0.5% and about 5% or any other percent, percent range, or percent therebetween. The active agent including the local anesthetic may be present in a combined amount by weight consistent with amounts described herein with respect to the component actives. For example, the topical composition may comprise between about 0.01% and about 12% local anesthetic, e.g., lidocaine, between about 0.001% and about 5% corticosteroid, between about 0.01% and about 50% NSAID, and/or between about 0.01% and about 15% statin by weight. Combining the local anesthetic may comprise adding a bulk powder, crushed tablet, or injection powder. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more local anesthetics may be applied to skin in powder or solution followed by application of a carrier or second portion of the carrier comprising gel, lotion, ointment, or cream, which may include a second portion of the local anesthetic and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent. In one example, the topical composition comprises an above local anesthetic in an amount described herein and flucytosine, which may include contents of a flucytosine capsule, in an amount between about 100 mg and about 1.5 g, or any range therebetween, such as those described herein. The topical composition may be in a powder format and/or formulated with a carrier comprising a powder, gel, lotion, ointment, paste, cream, shampoo, nail lacquer, or a solution or suspension for a bath, irrigation, spray, or drop application to skin or nasal mucosa.

In one example, the method of formulating the topical composition may comprise combining between about 5 mg and about 50 mg, such as about 20 mg or about 30 mg of lidocaine HCl powder to the carrier per dosage volume. The lidocaine HCl may comprise bulk powder or a compounded capsule that may be opened to remove the powder for blending with the carrier. In various embodiments, an administration dosage of the topical composition comprises lidocaine in an amount of about 5 mg or greater, about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, between about 5 mg and about 15 mg, between about 15 mg and about 25 mg, between about 15 mg and about 25 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one embodiment, the carrier comprises a commercially available medicated composition comprising a portion of the active agent as described herein. In one example, the carrier includes all or a portion of the local anesthetic and includes a commercially available medicated local anesthetic composition comprising a cream, ointment, suspension, lotion, gel, or solution. For example, the carrier may comprise a commercially available medicated Lidocaine Ointment, Lidocaine Cream, Lidocaine and Prilocaine Cream, or Lidocaine Solution. In one instance, a method of making the topical composition comprises combining the active agent with a Lidocaine Solution including lidocaine in an aqueous solution. The lidocaine solution may be a commercially available lidocaine topical solution, such as lidocaine hydrochloride solution for topical administration. The carrier may comprise the lidocaine solution. The lidocaine hydrochloride solution may contain, for example, 4% lidocaine (w/v) wherein each mL includes 40 mg lidocaine HCl. For example, in one embodiment, the lidocaine topical solution may be Lidocaine Hydrochloride Topical Solution USP, 4% manufactured by IGI Labs, Inc., Buena, N.J., in 50 mL screw cap glass bottles. The lidocaine hydrochloride topical solution may contain various inactive ingredients such as methylparaben, purified water, and sodium hydroxide to adjust pH to 6.0-7.0.

In various embodiments, the method comprises combining one or more local anesthetics identified herein in combination with one or more antifungals, antibacterials, antivirals, NSAIDs, antidepressants, steroids, statins, keratolytics, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified local anesthetics in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method of formulating the topical composition comprises combining an active agent comprising a steroid with a carrier. The steroid may be combined with the carrier to formulate emulsions, lotions, creams, ointments, gels, shampoos, nail lacquers, solutions, suspensions, dispersions, irrigations/baths, or pastes, for example. In one example, the steroid comprises a corticosteroid selected from one or more of triamcinolone (e.g., diacetate, hexacetonide, and acetonide), betamethasone (e.g., dipropionate, benzoate, sodium phosphate, acetate, and valerate), dexamethasone (e.g., dipropionate and valerate), flunisolide, prednisone (e.g., acetate), prednisolone (e.g., acetate, sodium phosphate, and tebutate), methylprednisolone (e.g., acetate and sodium succinate), fluocinolone (e.g., acetonide), budesonide, diflorasone (e.g., diacetate), halcinonide, desoximetasone (desoxymethasone), diflucortolone (e.g., valerate), flucloronide (fluocortolone acetonide), fluocinonide, fluocortolone, fluprednidene (e.g., acetate), flurandrenolide (flurandrenolone), clobetasol (e.g., propionate), clobetasone (e.g., butyrate), alclometasone, flumethasone (e.g., pivalate), fluocortolone (e.g., hexanoate), amcinonide, beclomethasone (e.g., dipropionate), fluticasone (e.g., propionate), difluprednate, prednicarbate, flurandrenolide, mometasone, desonide, halobetasol propionate, triamcinolone acetonide, or combination thereof. In another example, the steroid agent comprises a corticosteroid selected from betamethasone dipropionate, betamethasone valerate, clobetasol propionate, fluticasone propionate, fluocinonide, halcinonide, halobetasol propionate, or combination thereof. In one example, the topical composition comprises an above steroid in an amount described herein and flucytosine, which may include contents of a flucytosine capsule, in an amount between about 100 mg and about 1.5 g, or any range therebetween, such as those described herein. The topical composition may be in a powder format and/or formulated with a carrier comprising a powder, gel, lotion, ointment, paste, cream, shampoo, nail lacquer, or a solution or suspension for a bath, irrigation, spray, drop, or nebulization to skin, nasal or upper respiratory tract, or lower respiratory tract, e.g., the lungs.

In one embodiment, the method of formulating the topical composition comprises combining a steroid identified herein and a carrier in an amount, together or individually, between about 0.0001% and about 50% by weight or any percent or range therebetween, such as between about 0.001% and about 1%, between about 1% and about 3%, between about 3% and about 5%, between about 5% and about 7%, between about 7% and about 10%, between about 10% and about 15%, between about 15% and 30%, between about 30% and about 40%, between about 40% and about 50%, greater than about 5%, greater than about 7%, greater than about 10%, greater than about 15%, greater than about 20%, greater than about 30%, or greater than about 40% steroid by weight. In some embodiments, the amount of steroid by weight may be about 0.001%, about 0.005%, about 0.01%, about 0.05%, about 0.1%, about 0.5%, about 0.8%, about 1%, about 1.2%, about 1.6%, about 1.8%, about 2%, about 2.5%, about 3%, about 4%, about 5%, about 7%, about 10%, about 15%, about 20%, about 25%, about 30%, about 35%, about 40%, about 45% or any other percentage between about 0.001% and about 50% by weight of the topical composition. In various embodiments, an administration dosage of the topical composition comprises steroid in an amount of about 1 mg or greater, about 5 mg or greater, about 10 mg or greater, about 15 mg or greater, about 20 mg or greater, about 25 mg or greater, about 50 mg or greater, about 75 mg or greater, about 100 mg or greater, about 150 mg or greater, about 200 mg or greater, between about 1 mg and about 5 mg, between about 5 mg and about 15 mg, between about 15 mg and about 25 mg, between about 15 mg and about 25 mg, between about 50 mg and about 100 mg, between about 70 mg and about 150 mg, or between about 100 mg and about 200 mg. The carrier may comprise any carrier described herein, which may include a commercially available carrier or base vehicle composition for compounding or may comprise a commercially available medicated topical composition including a portion of the active agent. For example, in some embodiments, the carrier comprises a commercially available medicated composition comprising a portion of the active agent as described herein. In one example, the carrier or portion thereof includes all or a portion of the steroid and includes a commercially available medicated steroid composition comprising a cream, ointment, suspension, lotion, gel, or solution. For example, the carrier may comprise a commercially available Clobetasol Propionate Cream, Foam, Gel, or Ointment, Diflorasone Diacetate Cream or Ointment, Amcinonide Cream, Lotion, or Ointment, Betamethasone Dipropionate Cream, Lotion, Gel, or Ointment, Desoximetasone Cream or Ointment, Fluocinonide Cream, Fluocinonide Cream, Ointment, or Gel, Halcinonide Cream or Ointment, Fluocinolone Acetonide Cream, Ointment, Oil, or Solution, Halcinonide Cream or Ointment, Betamethasone Valerate Cream, Lotion, or Ointment, Diflorasone Diacetate Cream or Ointment, Triamcinolone Acetonide Cream or Ointment, Halobetasol Propionate Cream, Lotion, or Ointment, Desoximetasone Cream, Gel, or Ointment, Mometasone Furoate Cream or Ointment, Fluticasone Propionate Cream, Flurandrenolide Cream, Lotion, or Ointment, or combination thereof. In another example, the corticosteroid topical composition is selected from Clobetasol Propionate Cream or Ointment, Diflorasone Diacetate Cream or Ointment, Amcinonide Cream or Ointment, Betamethasone Dipropionate Cream or Ointment, Desoximetasone Cream or Ointment, Fluocinonide Cream or Ointment, Fluocinolone Acetonide Cream, Ointment, Oil, or Solution, Halcinonide Cream or Ointment, Triamcinolone Acetonide Cream or Ointment, Halobetasol Propionate Cream or Ointment, Mometasone Furoate Cream or Ointment, Flurandrenolide Cream or Ointment, or combination thereof. In still another example, the carrier comprises Clobetasol Propionate Cream or Ointment, Fluocinonide Cream or Ointment, Fluocinolone Acetonide Cream, Ointment, Oil, or Solution, Halcinonide Cream or Ointment, Halobetasol Propionate Cream, or Desoximetasone Cream or Ointment, Triamcinolone Acetonide Cream or Ointment, Betamethasone Dipropionate Cream or Ointment, or combination thereof. In another example, the carrier comprises Clobetasol Propionate Cream, Foam, Gel, or Ointment, 0.05%, Diflorasone Diacetate Cream or Ointment, 0.05%, Amcinonide Cream, Lotion, or Ointment, 0.1%, Betamethasone Dipropionate Cream, Lotion, Gel, or Ointment 0.05%, Desoximetasone Cream or Ointment 0.25%, Fluocinonide Cream 0.1%, Fluocinonide Cream, Ointment, or Gel, 0.05%, Fluocinolone Acetonide Cream 0.01%, Fluocinolone Acetonide Cream 0.025%, Fluocinolone Acetonide Oil 0.01%, Fluocinolone Acetonide Ointment 0.01%, Fluocinolone Acetonide Ointment 0.025%, Fluocinolone Acetonide Solution 0.01%, Halcinonide Cream or Ointment, 0.1%, Betamethasone Valerate Cream, Lotion, or Ointment 0.1%, Diflorasone Diacetate Cream or Ointment, 0.05%, Triamcinolone Acetonide Cream or Ointment, 0.1%, Triamcinolone Acetonide Ointment, 0.05%, Halobetasol Propionate Cream, Lotion, or Ointment, 0.05%, Desoximetasone Cream, Gel, or Ointment 0.05%, Mometasone Furoate Cream or Ointment, 0.1%, Fluticasone Propionate Cream, 0.05%, Flurandrenolide Cream, Lotion, or Ointment, 0.05%, or combination thereof. In still a further example of the above, a corticosteroid topical composition is selected from Clobetasol Propionate Cream or Ointment, 0.05%, Diflorasone Diacetate Cream or Ointment, 0.05%, Amcinonide Cream or Ointment, 0.1%, Betamethasone Dipropionate Cream or Ointment 0.05%, Desoximetasone Cream or Ointment 0.25%, Fluocinonide Cream 0.1%, Fluocinonide Cream or Ointment, 0.05%, Fluocinolone Acetonide Cream 0.01%, Fluocinolone Acetonide Cream 0.025%, Fluocinolone Acetonide Oil 0.01%, Fluocinolone Acetonide Ointment 0.01%, Fluocinolone Acetonide Ointment 0.025%, Fluocinolone Acetonide Solution 0.01%, Halcinonide Cream or Ointment, 0.1%, Diflorasone Diacetate Cream or Ointment, 0.05%, Triamcinolone Acetonide Cream, 0.1%, Halobetasol Propionate Cream or Ointment, 0.05%, Desoximetasone Cream or Ointment 0.05%, Mometasone Furoate Cream or Ointment, 0.1%, or Flurandrenolide Cream or Ointment, 0.05%, or combination thereof. In still a further embodiment, the corticosteroid topical composition is selected from Betamethasone Dipropionate Cream or Ointment 0.05%, Clobetasol Propionate Cream or Ointment, 0.05%, Desoximetasone Cream or Ointment 0.25%, Fluocinonide Cream 0.1%, Fluocinonide Cream or Ointment, 0.05%, Fluocinolone Acetonide Cream 0.01%, Fluocinolone Acetonide Cream 0.025%, Fluocinolone Acetonide Oil 0.011%, Fluocinolone Acetonide Ointment 0.01%, Fluocinolone Acetonide Ointment 0.025%, Fluocinolone Acetonide Solution 0.01%, Triamcinolone Acetonide Cream, 0.1%, Halobetasol Propionate Cream, 0.05%, or Desoximetasone Cream or Ointment 0.05%, or combination thereof. In a further or another example, the carrier may comprise a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more steroids may be applied to skin in powder or solution followed by application of a carrier or second portion of the carrier comprising gel, lotion, ointment, or cream, which may include a second portion of the steroid and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In one example, the topical composition comprises flucytosine, which may include contents of a flucytosine capsule, in an amount between about 100 mg and about 1.5 g, or any range therebetween, such as those described herein, and a steroid in an amount described herein. The topical composition may be in a powder format and/or formulated with a carrier comprising a powder, gel, lotion, ointment, paste, cream, shampoo, nail lacquer, or a solution or suspension for a bath, irrigation, spray, drop, or nebulization to skin, nasal or upper respiratory tract, or lower respiratory tract, e.g., the lungs.

In one embodiment, the method includes combining an active agent comprising one or more steroids comprising fluticasone and the carrier. The fluticasone may be combined with the carrier to formulate emulsions, lotions, creams, ointments, gels, shampoos, nail lacquers, solutions, suspensions, dispersions, irrigations/baths, or pastes, for example. In one example, the steroid comprises fluticasone in an amount between about 0.01% and about 2% by weight, such as between about 0.05% and about 1% or any other percent, percent range, or percent therebetween. In various embodiments, an administration dosage of the topical composition comprises fluticasone in an amount of about 1 mg or greater, about 5 mg or greater, about 10 mg or greater, about 15 mg or greater, about 20 mg or greater, about 25 mg or greater, about 50 mg or greater, between about 1 mg and about 5 mg, between about 5 mg and about 15 mg, between about 15 mg and about 25 mg, between about 15 mg and about 25 mg, or between about 50 mg and about 100 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

The active agent may be or include other actives described herein. Combining the steroid may comprise adding a bulk powder, crushed tablet, or injection powder to the carrier and blending. In one example, the method of formulating the topical composition may comprise combining between about 1 mg and about 10 mg, such as about 3 mg or about 4 mg of fluticasone powder to the carrier per dosage volume. The fluticasone may comprise bulk powder or a compounded capsule that may be opened to remove the powder for blending with the carrier. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein.

In one embodiment, the active agent comprises or consists of methylprednisolone. For example, the topical composition may include methylprednisolone in an amount between about 1 mg and about 30 mg, between about 2 mg and about 5 mg, between about 5 mg and about 8 mg, between about 8 mg and about 12 mg, between about 12 mg and about 15 mg, between about 15 mg and about 20 mg, between about 20 mg and about 35 mg, between about 35 mg and about 40 mg, about 2 mg, about 5 mg, about 7 mg, about 8 mg or greater, about 12 mg or greater, about 15 mg or greater, about 20 mg or greater, about 25 mg or greater, or about 30 mg. The methylprednisolone may include a methylprednisolone solution, suspension, emulsion, capsule, ground tablet, or powder.

In one embodiment, the method includes combining an active agent comprising one or more steroids including budesonide and the carrier. The budesonide may be combined with the carrier to formulate emulsions, lotions, creams, ointments, gels, shampoos, nail lacquers, solutions, suspensions, dispersions, irrigations/baths, or pastes, for example. The budesonide may comprise commercially available tablets, capsules, vials for injection or solution, bulk power, solutions, topical ointments, creams, lotions, and/or gels, for example. The carrier may include an aqueous, organic, or inorganic solution, which may include a dispersion or suspension, cream, gel, ointment, lotion, emulsion, powder, foam, shampoo, or paste, such as any carrier described herein. The carrier may be a commercially available carrier or base vehicle composition for compounding or may comprise a commercially available medicated topical composition including a portion of the active agent.

In one example, the budesonide comprises one or more commercially available budesonide 0.5 mg-2 ml vials and/or one or more budesonide 1 mg-2 ml vials. In another or a further example, the budesonide comprises one or more or portion of a 2 mL 0.25 mg/2 mL, 0.5 mg/2 mL, and/or 1 mg/2 mL budesonide for inhalation solution. The budesonide may be added to a volume of the carrier sufficient to formulate an administration dosage of the topical composition having the desired strength of the budesonide. In some embodiments, the amount of budesonide in the administration dosage corresponds to the amount of budesonide present in a vial or unit does container, or a multiple thereof. In various embodiments, the active agent includes budesonide and an administration dosage of the topical composition comprises budesonide in an amount about 0.5 mg or greater, about 1 mg or greater, about 1.5 mg or greater, about 2 mg or greater, about 2.5 mg or greater, about 3 mg or greater, about 3.5 mg or greater, about 4 mg or greater, about 4.5 mg or greater, about 5 mg or greater, about 5.5 mg or greater, about 6 mg or greater, about 6.5 mg or greater, about 7 mg or greater, about 7.5 mg or greater, about 8 mg or greater, between about 0.5 mg and about 30 mg, between about 0.5 mg and about 2 mg, between about 2 mg and about 4 mg, between about 4 mg and about 6 mg, between about 6 mg and about 8 mg, between about 8 mg and about 10 mg, between about 10 mg and about 12 mg, between about 12 mg and about 15 mg, between about 15 mg and about 20 mg, between about 20 mg and about 25 mg, or between about 55 mg and about 30 mg. In some embodiments, budesonide is combined with the carrier to obtain between about 0.0001% and about 50% by weight budesonide or any percent or range therebetween, such as between about 0.001% and about 1%, between about 1% and about 3%, between about 3% and about 5%, between about 5% and about 7%, between about 7% and about 10%, between about 10% and about 15%, between about 15% and 30%, between about 30% and about 40%, between about 40% and about 50%, greater than about 5%, greater than about 7%, greater than about 10%, greater than about 15%, greater than about 20%, greater than about 30%, or greater than about 40% steroid by weight. In some embodiments, the amount of budesonide by weight may be about 0.001%, about 0.005%, about 0.01%, about 0.05%, about 0.1%, about 0.5%, about 0.8%, about 1%, about 1.2%, about 1.6%, about 1.8%, about 2%, about 2.5%, about 3%, about 4%, about 5%, about 7%, about 10%, about 15%, about 20%, about 25%, about 30%, about 35%, about 40%, about 45% or any other percentage between about 0.001% and about 50% by weight of the topical composition. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include other commercially available budesonide formats such as capsules, vials for injection or solution, bulk power, solutions, topical ointments, creams, lotions, and/or gels, for example. The topical composition may be administered to the skin, vagina, nasal passage, or anal cavity. The topical composition comprising budesonide may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, the composition including one or more actives including budesonide may be applied to skin in powder or solution followed by application of a carrier or second portion of the carrier comprising gel, lotion, ointment, or cream, which may include a second portion of budesonide and/or another active or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent, which may include a portion of the budesonide or another portion of the active agent.

In various embodiments, the method comprises combining budesonide in combination with one or more antifungals, antibacterials, antivirals, NSAIDs, antidepressants, local anesthetics, statins, keratolytics, acid reducers, additional steroid, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining budesonide in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In an embodiment, the method comprises combining an active agent comprising one or more anticonvulsants and/or nerve depressants and a carrier. The anticonvulsant and/or nerve depressant may comprise tablets, capsules, vials for injection or solution, bulk power, solutions, topical ointments, creams, lotions, and/or gels, for example. In various formulations, an anticonvulsant and/or nerve depressant may comprise between about 0.0001% and about 50% by weight or any percent or range therebetween, such as between about 0.01% and about 15% by weight, about 10% and about 30% by weight of the topical composition and be selected from gabapentin, topiramate, lamotrigine, or combinations thereof. In various embodiments, the active agent includes one or more anticonvulsants and an administration dosage of the topical composition comprises an anticonvulsant in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g.

In one embodiment, the method of formulating the topical composition comprises combining one or more anticonvulsant and/or nerve depressants identified herein and a carrier in an amount sufficient to formulate the topical composition comprising the active agent in an amount between about 0.01% and about 50% by weight, such as between about 5% and about 25% or any other percent, percent range, or percent therebetween. The anticonvulsant and/or nerve depressant may be combined in an amount between about 0.01% and about 10% by weight, such as between about 0.5% and about 5% or any other percent, percent range, or percent therebetween. Combining the anticonvulsant and/or nerve depressant may comprise adding a bulk powder, crushed tablet, or injection powder. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more anticonvulsants and/or nerve depressants may be applied to skin in powder or solution followed by application of a carrier or second portion of the carrier comprising gel, lotion, ointment, or cream, which may include a second portion of the anticonvulsant and/or nerve depressant and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In various embodiments, the method comprises combining one or more anticonvulsants and/or nerve depressants identified herein in combination with one or more antifungals, antibacterials, antivirals, NSAIDs, keratolytics, antidepressants, local anesthetics, steroids, statins, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified anticonvulsants and/or nerve depressants in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more anticonvulsants comprising gabapentin and the carrier. The gabapentin may be combined with the carrier to formulate emulsions, lotions, creams, ointments, gels, shampoos, nail lacquers, solutions, suspensions, dispersions, irrigations/baths, or pastes, for example. The gabapentin may comprise commercially available tablets, capsules, vials for injection, bulk power, solutions, topical ointments, creams, lotions, and/or gels, for example. In various embodiments, an administration dosage of the topical composition comprises gabapentin in an amount of about 50 mg, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 20 mg and about 50 mg, about 50 mg and about 100 mg, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one more gabapentin oral capsules or the contents of one or more gabapentin oral capsules with the carrier. The capsules may contain about 100 mg, about 300 mg, or about 400 mg of gabapentin and one or more of corn starch, talc, magnesium stearate, mannitol, sodium lauryl sulphate, and/or titanium dioxide. Other strengths may be used. The one or more capsules may be preferably opened to release the contents for combining the contents with the carrier. The one or more capsules or contents thereof may be added to a volume of the carrier sufficient to formulate an administration dosage of the topical composition having the desired strength of the gabapentin. In some embodiments, the amount of gabapentin in the administration dosage corresponds to the amount of gabapentin present in one capsule, or a multiple thereof.

In one embodiment, the method includes combining an active agent comprising one or more anticonvulsants comprising carbamazepine and the carrier. The carbamazepine may be combined with the carrier to formulate emulsions, lotions, creams, ointments, gels, shampoos, nail lacquers, solutions, suspensions, dispersions, irrigations/baths, or pastes, for example. The carbamazepine may comprise commercially available tablets, capsules, vials for injection, bulk power, solutions, topical ointments, creams, lotions, and/or gels, for example. In various embodiments, an administration dosage of the topical composition comprises carbamazepine in an amount of about 50 mg, about 100 mg or greater, about 150 mg or greater, about 200 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, between about 20 mg and about 50 mg, about 50 mg and about 100 mg, between about 100 mg and about 300 mg, between about 300 mg and about 500 mg, between about 500 mg and about 800 mg, between about 800 mg and about 1.1 g, or between about 1.1 g and about 1.5 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more carbamazepine oral tablets and the carrier. The tablets may contain about 100 mg, about 200 mg, about 300 mg, or about 400 mg carbamazepine and one or more of colloidal silicon dioxide, coloring, hypromellose, magnesium stearate, pregelatinized maize starch, corn starch, and/or sodium starch glycolate. In another formulation, the tablets contain about 100 mg, about 200 mg, about 300 mg, or about 400 mg carbamazepine and one or more of ammonio methacrylate copolymer, corn starch, croscarmellose sodium, diethyl phthalate, magnesium stearate, and/or microcrystalline cellulose. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the carbamazepine. In some embodiments, the amount of carbamazepine in the administration dosage corresponds to the amount of carbamazepine present in one tablet, or a multiple thereof.

In one embodiment, the method includes combining an active agent comprising one or more anticonvulsants comprising lamotrigine and the carrier. The lamotrigine may be combined with the carrier to formulate emulsions, lotions, creams, ointments, gels, shampoos, nail lacquers, solutions, suspensions, dispersions, irrigations/baths, or pastes, for example. The lamotrigine may comprise commercially available tablets, capsules, vials for injection, bulk power, solutions, topical ointments, creams, lotions, and/or gels, for example. In various embodiments, an administration dosage of the topical composition comprises lamotrigine in an amount of about 10 mg or greater, about 25 mg or greater, about 50 mg or greater, about 100 mg or greater, about 150 mg or greater, about 200 mg or greater, about 300 mg or greater, about 400 mg or greater, about 500 mg or greater, about 600 mg or greater, between about 10 mg and about 25 mg, between about 25 mg and about 50 mg, between about 50 mg and about 200 mg, between about 200 mg and about 300 mg, between about 300 mg and about 400 mg, between about 400 mg and about 500 mg, or between about 500 mg and about 600 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more lamotrigine oral tablets and the carrier. The tablets may contain about 25 mg, about 100 mg, about 150 mg, or about 200 mg lamotrigine and one or more of lactose monohydrate, magnesium stearate, microcrystalline cellulose, sodium starch glycolate, pregelatinized starch; sodium lauryl sulphate, colloidal silicon dioxide, lactose monohydrate, povidone, and/or coloring. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the lamotrigine. In some embodiments, the amount of lamotrigine in the administration dosage corresponds to the amount of lamotrigine present in one tablet, or a multiple thereof.

In one embodiment, the method includes combining an active agent comprising one or more anticonvulsants comprising topiramate and the carrier. The topiramate may be combined with the carrier to formulate emulsions, lotions, creams, ointments, gels, shampoos, nail lacquers, solutions, suspensions, dispersions, irrigations/baths, or pastes, for example. The topiramate may comprise commercially available tablets, capsules, vials for injection, bulk power, solutions, topical ointments, creams, lotions, and/or gels, for example. In various embodiments, an administration dosage of the topical composition comprises topiramate in an amount of about 10 mg or greater, about 25 mg or greater, about 50 mg or greater, about 100 mg or greater, about 150 mg or greater, about 200 mg or greater, about 300 mg or greater, about 400 mg or greater, about 500 mg or greater, about 600 mg or greater, between about 10 mg and about 25 mg, between about 25 mg and about 50 mg, between about 50 mg and about 200 mg, between about 200 mg and about 300 mg, between about 300 mg and about 400 mg, between about 400 mg and about 500 mg, or between about 500 mg and about 600 mg. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more topiramate oral tablets and the carrier. The tablets may contain about 25 mg, about 50 mg, about 100 mg, or about 200 mg topiramate and one or more of microcrystalline cellulose, pre-gelatinized starch (maize), sodium starch glycolate, magnesium stearate, hypromellose, PEG 400, polysorbate 80, colloidal silicon dioxide, hypromellose, lactose monohydrate, sodium starch glycolate, coloring, titanium dioxide, and/or talc. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the topiramate. In some embodiments, the amount of topiramate in the administration dosage corresponds to the amount of topiramate present in one tablet, or a multiple thereof.

In various embodiments, the method of formulating the topical composition comprises combining the active agent comprising one or more antianxiety drugs (e.g., buspirone), calcium channel blockers (e.g., amlodipine), and/or antihistamines with a carrier in an amount, together or individually between about 0.0001% and about 50% by weight or any percent or range therebetween, such as between about 0.01% and about 15% by weight. In various embodiments, the active agent includes antianxiety drugs, calcium channel blockers, and/or antihistamines and an administration dosage of the topical composition comprises an antianxiety drug, calcium channel blocker, and/or antihistamine in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The antihistamines may be selected from acrivastine, azelastine, bilastine, bromodiphenhydramine, brompheniramine, buclizine, carbinoxamine, cetirizine, chlorodiphenhydramine, chlorphenamine, chlorpheniramine, chlorpromazine, cimetidine, clemastine, cyclizine, cyproheptadine, desloratadine, dexbrompheniramine, dexchlorpheniramine, dimenhydrinate, dimetindene, diphenhydramine, doxylamine, ebastine, embramine, emedastine, famotidine, fexofenadine, hydroxyzine, lafutidine, levocabastine, loratadine, meclozine, mirtazapine, nizatidine, olopatadine, orphenadrine, phenindamine, pheniramine, phenyltoloxamine, promethazine, pyrilamine, quetiapine, ranitidine, roxatidine, rupatadine, tiotidine, tripelennamine, triprolidine, or combination thereof. The antianxiety drugs, calcium channel blockers, and/or antihistamines may comprise tablets, capsules, vials for injection or solution, bulk power, solutions, topical ointments, creams, lotions, and/or gels, for example.

In one embodiment, the method of formulating the topical composition comprises combining one or more antianxiety drugs, calcium channel blockers, and/or antihistamines and a carrier in an amount sufficient to formulate the topical composition comprising the active agent in an amount between about 0.01% and about 50% by weight, such as between about 5% and about 25% or any other percent, percent range, or percent therebetween. The antianxiety drugs (e.g., buspirone) and/or calcium channel blockers (e.g., amlodipine) may be combined in an amount between about 0.01% and about 15% by weight, such as between about 0.5% and about 8% or any other percent, percent range, or percent therebetween. The antihistamines may be combined in an amount between about 0.0001% and about 5% by weight, such as between about 0.0005% and about 0.5% or any other percent, percent range, or percent therebetween. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more antianxiety drugs, calcium channel blockers, and/or antihistamines may be applied to skin in powder or solution followed by application of a carrier or second portion of the carrier comprising gel, lotion, ointment, or cream, which may include a second portion of the antianxiety drugs, calcium channel blockers, and/or antihistamines and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In one embodiment, the method includes combining an active agent comprising one or more antianxiety drugs (e.g., buspirone), calcium channel blockers (e.g., amlodipine), and/or antihistamines (e.g., azelastine) and the carrier. The antianxiety drugs, calcium channel blockers, and/or antihistamines may be combined with the carrier to formulate emulsions, lotions, creams, ointments, gels, shampoos, nail lacquers, solutions, suspensions, dispersions, irrigations/baths, or pastes, for example. Combining the antianxiety drugs, calcium channel blockers, and/or antihistamines may comprise adding a bulk powder, crushed tablet, or injection powder. In one example, the method includes combining one or more buspirone hydrochloride oral tablets (5 mg, 10 mg, 15 mg) and the carrier. The tablets may contain buspirone hydrochloride equivalent to about 4.6 mg, about 9.1 mg, or about 13.7 mg of buspirone free base and one or more of colloidal silicon dioxide, lactose monohydrate, magnesium stearate, microcrystalline cellulose, and/or sodium starch glycolate. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the buspirone. In some embodiments, the amount of buspirone in the administration dosage corresponds to the amount of buspirone present in one tablet, or a multiple thereof. In various embodiments, an administration dosage of the topical composition comprises buspirone in an amount of about 1 mg or greater, about 5 mg or greater, about 10 mg or greater, about 15 mg or greater, about 20 mg or greater, about 25 mg or greater, about 50 mg or greater, about 75 mg or greater, between about 1 mg and about 5 mg, between about 5 mg and about 15 mg, between about 15 mg and about 25 mg, between about 15 mg and about 25 mg, between about 50 mg and about 75 mg. In another example, the method includes combining one or more amlodipine besylate oral tablets and the carrier. The tablets may contain amlodipine besylate equivalent to about 2.5 mg, about 5 mg, or about 10 mg of amlodipine and one or more of dibasic calcium phosphate (anhydrous), butylated hydroxytoluene, microcrystalline cellulose, sodium starch glycolate, magnesium stearate, and/or dibasic calcium phosphate anhydrous. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the amlodipine. In some embodiments, the amount of amlodipine in the administration dosage corresponds to the amount of amlodipine present in one tablet, or a multiple thereof. In various embodiments, an administration dosage of the topical composition comprises lidocaine in an amount of about 1 mg or greater, about 5 mg or greater, about 10 mg or greater, about 15 mg or greater, about 20 mg or greater, about 25 mg or greater, about 25 mg or greater, about 45 mg or greater, between about 1 mg and about 5 mg, between about 5 mg and about 15 mg, between about 15 mg and about 25 mg, between about 15 mg and about 25 mg, between about 25 mg and about 50 mg. In another example, the method of formulating the topical composition may comprise combining between about 1 mcg and about 30 mcg, such as about 3 mcg or greater, about 7 mcg or greater, about 13 mcg or greater, about 17 mcg or greater, about 23 mcg or greater, about 27 mcg or greater, or about 33 mcg or greater of azelastine HCl, such as azelastine HCl powder, to the carrier per dosage volume. The azelastine HCl may comprise bulk powder or a compounded capsule that may be opened to remove the powder for blending with the carrier. As described above, the carrier may include an aqueous, organic, or inorganic solution, which may include a dispersion or suspension, cream, gel, ointment, lotion, emulsion, powder, or paste. The carrier may be a commercially available base vehicle for compounding or may be formulated as indicated elsewhere herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. In one embodiment, the carrier comprises a commercially available medicated composition comprising a portion of the active agent as described herein.

In various embodiments, the method comprises combining with the carrier one or more antianxiety drugs, calcium channel blockers, and/or antihistamines identified herein in combination with one or more antifungals, antibacterials, antivirals, NSAIDs, keratolytics, antidepressants, anticonvulsants, local anesthetics, steroids, acid reducers, or mucolytics, including combinations thereof in an amount between about 0.001% and about 50% by weight, which is to be understood to include any weight or range of weights therebetween. In an above or another embodiment, the method comprises combining one or more identified antianxiety drugs, calcium channel blockers, and/or antihistamines in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method of formulating the topical composition comprises combining an active agent comprising one or more keratolytics and a carrier. The keratolytic may be selected from urea, salicylic acid, papain, or combinations thereof. For example, the method may include combining urea and the carrier. In various embodiments, the topical composition may comprise between about 1% and about 30% by weight urea.

In one embodiment, the method of formulating the topical composition comprises combining one or more keratolytics identified herein and a carrier in an amount sufficient to formulate the topical composition comprising the active agent in an amount between about 0.01% and about 50% by weight, such as between about 5% and about 25% or any other percent, percent range, or percent therebetween. The keratolytic agent may be combined in an amount between about 5% and about 40% by weight, such as between about 10% and about 40% or any other percent, percent range, or percent therebetween. Combining the keratolytic may comprise adding a bulk powder, crushed tablet, e.g., crushed urea tablet, or injection powder. As described above, the carrier may include an aqueous, organic, or inorganic solution, which may include a dispersion or suspension, cream, gel, ointment, lotion, emulsion, powder, or paste. In one embodiment, the carrier comprises a commercially available medicated composition comprising a portion of the active agent as described herein. The carrier may comprise any carrier described herein, which may include a commercially available carrier or base vehicle composition for compounding or may comprise a commercially available medicated topical composition including a portion of the active agent. In one example, the carrier includes all or a portion of the keratolytic agent and includes a commercially available medicated keratolytic comprising a urea ointment or cream. For example, the carrier may comprise REA LO 40®, which is a 40.0% urea cream. Each gram of REA LO 40® contains 400 mg urea as the active ingredient and the following inactive ingredients: purified water, emulsifying wax, glycerin, isopropyl myristate, sorbitol, neopentyl glycol dicaprylate/dicaprate, tridecyl stearate, tridecyl trimellitate and dimethyl isosorbide. The urea cream may comprise various percentages of urea by weight (prior to compounding or prior to combination with another carrier or carrier component), such as 10%, 15%, 20%, 25%, 30%, 35%, 40%, or any other commercially available percentage by weight. In various embodiments, the urea cream may be Urix 40 Urea Cream marketed by Topix Pharmaceuticals, Inc. Urix 40 Urea Cream includes 40% urea or 400 mg urea per gram and further includes Carbomer, Cyclomethicone, Dimethicone Silyate, Dimethiconol, Glycerin, Hydrogenated Lecithin, Imidazolidinyl Urea, Petrolatum, Phenyl Trimethicone, Polyphosphorylcholine Glycol Acrylate, Triethanolamine, Water, and Xanthan Gum. In additional embodiments, the urea cream may be Rea Lo 40 topical or Rea Lo 30 topical marketed by Crown Laboratories. Rea Lo 40 topical comprises 400 mg urea per gram and Rea Lo 30 topical comprises 300 mg urea per gram. Rea Lo 40 topical and Rea Lo 30 topical further include purified water, emulsifying wax, glycerin, isopropyl myristate, sorbitol, neopentyl, glycol dicaprylate/dicaprate, tridecyl stearate, tridecyl trimellitate and dimethyl isosorbide. In additional embodiments, the urea cream may be Urea 10% Cream by Stratus Pharmaceuticals, Inc. Urea 10% Cream includes 10% urea or 100 mg urea per gram, and further includes Carbomer, Fragrance, Isopropyl Myristate, Isopropyl Palmitate, Propylene Glycol, Purified Water, Sodium Laureth Sulfate, Stearic Acid, Trolamine and Xanthan Gum. It is to be understood that the above urea creams (or any other urea cream) may be diluted or cut prior to or, in some embodiments, after compounding or otherwise combining the urea cream with additional creams and/or actives. Thus, the topical composition may comprise less urea by weight than was present in the urea cream prior to compounding or combination with another cream and/or active. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more antianxiety drugs, calcium channel blockers, and/or antihistamines may be applied to skin in powder or solution followed by application of a carrier or second portion of the carrier comprising gel, lotion, ointment, or cream, which may include a second portion of the antianxiety drugs, calcium channel blockers, and/or antihistamines and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface. In a further example, the Bassa-gel includes a portion of the active agent.

In various embodiments, the method comprises combining one or more keratolytics identified herein in combination with one or more antifungals, antibacterials, antivirals, NSAIDs, antidepressants, local anesthetics, steroids, statins, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified keratolytics in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more keratolytics comprising urea and the carrier. The urea may be combined with the carrier to formulate emulsions, lotions, creams, ointments, gels, shampoos, nail lacquers, solutions, suspensions, dispersions, irrigations/baths, or pastes, for example. In various embodiments, the active agent includes urea and an administration dosage of the topical composition comprises urea in an amount of about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The urea may comprise bulk powder or a compounded capsule that may be opened to remove the powder for blending with the carrier. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one embodiment, the method of formulating the topical composition comprises combining an active agent comprising an acid reducer with the carrier. The acid reducer may be selected from allopurinol, sucralfate, or combination thereof, for example. In various embodiments, the topical composition may comprise between about 0.01% and about 50% by weight acid reducer, such as between about 0.01% and about 5%, between about 5% and about 25%, between about 20% and about 40%, between about 30% and about 45%, between about 35% and about 50%, or any other percent, percent range, or percent therebetween by weight. In various embodiments, the active agent includes an acid reducer and an administration dosage of the topical composition comprises an acid reducer in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The active agent including the acid reducer may be present in a combined amount by weight consistent with amounts described herein with respect to the component actives. The acid reducer may comprise a bulk powder, crushed tablet, capsule, powder for injection, or a medicated cream, lotion, or ointment. The carrier may comprise any carrier described herein, which may include a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more acid reducers may be applied to skin in powder or solution followed by application of a carrier or second portion of the carrier comprising gel, lotion, ointment, or cream, which may include a second portion of the acid reducer and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface.

In various embodiments, the method comprises combining with the carrier one or more acid reducers identified herein in combination with one or more antifungals, antibacterials, antivirals, NSAIDs, keratolytics, antidepressants, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified acid reducers in combination with one or more stimulants, disinfectants, nerve depressants, muscle relaxants, NMDA (N-Methyl-D-aspartate) receptor antagonists, opiate or opioid agonists, anticholinergics and/or other active agents in an amount between about 0.01% and about 25% by weight. The one or more additional actives may comprise tablets, capsules, vials for injection or solution, bulk power, solutions, topical ointments, creams, lotions, and/or gels, for example.

In one embodiment, the method includes combining an active agent comprising one or more acid reducers comprising allopurinol and the carrier. The allopurinol may be combined with the carrier to formulate emulsions, lotions, creams, ointments, gels, shampoos, nail lacquers, solutions, suspensions, dispersions, irrigations/baths, or pastes, for example. The allopurinol may comprise commercially available tablets, capsules, vials for injection, bulk power, solutions, topical ointments, creams, lotions, and/or gels, for example. In various embodiments, the active agent includes allopurinol and an administration dosage of the topical composition comprises allopurinol in an amount of about 100 mg or greater, about 150 mg or greater, about 200 mg or greater, about 250 mg or greater, about 350 mg or greater, about 450 mg or greater, about 550 mg or greater, about 650 mg or greater, about 850 or greater, about 1 g or greater, between about 100 mg and about 200 mg, between about 200 mg and about 300 mg, between about 300 mg and about 500 mg, or between about 500 mg and about 1 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more allopurinol oral tablets and the carrier. The tablets may contain about 100 mg or about 300 mg of allopurinol and one or more of croscarmellose sodium, colloidal silicon dioxide, lactose monohydrate, magnesium stearate, pregelatinized starch, povidone, coloring, microcrystalline cellulose, pregelatinized starch, sodium lauryl sulfate. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the allopurinol. In some embodiments, the amount of allopurinol in the administration dosage corresponds to the amount of allopurinol present in one tablet, or a multiple thereof.

In one embodiment, the method includes combining an active agent comprising one or more acid reducers comprising sucralfate and the carrier. The sucralfate may be combined with the carrier to formulate emulsions, lotions, creams, ointments, gels, shampoos, nail lacquers, solutions, suspensions, dispersions, irrigations/baths, or pastes, for example. The sucralfate may comprise commercially available tablets, capsules, vials for injection, bulk power, solutions, topical ointments, creams, lotions, and/or gels, for example. In various embodiments, an administration dosage of the topical composition comprises sucralfate in an amount of about 400 mg or greater, about 600 mg or greater, about 800 mg or greater, about 1 g or greater, about 1.2 g or greater, about 1.4 g or greater, about 1.6 g or greater, about 1.8 g or greater, about 2 g or greater, about 2.5 g or greater, about 1.4 g or greater, between about 400 mg and about 700 mg, between about 700 mg and about 1 g, between about 1 g and about 1.3 g, between about 1.3 g and about 1.6 g, between about 1.6 g and about 2.0 g, or between about 2.0 g and about 3.0 g. The carrier may comprise any carrier described herein, which may include a commercially available carrier or base vehicle composition for compounding or may comprise a commercially available medicated topical composition including a portion of the active agent. The carrier may include an aqueous, organic, or inorganic solution, which may include a dispersion or suspension, cream, gel, ointment, lotion, emulsion, powder, foam, shampoo, or paste, such as any carrier described herein. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. The topical composition may be administered to the skin, vagina, nasal passage, or anal cavity.

In one example, the method includes combining one or more sucralfate oral tablets and the carrier. The tablets may contain about 1 g of sucralfate and one or more of corn starch, magnesium stearate, and/or microcrystalline cellulose. Other strengths may be used. The one or more tablets may be crushed or otherwise ground to a powder, typically prior to combining with the carrier for ease of mixing. The one or more tablets may be added to a volume of the carrier sufficient to formulate an administration dosage of the topical composition having the desired strength of the sucralfate. In some embodiments, the amount of sucralfate in the administration dosage corresponds to the amount of sucralfate present in one tablet, or a multiple thereof.

In various embodiments, the method of formulating the topical composition may include combining with the carrier one or more muscle relaxants, NMDA receptor antagonists, opiates or opioid agonists, anticholinergics, and/or stimulants, such as any identified herein.

In one example, the method comprises combining with the carrier one or more muscle relaxant selected from baclofen, carisoprodol, chlorzoxazone, cyclobenzaprine, dantrolene, diazepam, metaxalone, methocarbamol, orphenadrine, quinine sulfate, tizanidine, or combination thereof. The muscle relaxant may comprise between about 0.001% and about 50% by weight of the topical composition. In various embodiments, the active agent includes a muscle relaxant and an administration dosage of the topical composition comprises a muscle relaxant in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The muscle relaxant may comprise commercially available tablets, capsules, vials for injection, bulk power, solutions, topical ointments, creams, lotions, and/or gels, for example. In various embodiments, the method comprises combining with the carrier one or more muscle relaxants identified herein in combination with one or more antifungals, antibacterials, antivirals, NSAIDs, keratolytics, statins, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified muscle relaxants in combination with one or more stimulants, disinfectants, nerve depressants, NMDA (N-Methyl-D-aspartate) receptor antagonists, opiate or opioid agonists, anticholinergics and/or other active agents in an amount between about 0.01% and about 25% by weight. The one or more actives may comprise tablets, capsules, vials for injection or solution, bulk power, solutions, topical ointments, creams, lotions, and/or gels, for example.

In an above or another example, the method comprises combining a NMDA receptor antagonist with the carrier. The NMDA receptor antagonist may be combined in an amount between about 0.001% and 30% by weight of the topical composition. In one example, the NMDA receptor antagonist comprises ketamine. In various embodiments, the active agent includes a NMDA receptor antagonist and an administration dosage of the topical composition comprises a NMDA receptor antagonist in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g.

In one example, the method of formulating the topical composition may include combining with the carrier one or more muscle relaxant selected from baclofen, carisoprodol, chlorzoxazone, cyclobenzaprine, dantrolene, diazepam, metaxalone, methocarbamol, orphenadrine, quinine sulfate, tizanidine, or combination thereof. The muscle relaxant may comprise between about 0.001% and about 5% by weight of the topical composition. The muscle relaxant may comprise commercially available tablets, capsules, vials for injection, bulk power, solutions, topical ointments, creams, lotions, and/or gels, for example. In various embodiments, the method comprises combining with the carrier one or more muscle relaxants identified herein in combination with one or more antifungals, antibacterials, antivirals, NSAIDs, keratolytics, statins, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified muscle relaxants in combination with one or more stimulants, disinfectants, nerve depressants, NMDA (N-Methyl-D-aspartate) receptor antagonists, opiate or opioid agonists, anticholinergics and/or other active agents in an amount between about 0.01% and about 25% by weight. The one or more actives may comprise tablets, capsules, vials for injection or solution, bulk power, solutions, topical ointments, creams, lotions, and/or gels, for example.

In one example, the method of formulating the topical composition may include combining with the carrier one or more. The opiates or opioid agonists selected from tramadol; one or more C2 opiate agonists selected from oxycodone, morphine, methadone, hydromorphone, and fentanyl; one or more C3 opiate agonists selected from hydrocodone, codeine, propoxyphene, butalbital, and pentazocine; or any combination thereof. The opiate or opioid agonist may comprise between about 0.001% and about 20% by weight of the topical composition. In various embodiments, the active agent includes a opiate or opioid agonist and an administration dosage of the topical composition comprises a opiate or opioid agonist in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The opiate or opioid agonist may comprise commercially available tablets, capsules, vials for injection, bulk power, solutions, topical ointments, creams, lotions, and/or gels, for example. In various embodiments, the method comprises combining with the carrier one or more opiates or opioid agonists identified herein in combination with one or more antifungals, antibacterials, antivirals, NSAIDs, keratolytics, statins, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified opiate or opioid agonist in combination with one or more stimulants, disinfectants, nerve depressants, NMDA (N-Methyl-D-aspartate) receptor antagonists, muscle relaxants, anticholinergics and/or other active agents in an amount between about 0.01% and about 25% by weight. The one or more actives may comprise tablets, capsules, vials for injection or solution, bulk power, solutions, topical ointments, creams, lotions, and/or gels, for example.

In one example, the method of formulating the topical composition may include combining with the carrier one or more anticholinergics selected from atropine, belladonna alkaloids, benzatropine, benztropine mesylate, biperiden, bupropion, chlorpheniramine, clemastine, darifenacin, dextromethorphan, dicyclomine, dimenhydrinate, diphenhydramine, doxacurium, doxepin, doxylamine, fesoterodine, flavoxate, glycopyrrolate, hexamethonium, hydroxyzine, hyoscyamine, ipratropium (e.g., ipratropium bromide), mecamylamine, orphenadrine, oxitropium, oxybutynin, procyclidine, propantheline, scopolamine, solifenacin, tiotropium, tolterodine, trihexyphenidyl, tropicamide, tubocurarine, or a combination thereof. The anticholinergics may comprise between about 0.001% and about 40% by weight of the topical composition. In various embodiments, the active agent includes an anticholinergic and an administration dosage of the topical composition comprises an anticholinergic in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The anticholinergics may comprise commercially available tablets, capsules, vials for injection, bulk power, solutions, topical ointments, creams, lotions, and/or gels, for example. In various embodiments, the method comprises combining with the carrier one or more anticholinergics identified herein in combination with one or more antifungals, antibacterials, antivirals, NSAIDs, keratolytics, statins, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified anticholinergics in combination with one or more stimulants, disinfectants, nerve depressants, NMDA (N-Methyl-D-aspartate) receptor antagonists, muscle relaxants, opiate or opioid agonists, anticholinergics and/or other active agents in an amount between about 0.01% and about 25% by weight. The one or more actives may comprise tablets, capsules, vials for injection or solution, bulk power, solutions, topical ointments, creams, lotions, and/or gels, for example.

In one example, the method of formulating the topical composition may include combining with the carrier one or more stimulants selected from amphetamine, armodafinil, atomoxetine, benzphetamine, caffeine, doxapram, dextroamphetamine, dexmethylphenidate, dextroamphetamine, diethylpropion, methylphenidate, methamphetamine, modafinil, pitolisant, phentermine, phendimetrazine, or combination thereof. The stimulant may comprise between about 0.001% and about 50% by weight of the topical composition. In various embodiments, the active agent includes a stimulant and an administration dosage of the topical composition comprises a stimulant in an amount of about 10 mg or less, about 10 mg or greater, about 25 mg or greater, about 50 mg or greater, about 75 mg or greater, about 100 mg or greater, about 150 mg or greater, about 250 mg or greater, about 300 mg or greater, about 400 mg or greater, about 500 mg or greater, about 600 mg or greater, about 700 mg or greater, about 800 mg or greater, about 900 mg or greater, about 1 g or greater, about 1.2 g or greater, about 1.4 g or greater, about 1.5 g or greater, about 1.7 or greater, about 1.8 or greater, between about 1 mg and about 10 mg, between about 10 mg and about 25 mg, between about 25 mg and about 50 mg, between about 50 mg and about 100 mg, between about 100 mg and about 300 mg, between about 300 mg and about 500 mg, between about 500 mg and about 800 mg, between about 800 mg and about 1.1 g, between about 1.1 g and about 1.5 g, between about 1.4 g and about 1.7 g, or about 1.6 and about 1.8 g. The stimulant may comprise commercially available tablets, capsules, vials for injection, bulk power, solutions, topical ointments, creams, lotions, and/or gels, for example. In various embodiments, the method comprises combining with the carrier one or more stimulant identified herein in combination with one or more antifungals, antibacterials, antivirals, NSAIDs, keratolytics, statins, anticonvulsants, local anesthetics, steroids, acid reducers, calcium channel blockers, antianxiety drugs, mucolytics, or antihistamines, including combinations thereof in an amount between about 0.001% and about 50% by weight, which is to be understood to include any weight or range of weights therebetween. In an above or another embodiment, the method comprises combining one or more identified stimulant in combination with one or more stimulants, disinfectants, muscle relaxant, nerve depressants, NMDA (N-Methyl-D-aspartate) receptor antagonists, opiate or opioid agonists, anticholinergics and/or other active agents in an amount between about 0.01% and about 25% by weight. The one or more actives may comprise tablets, capsules, vials for injection or solution, bulk power, solutions, topical ointments, creams, lotions, and/or gels, for example.

According to any of the above embodiments wherein the active agent comprises one or more muscle relaxants, NMDA receptor antagonists, opiates or opioid agonists, anticholinergics, and/or stimulants, the topical composition may be formulated to comprise an emulsion, lotion, cream, ointment, gel, solution, dispersion, suspension, shampoo, powder, or paste and the carrier may include an aqueous, organic, or inorganic solution, which may include a dispersion or suspension, cream, gel, ointment, lotion, emulsion, powder, foam, shampoo, or paste, such as any carrier described herein. In one embodiment, the carrier comprises a commercially available carrier or base vehicle composition for compounding or may comprise a commercially available medicated topical composition including a portion of the active agent. In one example, the carrier comprises a water washable, moisturizing ointment comprising polyethylene glycol, water, Spiraea ulmaria flower extract, zinc acetate, and propylene glycol. In one embodiment, the polyethylene glycol comprises PEG-8 and PEG-75 and the carrier comprises Bassa-gel. As noted above, some embodiments may include actives comprising commercially available formats such as capsules, vials for injection or solution, bulk power, solutions, topical ointments, creams, lotions, and/or gels, for example. The topical composition may be administered to the skin, vagina, nasal passage, or anal cavity. The topical composition may be administered by contacting to a body surface, e.g., skin, mucosal tissue. The composition may be administered to an infected or target area via spray, drops, wash, swab, sponge, absorbent dressing, coating (e.g., a nail lacquer), soaking, submerging, footbath, instillation or irrigation. In various embodiments, the composition may be administered in a combinational treatment in a manner described above or elsewhere herein. For example, a topical composition comprising an active agent including one or more muscle relaxants, NMDA receptor antagonists, opiates or opioid agonists, anticholinergics, and/or stimulants may be applied to skin in powder or solution followed by application of a carrier or second portion of the carrier comprising gel, lotion, ointment, or cream, which may include a second portion of muscle relaxants, NMDA receptor antagonists, opiates or opioid agonists, anticholinergics, and/or stimulants and/or another active portion or not include a second portion of the active agent. In one embodiment, Bassa-gel is applied over the powder or solution. In another example, the body surface is soaked with an aqueous solution as described herein and then covered with the topical composition comprising a gel, lotion, cream, or ointment. In one embodiment, Bassa-gel is applied over the soaked body surface.

As introduced above, tablets may be utilized to formulate the topical composition. In some embodiments, such tablets may include film coatings. For example, film coatings may comprise polymer coatings such as a shellac. In some embodiments, the method of formulating the topical composition may include removing all or a portion of the film coating. In one example, the method includes removal of all or a portion of the film coating prior to crushing the tablet. For example, the film coating may be removed by contacting the skin/coating with a solvent. The solvent may include an alcohol, sterile sodium chloride, or aqueous solvent such as water. Contacting may include spraying or pouring the solvent onto the tablet to coat the tablet with the solvent. In one method, contacting includes submerging the tablet in an excess of solvent. The contacted tablet may be shaken with the solvent or may be allowed to rest for a sufficient period of time for solvent to act. According to one method, the contacting may be brief such as less than a minute, less than about 30 seconds, less than about 20 seconds, or between about 5 seconds and about 20 seconds. In one example, tablets coated in solvent may be quickly washed to remove the film coating after the solvent has contacted the film coating for a suitable period of time. In another method, a tablet including a film coating may be crushed and thereafter mixed with water or solvent, the powder including the film coating may then go into solution. According to a version of the method, the powder and water or solvent may be shaken to accelerate the powder going into solution.

In various embodiments, the compound topical composition may be devoid or substantially devoid of ingredients such PEGs, collagen, any active or inactive agent described herein, water, oil, carbomers, biologics, biological proteins, synthetic proteins, enzymes, nucleic acids, or chelate agents.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

Various elements described herein have been described as alternatives or alternative combinations, e.g., in a lists of selectable actives, ingredients, or compositions. It is to be appreciated that embodiments may include one, more, or all of any such elements. Thus, this description includes embodiments of all such elements independently and embodiments including such elements in all combinations.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “about” are intended to include +/−10% of the number modified.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

What is claimed is:

1. A topical composition comprising:
flucytosine, corn starch, lactose, talc, PEG-8, PEG-75, water, Spiraea ulmaria flower extract, zinc acetate, and propylene glycol.

2. The topical composition of claim 1, comprising about 250 mg and about 1500 mg of the flucytosine.

3. The topical composition of claim 1, further comprising streptomycin, vancomycin, or both.

4. A method of treating a fungal or bacterial infection in a human subject, the method comprising:
administering a topical composition to infected tissue, wherein the topical composition comprises:
flucytosine, corn starch, lactose, and talc.

5. The method of claim 4, wherein the flucytosine is present in an amount between about 250 mg and about 1500 mg.

6. The method of claim 4, wherein the topical composition is administered via nebulizing, spraying, or irrigating the infected tissue.

7. The method of claim 4, wherein the infected tissue is selected from skin, lungs, or mucosa of the upper respiratory tract.

8. The method of claim 4, wherein the topical composition is administered in a format selected from a powder, solution, or ointment.

9. The method of claim 8, wherein the flucytosine is present in an amount between about 250 mg and about 1500 mg.

10. The method of claim 8, wherein the topical composition is administered in an ointment format and further comprises PEG-8 and PEG-75, water, Spiraea ulmaria flower extract, zinc acetate, and propylene glycol.

11. The method of claim 10, wherein the infected tissue is skin.

12. The method of claim 8, wherein the topical composition is administered in a solution format comprising an aqueous solution or suspension.

13. The method of claim 12, wherein the infected tissue is selected from lungs or mucosa of the upper respiratory tract.

14. The method of claim 12, wherein the infected tissue is the lungs, and wherein the topical composition is administered via nebulization.

15. The method of claim 14, wherein the infected tissue is mucosa of the upper respiratory tract, and wherein the topical composition is administered via spray, drops, or irrigation.

16. The method of claim 8, wherein the topical composition is administered in a powder format.

17. The method of claim 16, wherein the infected tissue is skin.

18. The method of claim 16, wherein the infected tissue is mucosa of the upper respiratory tract, and the topical composition is administered nasally by inhalation or irrigation.

19. The method of claim 16, wherein the topical composition is administered via small particle nebulization.

20. The method of claim 4, further comprising administering via nasal administration gentamycin, clindamycin, mupirocin, and methylprednisolone in the same or one or more additional topical compositions.
